(19) United States
(12) Patent Application Publication
(10) Pub. No.: US 2022/0275354 A1
(43) Pub. Date: Sep. 1, 2022

(54) TEMPERATURE-SENSITIVE RNA- Guided Endonuclease

(71) Applicant: Novozymes A/S, Bagsvaerd (DK)

(72) Inventors: Brian Koebmann, Bagsvaerd (DK); Michael Dolberg Rasmussen, Vallensbæk (DK); Carsten Andersen, Vaerloese (DK)

(73) Assignee: Novozymes A/S, Bagsvaerd (DK)

(21) Appl. No.: 17/611,547

(22) PCT Filed: Apr. 29, 2020
(86) PCT No.: PCT/EP2020/061973

§ 371 Date: Nov. 15, 2021

(30) Foreign Application Priority Data
May 15, 2019 (EP) 19174696.5

(51) Int. Cl. C12N 9/22; C12N 15/11; C12N 15/75; C12N 15/90

(52) U.S. Cl. CPC C12N 9/22; C12N 15/11; C12N 15/75; C12N 15/902; C12N 2310/20; C12N 2800/80; C12N 2800/101

(57) ABSTRACT

The present invention relates to temperature-sensitive variants of an RNA-guided endonuclease, polynucleotides encoding said variants, nucleic acid constructs and expression vectors comprising polynucleotides encoding said variants, host cells expressing said variant, methods of transient repression and expression of one or more DNA target sequence using said variants, and use of said variants, polynucleotides, nucleic acid constructs, expression vectors, host cells, and methods.

### REFERENCE TO SEQUENCE LISTING

This application contains a Sequence Listing in computer-readable form, which is incorporated herein by reference.

### FIELD OF THE INVENTION

The present invention relates to temperature-sensitive variants of an RNA-guided endonuclease, polynucleotides encoding said variants, nucleic acid constructs and expression vectors comprising polynucleotides encoding said variants, host cells expressing said variant, methods of expressing and repressing one or more DNA target sequence using said variants, and use of said variants, polynucleotides, nucleic acid constructs, expression vectors, host cells, and methods.

### BACKGROUND OF THE INVENTION

The so-called CRISPR genome editing system has been widely used as a tool to modify the genomes of a number of organisms. The power of the CRISPR system lies in its simplicity to target and edit down to a single base pair in a specific gene of interest. The system relies on CRISPR-associated proteins (Cas), which are RNA-guided endonucleases, as well as so-called guide-RNA (gRNA) molecules that are able to form a complex with the endonuclease and direct the nuclease activity to a particular DNA sequence. The choice of DNA target sequence is made by varying the nucleotide sequence of the gRNA to match the target DNA sequence. When complexed with the gRNA molecule, the endonuclease can recognize and bind its target DNA sequence, forming an endonuclease-gRNA-DNA complex, and create a double-stranded break using its catalytic domain(s).

For genome editing purposes, the most widely used CRISPR-associated proteins are those of Class 2, which include Cas9 (Cas type II) derived from Streptococcus pyogenes and Cpf1 (Cas type V) derived from Acidaminococcus or Lachnospiraceae. Another example of an RNA-guided endonuclease is Mad7 isolated from Eubacterium rectale. Although there are some structural similarities between Mad7 and Cpf1, Mad7 is only 31% conserved with Cpf1 from Acidominococcus sp. at the amino acid level.

In addition to its use within genome editing, the CRISPR system can also be used to control gene expression. This application, often referred to as CRISPR interference or CRISPRi, allows sequence-specific repression or activation of a gene. CRISPR interference utilizes a catalytically inactive (“dead”) endonuclease variant (e.g., Mad7d) that can be obtained by introducing amino acid mutations in the catalytic domain responsible for endonuclease activity. Upon association with gNRA, the resulting complex retains the ability to bind to the target DNA sequence, but cannot introduce any breaks in the DNA strand. As long as the catalytically inactive endonuclease is bound to the target DNA sequence, expression of the target sequence is repressed. By varying the gRNA sequence, one can control the target DNA sequence and thereby regulate the expression of virtually any gene in any organism.

### SUMMARY OF THE INVENTION

The present invention provides temperature-sensitive variants of an RNA-guided endouclease, polynucleotides encoding said variants, nucleic acid constructs and expression vectors comprising polynucleotides encoding said variants, host cells expressing said variant, methods of expressing and repressing one or more DNA target sequence using said variants, and use of said variants, polynucleotides, nucleic acid constructs, expression vectors, host cells, and methods.

The invention is based on the surprising finding that introduction of specific amino acid alterations in the RNA-guided endonuclease Mad7 results in temperature-sensitive Mad7 variants (tsMad7). When employing these temperature-sensitive variants in vivo together with a suitable gRNA and a target DNA sequence, the initial variant-gRNA complex as well as the variant-gRNA-DNA complex become temperature-sensitive. Thus, the gRNA and DNA binding properties of the temperature-sensitive variants can be controlled by shifting the temperature up and/or down, depending on what is desired.

Thus, in a first aspect, the present invention relates to a temperature-sensitive variant of an RNA-guided endonuclease, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 2, and wherein the variant comprises at least one alteration, preferably a substitution, deletion or insertion, of an amino acid important for stability of the RNA-guided endonuclease or for stability of a complex formed between the RNA-guided endonuclease, one or more guide-RNA (gRNA), and/or one or more DNA target sequence.

In a second aspect, the present invention relates to polynucleotides encoding a variant of the first aspect.

In a third aspect, the present invention relates to nucleic acid constructs comprising a polynucleotide of the second aspect.

In a fourth aspect, the present invention relates to an expression vector comprising a nucleic acid construct of the third aspect and/or a polynucleotide of the second aspect.

In a fifth aspect, the present invention relates to a host cell comprising a variant as defined in the first aspect, a polynucleotide as defined in the second aspect, a nucleic acid construct as defined in the third aspect, and/or an expression vector as defined in the fourth aspect.

In a sixth aspect, the present invention relates to a method of inducing expression of one or more DNA target sequence of interest, the method comprising the steps of:

a) providing a host cell of the fifth aspect, said host cell further comprising one or more gRNA and one or more DNA target sequence;

b) cultivating the host cell at a permissive temperature of the variant and under conditions conducive for expression of the variant, whereby a complex is formed in the host cell between the variant with the one or more gRNA and the one or more DNA target sequence, and whereby expression of the one or more DNA target sequence is repressed; and subsequently

c) increasing the temperature to a restrictive temperature of the variant and cultivating the host cell, whereby the formed complex dissociates and expression of the one or more DNA target sequence is induced.

In a seventh aspect, the present invention relates to a method of repressing one or more DNA target sequence of interest, the method comprising the steps of:

a) providing a host cell according to the fifth aspect, said host cell further comprising one or more gRNA and one or more DNA target sequence;

b) cultivating the host cell at a restrictive temperature of the variant and under conditions conducive for expression of the variant, wherein the one or more DNA target sequence is expressed; and subsequently

c) decreasing the temperature to a permissive temperature of the variant and cultivating the host cell, whereby a complex is formed in the host cell between the variant, the one or more gRNA and the one or more DNA target sequence, and whereby expression of the one or more DNA target sequence is repressed.

The present invention also relates to use of a variant according to the first aspect, a polynucleotide according to the second aspect, a nucleic acid construct according to the third aspect, an expression vector according to the fourth aspect, a host cell according to the fifth aspect, and/or a method according to the sixth and seventh aspects.

### BRIEF DESCRIPTION OF DRAWINGS

FIG. 1 shows a schematic view of the two anti-parallel amyL gene copies inserted into the B. licheniformis SJ4671 strain mentioned in Example 1. The copies are spaced by approximately 2.5 Kb originating from non-functional DNA of the B. subtilis chromosome (SEQ ID NO: 4).

FIG. 2 shows a schematic view of an additional copy of the amyL gene inserted at the xyl locus (SEQ ID NO: 4) in the SJ4671 strain in Example 1.

FIG. 3 shows a schematic view of an additional copy of the amyL gene inserted at the gnt locus (SEQ ID NO: 5) in Example 1 to form strain SJ6026.

FIG. 4 shows a schematic view of the prsA gene inserted at the mprL locus (SEQ ID NO: 6) in Example 1 to form strain MOL2173.

FIG. 5 shows a schematic view of plasmid pBKQ3825 used for chromosomal integration of mad7d and gDNA(P4199) expressed from Pq promoter in forD locus of B. licheniformis (SEQ ID NO: 7) described in Example 2.

FIG. 6 shows a schematic view of an expression cassette consisting of the mad7d gene integrated at the forD locus and expressed from the forD promoter, the gDNA(P4199) transcribed from the PamyQsc promoter and the cat gene conferring chloramphenicol resistance (SEQ ID NO: 8) in Example 2.

FIGS. 7a and 7b show illustrations of the two B. licheniformis strains PP5007 and BKQ3934, respectively.

FIG. 8 shows results from a fed-batch experiment with B. licheniformis strains containing four copies of an amylase expressed from the P4199 promoter. Strain PP5007 contains no Mad7d, whereas strain BKQ3934 contains wt Mad7d and a gDNA(P4199). The cultures were after inoculation incubated for two days at either 30° C. or 42° C. The temperature was then shifted to 42° C. and 30° C., respectively, and cultures continued growth for additional three days. Samples were taken for measurement of amylase activities.

FIG. 9 shows a schematic view of a DNA fragment inserted in Example 4 at the amyE locus where the gfp gene encoding the green fluorescent protein is expressed from the amyL variant promoter P4199. Furthermore, a gDNA(gfp) is expressed from the PamyQ consensus promoter (PamyQsc) The final map of the amyE locus after integration is shown.

FIG. 10 shows a schematic view of an expression cassette inserted in Example 5 at the pel locus with the mad7d gene encoding the Mad7d protein.

FIG. 11 shows a schematic view of the strain MOL3268 constructed in Example 5.

FIG. 12 shows an example of the testing of different variant clones for GFP fluorescence in Example 8 at temperatures of 30° C., 34° C., 37° C., and 42° C. JA1343 (A)=negative control, strain without GFP; PP5625 (B)=GFP strain without Mad7d inhibition; MOL3268 (C)=GFP strain with wt Mad7d inhibition; TS1 to TS11=GFP strain with Mad7d-variants inhibition, strain placed at the same position in column 1 just at different temperatures as indicated 30° C., 34° C. 37° C., and 42° C. (same setup in column 2).

FIG. 13 shows GFP fluorescence in B. subtilis strains containing gfp and variants of Mad7d, cultivated in liquid TY medium at different temperatures as described in Example 9.

FIG. 14a shows a schematic view of the CRISPRi complex (MAD7d+gRNA(GFP)) binding to the GFP gene. This inhibitor complex is used to screen for TS variants in Examples 5, 8, 9 and 10.

FIG. 14b s shows a schematic view of the CRISPRi complex (MAD7d+gRNA(P4199)) binding to the promoter region of promoter P4199 to repress the expression of the gene operably linked with the P4199 promoter.

FIG. 15 shows a fed-batch experiment with B. licheniformis strains containing temperature sensitive variants of Mad7d, gDNA(P4199), and four copies of an amylase expressed from the P4199 promoter. The cultures were after inoculation incubated for two days at either 30° C. or 42° C. The temperature was then shifted to 42° C. and 30° C., respectively, and cultures continued growth for additional three days. Samples were taken for measurement of amylase activities.

FIG. 16 shows a fed-batch experiment with B. licheniformis strains containing the temperature sensitive variant TS6 of Mad7d, gDNA(P4199), and four copies of an amylase expressed from the P4199 promoter. The cultures were after inoculation incubated for two days at 42° C. The temperature was then shifted from 42° C. to 30° C., 34° C. and 39° C. or kept at 42° C. The cultures continued growth for additional three days. Samples were taken for measurement of amylase activities.

FIG. 17 shows measurements of amylase activities after incubation in liquid TY medium at 30° C., 34° C., 37° C., and 42° C. of B. licheniformis strains containing 4 C amyL expressed from P4199 promoters and temperature sensitive variants of Mad7d and gDNA(P4199).

FIG. 18 shows an alignment of the relevant region of three different Cpf1 proteins involved in endonucleolytic cleavage of DNA. The protein sequences are from the organisms Lachnospiraceae bacterium (LbCpf1), Francisella tularensis (FnCpf1) and Eubacterium rectale (Mad7). It was described in Zetsche et al., 2015, Cell 163, 759-771 that the RuvC-like domaine of FnCpf1 from F. tularensis has a conserved region where one amino acid change in position 917 from Asp (D) to Ala (A) completely inactivates the nuclease activity. The corresponding amino acid was changed in Mad7 (D877A) and was demonstrated to be inactive also.

### SEQUENCE LISTING

SEQ ID NO: 1: Polynucleotide sequence encoding Mad7 from E. rectale, codon-optimized for B. licheniformis.

SEQ ID NO: 2: Amino acid sequence of Mad7 from E. rectale.

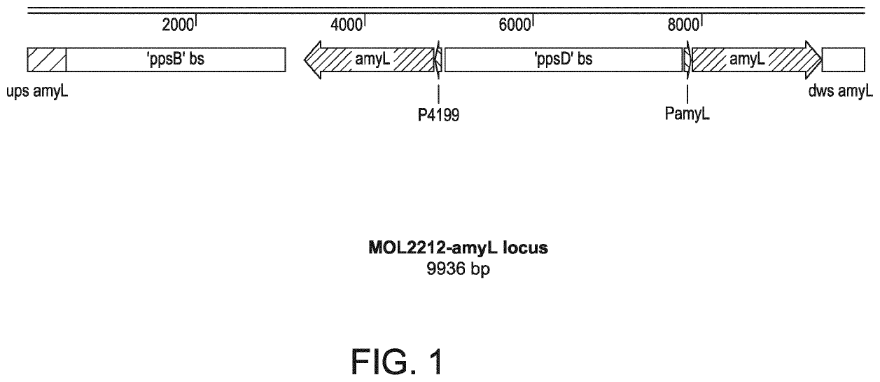

SEQ ID NO: 3: MOL2212-amyL locus, FIG. 1.

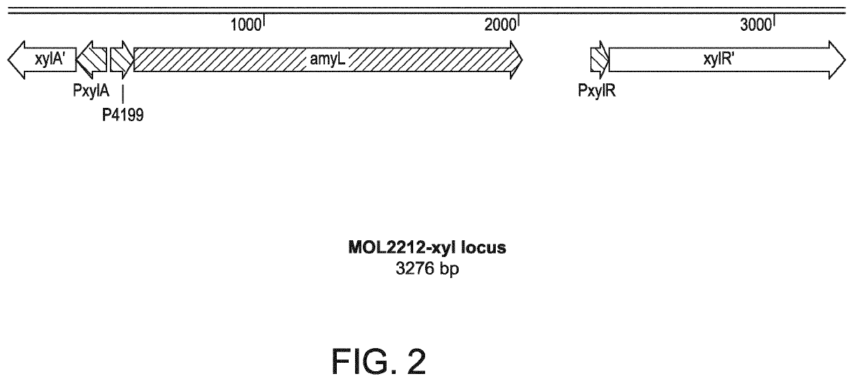

SEQ ID NO: 4: MOL2212-xyl locus, FIG. 2.

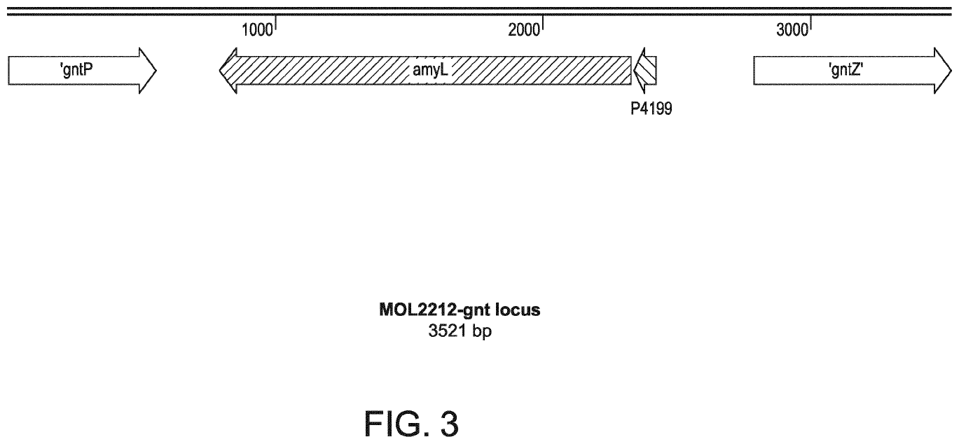

SEQ ID NO: 5: MOL2212-gntP locus, FIG. 3.

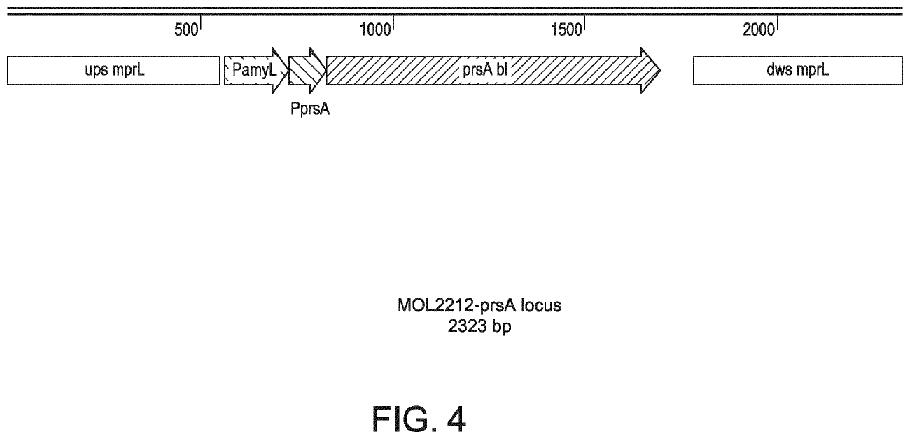

SEQ ID NO: 6: MOL2212-prsA locus, FIG. 4.

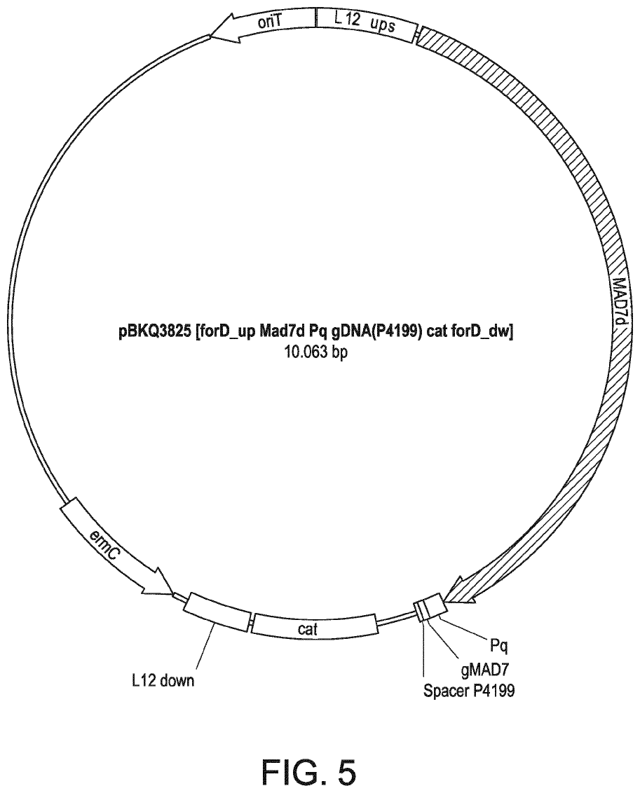

SEQ ID NO: 7: Plasmid pBKQ3825, FIG. 5 (plasmid with insertion of mad7d in between forD flanks).

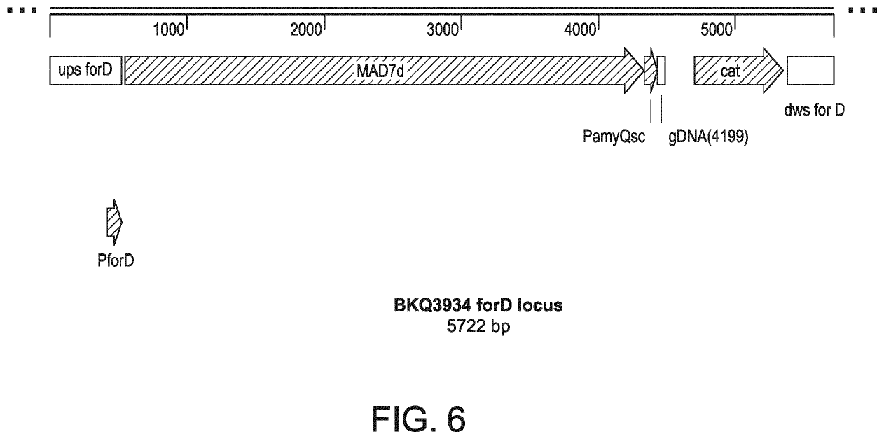

SEQ ID NO: 8: forD:mad7d-cat, FIG. 6 (BKQ3934).

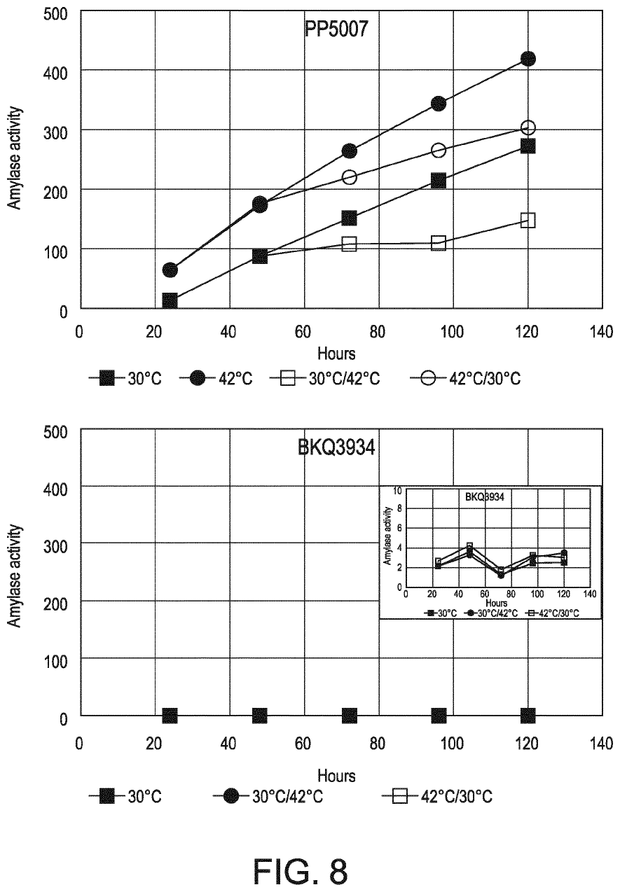

SEQ ID NO: 9: amyE:GFP-gDNA(gfp)-spc, FIG. 9 (PP5625).

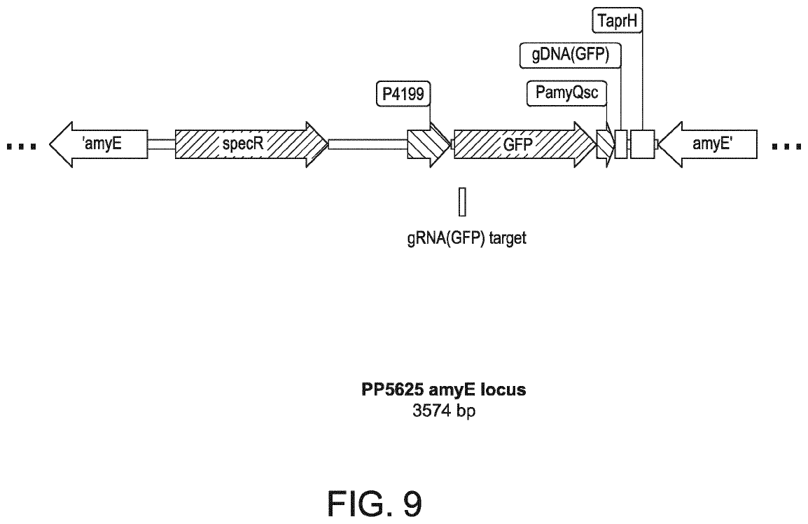

SEQ ID NO: 10: pel:mad7d, FIG. 10 (MOL3268).

SEQ ID NO: 11: gDNA(gfp).

SEQ ID NO: 12: gDNA(P4199).

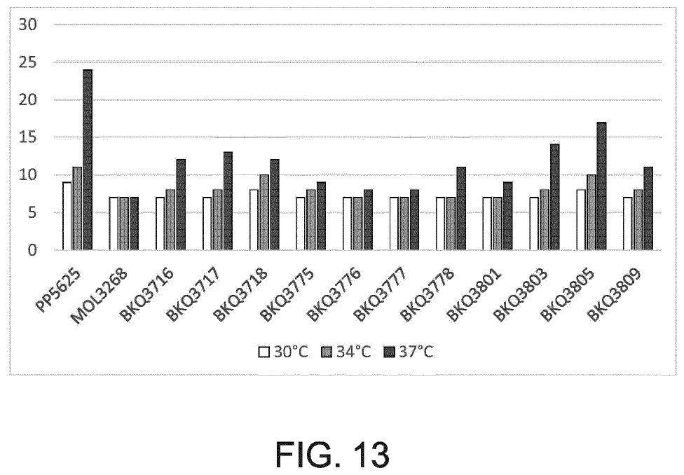

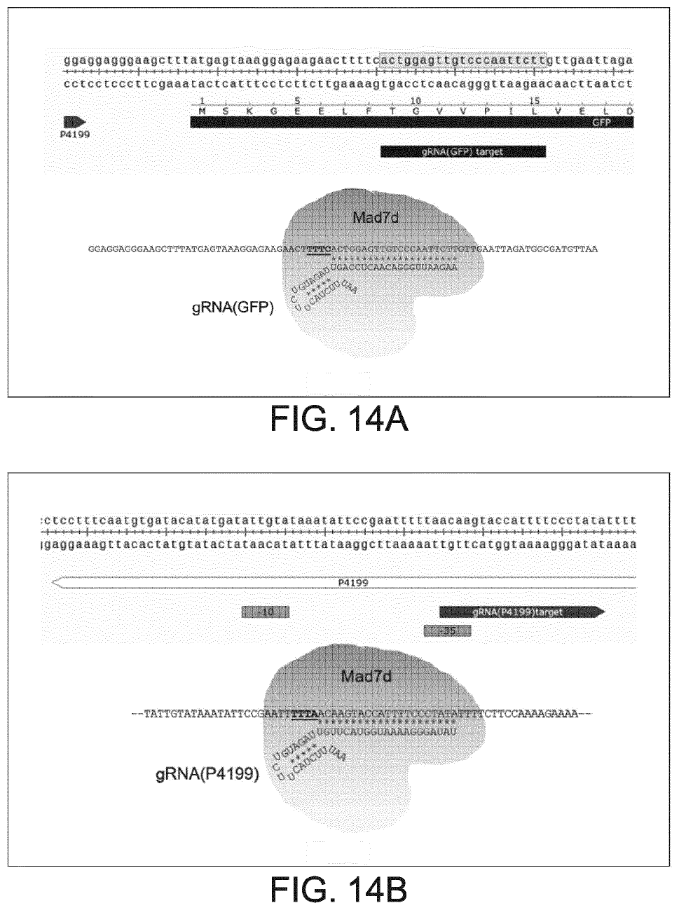

SEQ ID NO: 13: Excerpt of target DNA sequence (GFP) on FIG. 14A.

SEQ ID NO: 14: gRNA(GFP) on FIG. 14A.

SEQ ID NO: 15: Excerpt of target DNA sequence (P4199) on FIG. 14B.

SEQ ID NO: 16: gRNA(P4199) on FIG. 14B.

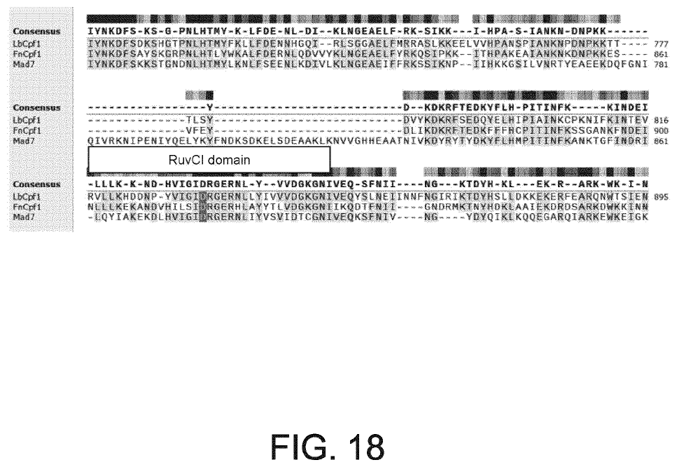

SEQ ID NO: 17: Excerpt of LbCpf1 amino acid sequence on FIG. 18.

SEQ ID NO: 18: Excerpt of FnCpf1 amino acid sequence on FIG. 18.

SEQ ID NO: 19: Excerpt of Mad7 amino acid sequence shown on FIG. 18.

### Definitions

Catalytically inactive: The term “catalytically inactive” is used to described RNA-guided endonucleases and variants thereof for which endonuclease activity has been disrupted. A catalytically inactive endonuclease can bind to its target DNA sequence, but cannot introduce any breakes in the target DNA sequence. The terms “catalytically inactive”, “nuclease-null” and “dead” (abbreviated “d”, e.g., Mad7d) are used interchangeably herein.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a variant of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Permissive temperature: The term “permissive temperature” means a temperature or temperature range, where the temperature-sensitive variant behaves as its wildtype parent. The permissive temperature is the temperature or temperature range where the variant is able to form a complex with one or more gRNA and the corresponding one or more DNA target sequence and either cut the target sequence in the case of a catalytically active variant, nick the target sequence in the case of nickase variant, or stay bound to the target sequence in the case of a catalytically inactive variant. The permissive temperature or temperature range is defined mainly by the choice of host cell and the specific temperature-sensitive variant as well as gRNA(s) applied.

Restrictive temperature: The term “restrictive temperature” means a temperature or temperature range, where the temperature-sensitive variant of the invention is unable to form a complex with one or more gRNA and the corresponding one or more DNA target sequence of interest.

RNA-guided endonuclease: The term “RNA-guided endonuclease” means a polypeptide having endonuclease activity, wherein the endonuclease activity is controlled by one or more gRNA that form a complex with the RNA-guided endonuclease and directs the endonuclease activity to a target DNA sequence that is complementary to and capable of hybridizing to the one or more gRNA.

Sequence identity: The relatedness between two amino acid sequences or between two nucleotide sequences is described by the parameter “sequence identity” or “sequence complementarity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity (or corresponding sequence complementarity) between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

To determine the % complementarity of two complementary sequences, one of the two sequences needs to be converted to its complementary sequence before the % complementarity can then be calculated as the % identity between the first sequence and the second converted sequences using the above-mentioned algorithm.

(Protein) Stability: The terms “stability” and “protein stability” are used interchangeably herein to describe the stability of the temperature-sensitive variants of the invention as well as the stability of a complex formed between the variants of the invention, one or more gRNA, and/or one or more DNA target sequence. The stability of a protein, including variants of the invention, may be defined as the net balance of forces, which determine whether a protein will be in its native folded conformation (i.e., stable, and often active) or in a denatured, unfolded, or extended state (i.e., unstable, and often inactive). In most cases, a variant of the invention will be stable at or below its permissive temperature and unstable at or above its restrictive temperature. This further applies to complexes formed between a variant of the invention, one or more gRNA, and/or one or more DNA target sequence.

Variant: The present invention relates to temperature-sensitive variants of the polypeptide of SEQ ID NO: 2 comprising at least one amino acid alteration, i.e., at least one substitution, deletion, and/or insertion at one or more (e.g., several) positions. In an embodiment, the number of alterations introduced into the polypeptide of SEQ ID NO: 2 is up to 10, e.g., 1, 2, 3, 4, 5, 6, 7, 8, 9, or 10. Alterations include conservative substitutions, wherein an amino acid is substituted with another amino acid with similar physicochemical properties; non-conservative substitutions, wherein an amino acid is substituted with another amino acid with different physicochemical properties; small insertions, typically of typically of 1-30 amino acids; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Common substitutions include, but are not limited to, Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In the context of temperature-sensitive protein variants, a conservative substitution may also be defined a substitution that has no detrimental effect on protein stability at the permissive temperature of the variant.

### Conventions for Designation of Variants

For purposes of the present invention, the polypeptide disclosed in SEQ ID NO: 2 is used to determine the corresponding amino acid residue in another RNA-guided endonuclease. The amino acid sequence of another RNA-guided endonuclease is aligned with the polypeptide disclosed in SEQ ID NO: 2, and based on the alignment, the amino acid position number corresponding to any amino acid residue in the polypeptide disclosed in SEQ ID NO: 2 may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another RNA-guided endonuclease can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other RNA-guided endonuclease has diverged from the polypeptide of SEQ ID NO: 2 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions: For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions: For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions: For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

Multiple alterations: Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations: Where different alterations can be introduced at a position, the different alterations are separated by a comma, e.g., “Arg170Tyr,Glu” and R170Y,E represents a substitution of arginine at position 170 with tyrosine or glutamic acid. Thus, “Tyr167Gly,Ala+Arg170Gly,Ala” designates the following variants:

### “Tyr167Gly+Arg170Gly”,

### “Tyr167Gly+Arg170Ala”,

### “Tyr167Ala+Arg170Gly”, and

### “Tyr167Ala+Arg170Ala”.

### DETAILED DESCRIPTION OF THE INVENTION

The present invention provides temperature-sensitive variants of an RNA-guided endouclease, polynucleotides encoding said variants, nucleic acid constructs and expression vectors comprising polynucleotides encoding said variants, host cells expressing said variant, methods of expressing and repressing one or more DNA target sequence using said variants, and use of said variants, polynucleotides, nucleic acid constructs, expression vectors, host cells, and methods.

The invention is based on the surprising finding that introduction of specific amino acid alterations in the RNA-guided endonuclease Mad7 results in temperature-sensitive Mad7 variants (tsMad7). When employing these temperature-sensitive variants in vivo together with a suitable gRNA and a target DNA sequence, the initial variant-gRNA complex as well as the variant-gRNA-DNA complex become temperature-sensitive. Thus, the gRNA and DNA binding properties of the temperature-sensitive variants can be controlled by shifting the temperature up and/or down, depending on what is desired.

### Temperature-Sensitive Variants

In a first aspect, the present invention relates to a temperature-sensitive variant of an RNA-guided endonuclease, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 2, and wherein the variant comprises at least one alteration, preferably a substitution, deletion or insertion, of an amino acid important for stability of the RNA-guided endonuclease or for stability of a complex formed between the RNA-guided endonuclease, one or more guide-RNA (gRNA), and/or one or more DNA target sequence.

The temperature-sensitive variants of the invention may be a nickase or nuclease-null variant. Preferably, a nuclease-null variant of the invention comprises an additional amino acid alteration in a position corresponding to position 877 of SEQ ID NO: 2. More preferably, such variants comprise a substitution of aspartic acid for alanine, D877A.

Temperature-sensitive variants of an RNA-guided endonuclease may be obtained from any parent RNA-guided endonuclease. Preferably, the parent RNA-guided endonuclease is a Class 2 CRISPR-associated protein. More preferably, the parent RNA-guided endonuclease is a Class 2 CRISPR-associated protein of type II or type V.

The parent RNA-guided endonuclease may be obtained from any microorganism. Preferably, the parent RNA-guided endonuclease is obtained from a prokaryote, such as bacteria or archaea. Most preferably, the parent RNA-guided endonuclease is from a Eubacterium species such as E. rectale.

In a preferred embodiment, the parent RNA-guided endonuclease has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, or 100%, to the polynucleotide sequence of SEQ ID NO: 1. Most preferably, the parent RNA-guided endonuclease comprises or consists of SEQ ID NO: 1.

A DNA sequence encoding the E. rectale RNA-guided endonuclease knowns as Mad7 (SEQ ID NO: 2) that has been codon-optimized to Bacillus licheniformis is provided as SEQ ID NO: 1. However, a person skilled in the art will realize that expression of this parent RNA-guided endonuclease as well as temperature-sensitive variants thereof in a host cell different from Bacillus licheniformis may require that the DNA sequence is codon-optimized to said host cell. Thus, in a preferred embodiment, the temperature-sensitive variants of the invention are encoded by a polynucleotide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the polynucleotide sequence of SEQ ID NO: 1.

In an aspect, the present invention relates to temperature-sensitive variants of an RNA-guided endonuclease comprising an amino acid alteration at one or more (e.g., several) positions corresponding to positions 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 699, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1094, 1101, 1118, 1127, 1128, 1163, 1167, 1196, 1197, 1200, 1209, 1210, 1212, and 1213 of SEQ ID NO: 2, wherein each alteration is independently a substitution, insertion or deletion, and wherein the variants have a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2.

In an embodiment, the number of alterations in the variants of the present invention is 1-25, e.g., 1-20, 1-15, 1-10, and 1-5, such as 1, 2, 3, 4, 5, 6, 7, 8, 9, 10, 15, or 20 alterations.

In an embodiment, the variants comprise a substitution at one or more (e.g., several) positions corresponding to positions 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 699, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1094, 1101, 1118, 1127, 1128, 1163, 1167, 1196, 1197, 1200, 1209, 1210, 1212, and 1213 of SEQ ID NO: 2.

In an embodiment, the variants comprise a deletion at one or more (e.g., several) positions corresponding to positions 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 699, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1094, 1101, 1118, 1127, 1128, 1163, 1167, 1196, 1197, 1200, 1209, 1210, 1212, and 1213 of SEQ ID NO: 2.

In an embodiment, the variants comprise an insertion at one or more (e.g., several) positions corresponding to positions 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 699, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1094, 1101, 1118, 1127, 1128, 1163, 1167, 1196, 1197, 1200, 1209, 1210, 1212, and 1213 of SEQ ID NO: 2.

Preferably, the at least one alteration occurs in a position within the sequence of the RNA-guided endonuclease, i.e., the at least one alteration is not an N- or C-terminal extension, insertion, or fusion.

The temperature-sensitive variants of the invention comprise at least one alteration in a position important for stability of the RNA-guided endonuclease or for stability of a complex formed between the RNA-guided endonuclease, one or more gRNA, and/or one or more DNA target sequence. Such positions include, but are not limited to, positions corresponding to 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.

Preferably, the at least one alteration occur in a position important for stability of the RNA-guided endonuclease. Such positions include, but are not limited to, positions corresponding to 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 709, 713, 715, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.

Preferably, the at least one alteration occur in a position important for stability of a complex formed between the RNA-guided endonuclease and one or more gRNA. Such positions include, but are not limited to, positions corresponding to 707, 708, 709, 723, 833, 834, and 836 of SEQ ID NO: 2.

Preferably, the at least one alteration occur in a position important for stability of a complex formed between the RNA-guide endonuclease and one or more DNA target sequence. Such positions include, but are not limited to, positions corresponding to 159, 165, 294, 535, 590, 594, 649, 927, 1118, 1127, 1128, 1163, and 1167 of SEQ ID NO: 2.

Preferably, the at least one alteration occur in a position important for stability of the RNA-guide endonuclease and/or for stability of a complex formed between the RNA-guided endonuclease and one or more gRNA. Such positions include, but are not limited to, positions corresponding to 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 707, 708, 709, 713, 715, 723, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.

Preferably, the at least one alteration occur in a position important for stability of the RNA-guided endonuclease and/or for stability of a complex formed between the RNA-guided endonuclease and one or more DNA target sequence. Such positions include, but are not limited to, positions corresponding to 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 709, 713, 715, 732, 734, 738, 740, 747, 756, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.

Preferably, the at least one alteration occur in a position important for stability of a complex formed between the RNA-guided endonuclease and one or more gRNA and/or for stability of a complex formed between the RNA-guided endonuclease and one or more DNA target sequence. Such positions include, but are not limited to, positions corresponding to 159, 165, 294, 535, 590, 594, 649, 707, 708, 709, 723, 833, 834, 836, 927, 1118, 1127, 1128, 1163, and 1167 of SEQ ID NO: 2.

The at least one alteration occurring in a position important for stability of the RNA-guided endonuclease and/or or for stability of a complex formed between the RNA-guided endonuclease, one or more gRNA, and/or one or more DNA target sequence may be a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, R159K, R159H, R159Q, R159N, F164S, F164A, F164G, F164Y, F164L, K165R, K165H, K165Q, K165N, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, 0288A, C288G, Y294F, Y294Q, Y294K, Y294H, Y294N, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, K535R, K535H, K535Q, K535N, 1554S, 1554A, 1554G, 1554L, P574L, P574S, P574A, P574G, P574Q, P586L, P586S, P586A, P586G, P586Q, P588L, P588S, P588A, P588G, P588Q, K590R, K590H, K590Q, K590N, K594R, K594H, K594Q, K594N, K649R, K649H, K649Q, K649N, D681H, D681S, D681A, D681G, D681L, W682S, W682A, W682G, W682L, N707S, N707A, N707G, N707L, D708S, D708A, D708G, D708L, F709Y, F709Q, F709K, F709H, F709N, L713S, L713A, L713G, L713N, L715S, L715A, L715G, L715N, Y723F, Y723Q, Y723K, Y723H, Y723N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, R833K, R833Q, Y834F, Y834Q, Y834K, Y834H, Y834N, Y836F, Y836Q, Y836K, Y836H, Y836N, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, W927F, W927Q, W927K, W927H, W927N, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, R1118K, R1118H, R1118N, D1127N, D1127S, D1127A, T1128S, T1128V, T1128A, F1163Y, F1163Q, F1163K, F1163H, F1163N, R1167K, R1167H, R1167N P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.

Preferably, the at least one alteration is a substitution occurring in a position important for stability of the RNA-guided endonuclease and is selected from the group consisting of F34S, F34N, F34A, F34L, R51K R51Q, R51S, R51A, R51G, R51L, L54S, L54A, L54G, L54Y, I57S, 157A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, 0288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.

Preferably, the at least one alteration is a substitution occurring in a position important for stability of a complex formed between the RNA-guided endonuclease and one or more gRNA and is selected from the group consisting of N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, and Y836N.

Preferably, the at least one alteration is a substitution occurring in a position important for stability of a complex formed between the RNA-guided endonuclease and one or more DNA target sequence and is selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.

Preferably, the at least one alteration is a substitution occurring in a position important for stability of the RNA-guided endonuclease and/or for stability of a complex formed between the RNA-guided endonuclease and one or more gRNA and is selected from the group consisting of F34S, F34N, F34A, F34L, R51K R51Q, R51S, R51A, R51G, R51L, L54S, L54A, L54G, L54Y, 157S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, N707S, N707A, N707G, N707L, D708S, D708A, D708G, D708L, F709Y, F709Q, F709K, F709H, F709N, L713S, L713A, L713G, L713N, L715S, L715A, L715G, L715N, Y723F, Y723Q, Y723K, Y723H, Y723N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, R833K, R833Q, Y834F, Y834Q, Y834K, Y834H, Y834N, Y836F, Y836Q, Y836K, Y836H, Y836N, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.

Preferably, the at least one alteration is a substitution occurring in a position important for stability of the RNA-guided endonuclease and/or for stability of a complex formed between the RNA-guided endonuclease and one or more DNA target sequence and is selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, R159K, R159H, R159Q, R159N, F164S, F164A, F164G, F164Y, F164L, K165R, K165H, K165Q, K165N, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, Y294F, Y294Q, Y294K, Y294H, Y294N, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, K535R, K535H, K535Q, K535N, 1554S, 1554A, 1554G, 1554L, P574L, P574S, P574A, P574G, P574Q, P586L, P586S, P586A, P586G, P586Q, P588L, P588S, P588A, P588G, P588Q, K590R, K590H, K590Q, K590N, K594R, K594H, K594Q, K594N, K649R, K649H, K649Q, K649N, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, W927F, W927Q, W927K, W927H, W927N, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, R1118K, R1118H, R1118N, D1127N, D1127S, D1127A, T1128S, T1128V, T1128A, F1163Y, F1163Q, F1163K, F1163H, F1163N, R1167K, R1167H, R1167N, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.

Preferably, the at least one alteration is a substitution occurring in a position important for stability of a complex formed between the RNA-guided endonuclease and one or more gRNA and/or for stability of a complex formed between the RNA-guided endonuclease and one or more DNA target sequence and is selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, Y836N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.

Preferably, the at least one alteration is a substitution selected from the group consisting of 157S, M58S, L70S, L132P, G220D, L520A, F522Y, W531A, L669P, D708Y, N732S, K734N, L738A, E897S, Q898A, F901A, C1029A, F1031S, P1043L, and D1213N.

Preferably, the at least one alteration is selected from the group consisting of W531A and P1043L; L699P; N732S, K734N, L738A, and D1213N; L70S and D708Y; L132P; C1029A and F1031S; N732S, K734N, and L738A; L520A, F522Y, E897S, Q898A, and F901A; 157S, M58S, N732S, K734N, and L738A; N732S, K734N, L738A, E897S, Q898A, and F901A; and G220D, N732S, K734N, and L738A.

Preferably the at least one alteration include 157S.

Preferably the at least one alteration include M58S.

Preferably the at least one alteration include L70S.

Preferably the at least one alteration include L132P.

Preferably the at least one alteration include G220D.

Preferably the at least one alteration include L520A.

Preferably the at least one alteration include F522Y.

Preferably the at least one alteration include W531A.

Preferably the at least one alteration include L669P.

Preferably the at least one alteration include D708Y.

Preferably the at least one alteration include N732S.

Preferably the at least one alteration include K734N.

Preferably the at least one alteration include L738A.

Preferably the at least one alteration include E897S.

Preferably the at least one alteration include Q898A.

Preferably the at least one alteration include F901A.

Preferably the at least one alteration include C1029A.

Preferably the at least one alteration include F1031S.

Preferably the at least one alteration P1043L.

Preferably the at least one alteration include D1213N.

Preferably the at least one alteration include W531A+P1043L.

Preferably the at least one alteration include N732S+K734N+L738A+D1213N.

Preferably the at least one alteration include L70S+D708Y.

Preferably the at least one alteration include C1029A+F1031S.

Preferably the at least one alteration include N732S+K734N+L738A.

Preferably the at least one alteration include; L520A+F522Y+E897S+Q898A+F901A.

Preferably the at least one alteration include 157S+M58S+N732S+K734N+L738A.

Preferably the at least one alteration include N732S+K734N+L738A+E897S+Q898A+F901A.

Preferably the at least one alteration include G220D+N732S+K734N+L738A.

Preferably the at least one alteration include 157S+M58S.

Preferably the at least one alteration include F65A+L70S.

Preferably the at least one alteration include E194N+F197S.

Preferably the at least one alteration include K455N+A451N+E448S.

Preferably the at least one alteration include L520A+N521A.

Preferably the at least one alteration include L520A+N521A+P525G.

Preferably the at least one alteration include L520A+F522Y.

Preferably the at least one alteration include P525G+W531A.

Preferably the at least one alteration include P574Q+P756G.

Preferably the at least one alteration include P586G+P588S.

Preferably the at least one alteration include D681H+W682A.

Preferably the at least one alteration include N707A+K709N+L713A+L715A.

Preferably the at least one alteration include N732S+K734N+L738A.

Preferably the at least one alteration include L740A+L747S.

Preferably the at least one alteration include 1876A+A877H+R878S.

Preferably the at least one alteration include 1876Y+A877L+R878Q.

Preferably the at least one alteration include E897S+Q898A+F901A.

Preferably the at least one alteration include E929S+1930S.

Preferably the at least one alteration include E929S+1930S+K932N.

Preferably the at least one alteration include N1004A+Y1011A.

Preferably the at least one alteration include N1004K+Y1011H.

Preferably the at least one alteration include C1029A+F1031S.

Preferably the at least one alteration include P1196G+K1197N+D1200S.

The temperature-sensitive variants of the invention are characterized by a permissive temperature or temperature range, where the variant behaves as the wildtype protein and is able to form a complex with one or more gRNA and one or more DNA target sequences of interest, and by a restrictive temperature or temperature range, where the variant adopts the mutant phenotype and is unable to form a complex with one or more gRNA and one or more DNA target sequence of interest. In most cases, the restrictive temperature or temperature range is higher than the permissive temperature or temperature range.

The permissive and restrictive temperatures or temperature ranges depend on the temperature-sensitivity of a given variant of the invention and the temperature requirements of a given host cell. In general, the permissive and restrictive temperature are in the range from 25° C. to 45° C. Preferably, the permissive and restrictive temperature or temperature range should be separated by at least 1° C. Alternatively, the permissive and restrictive temperature ranges may overlap.

Depending on the host cell, the permissive temperature may be at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.

The permissive temperature may also be a temperature range selected from 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.

The permissive temperature may also be a temperature range selected from 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.

The permissive temperature may also be a temperature range selected from 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.

The permissive temperature may also be a temperature range selected from 25-31° C., 26-32° C., 27-33° C., 28-34° C., 29-35° C., 30-36° C., 31-37° C., 32-38° C., 33-39° C., 34-40° C., 35-41° C., 36-42° C., 37-43° C., 38-44° C., and 39-45° C.

The permissive temperature may also be a temperature range selected from 25-32° C., 26-33° C., 27-34° C., 28-35° C., 29-36° C., 30-37° C., 31-38° C., 32-39° C., 33-40° C., 34-41° C., 35-42° C., 36-43° C., 37-44° C., and 38-45° C.

The permissive temperature may also be a temperature range selected from 25-33° C., 26-34° C., 27-35° C., 28-36° C., 29-37° C., 30-38° C., 31-39° C., 32-40° C., 33-41° C., 34-42° C., 35-43° C., 36-44° C., and 37-45° C.

The permissive temperature may also be a temperature range selected from 25-34° C., 26-35° C., 27-36° C., 28-37° C., 29-38° C., 30-39° C., 31-40° C., 32-41° C., 33-42° C., 34-43° C., 35-44° C., and 36-45° C.

The permissive temperature may also be a temperature range selected from 25-35° C., 26-36° C., 27-37° C., 28-38° C., 29-39° C., 30-40° C., 31-41° C., 32-42° C., 33-43° C., 34-44° C., and 35-45° C.

The permissive temperature range may be selected from 25-28° C., 26-29° C., 27-30° C., 28-31° C., 30-32° C., 30-33° C., 29-32° C., 30-34° C., 29-33° C., 30-35° C., 29-34° C., 30-36° C., 28-34° C., 31-35° C., 28-35° C., 26-34° C., and 27-34° C.

Preferably, the permissive temperature range is selected from 25-27° C., 26-28° C., 27-30° C., 28-30° C., 29-31° C. 30-32° C., 29.5-32.5° C., 29-33° C., 28.5-33.5° C., 28-34° C., 27.5-34.5° C., 27-35° C., 26.5-35.5° C., and 26-36° C.

Preferably, the permissive temperature range is selected from 26-30° C., 27-31° C., 28-32° C., 29-33° C., 28.5-33.5° C., 28-34° C., 27.5-34.5° C., and 27-35° C.

Preferably, the permissive temperature range is selected from 26-32° C., 27-33° C., 28-34° C., and 29-35° C.

In a preferred aspect, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.

Depending on the host cell, the restrictive temperature may be at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.

The restrictive temperature may also be a temperature range selected from 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.

The restrictive temperature may also be a temperature range selected from 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.

The restrictive temperature may also be a temperature range selected from 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.

The restrictive temperature may also be a temperature range selected from 25-31° C., 26-32° C., 27-33° C., 28-34° C., 29-35° C., 30-36° C., 31-37° C., 32-38° C., 33-39° C., 34-40° C., 35-41° C., 36-42° C., 37-43° C., 38-44° C., and 39-45° C.

The restrictive temperature may also be a temperature range selected from 25-32° C., 26-33° C., 27-34° C., 28-35° C., 29-36° C., 30-37° C., 31-38° C., 32-39° C., 33-40° C., 34-41° C., 35-42° C., 36-43° C., 37-44° C., and 38-45° C.

The restrictive temperature may also be a temperature range selected from 25-33° C., 26-34° C., 27-35° C., 28-36° C., 29-37° C., 30-38° C., 31-39° C., 32-40° C., 33-41° C., 34-42° C., 35-43° C., 36-44° C., and 37-45° C.

The restrictive temperature may also be a temperature range selected from 25-34° C., 26-35° C., 27-36° C., 28-37° C., 29-38° C., 30-39° C., 31-40° C., 32-41° C., 33-42° C., 34-43° C., 35-44° C., and 36-45° C.

The restrictive temperature may also be a temperature range selected from 25-35° C., 26-36° C., 27-37° C., 28-38° C., 29-39° C., 30-40° C., 31-41° C., 32-42° C., 33-43° C., 34-44° C., and 35-45° C.

Preferably, the restrictive temperature range is selected from the group consisting of 38-40° C., 38-41° C., 37-40° C., 38-42° C., 37-41° C., 37-42° C., 36-40° C., 36-41° C., 42 36-42° C., 35-43° C., 34-44° C., 33-45° C., 25-43° C.

Preferably, the restrictive temperature range is selected from 39-40° C., 38.5-41.5° C., 38-42° C., 37.5-42° C., 38-42.5° C., 37-42° C., 37-42.5° C., 36.5-41.5° C., 36.5-42° C., and 36-43° C.

Preferably, the restrictive temperature range is selected from 38° C., 37.5-42° C., 38-42.5° C., 36.5-41.5° C., 36.5-42° C., 37-42° C., 37-42.5° C., 36-43° C., and 36-44° C.

Preferably, the restrictive temperature range is selected from 36.5-41.5° C., 36.5-42° C., 37-42° C., and 37-42.5° C.

In a preferred aspect, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.

In an aspect, the variant of the invention comprises or consists of an alteration, preferably a substitution, at a position corresponding to position 57 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 57 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another embodiment, the variant comprises or consists of the substitution 157S of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 58 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 58 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another embodiment, the variant comprises or consists of the substitution M58S of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 70 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 70 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another embodiment, the variant comprises or consists of the substitution L70S of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.

Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 132 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 132 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro. In another embodiment, the variant comprises or consists of the substitution L132P of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 220 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 220 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another embodiment, the variant comprises or consists of the substitution G220D of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 520 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 520 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another embodiment, the variant comprises or consists of the substitution L520A of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 522 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 522 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another embodiment, the variant comprises or consists of the substitution F522Y of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 531 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 531 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another embodiment, the variant comprises or consists of the substitution W531A of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 669 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 669 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro. In another embodiment, the variant comprises or consists of the substitution L669P of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 708 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 708 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another embodiment, the variant comprises or consists of the substitution D708Y of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 732 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 732 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another embodiment, the variant comprises or consists of the substitution N732S of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 734 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 734 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asn. In another embodiment, the variant comprises or consists of the substitution K734N of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 738 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 738 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another embodiment, the variant comprises or consists of the substitution L738L of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 897 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 897 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another embodiment, the variant comprises or consists of the substitution E897S of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 898 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 898 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another embodiment, the variant comprises or consists of the substitution Q898A of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 901 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 901 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another embodiment, the variant comprises or consists of the substitution F901A of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 1029 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 1029 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another embodiment, the variant comprises or consists of the substitution C1029A of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is higher than the permissive temperature. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 1031 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 1031 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another embodiment, the variant comprises or consists of the substitution F1031S of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 1043 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 1043 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another embodiment, the variant comprises or consists of the substitution P1043L of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

In an aspect, the variant of the invention comprises or consists of an alteration, preferably a substitution, at a position corresponding to position 1213 of SEQ ID NO: 2, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of SEQ ID NO: 2. In an embodiment, the amino acid at a position corresponding to position 1213 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asn. In another embodiment, the variant comprises or consists of the substitution D1213N of SEQ ID NO: 2. In a preferred embodiment, the variant is a nuclease-null variant; preferably said variant further comprises an alteration of an amino acid corresponding to position 877 of SEQ ID NO: 2; more preferably said variant comprises a substitution of aspartic acid for alanine, D877A. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is higher than the permissive temperature. Preferably, the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below. Preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C. Preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above. Preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C. Preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C. Preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C. Preferably, the restrictive temperature is higher than the permissive temperature.

### Preparation of Temperature-Sensitive Variants

The present invention also relates to methods for obtaining a temperature-sensitive variant of an RNA-guided endonuclease, said method comprising: (a) introducing into a parent RNA-guided endonuclease at least one alteration at one or more (e.g., several) positions important for stability of the RNA-guided endonuclease or for stability of a complex formed between the RNA-guided endonuclease, one or more gRNA, and one or more DNA target sequence, and, optionally, (b) recovering the variant.

The variants of the invention can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to polynucleotides encoding temperature-sensitive variants of an RNA-guided endonuclease. In a preferred embodiment, the polynucleotide encoding variants of the inventions has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 1.

The present invention also relates to isolated polynucleotides encoding a temperature-sensitive variant of an RNA-guided endonuclease. The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Eubacterium, preferably E. rectale, or a related organism and thus, for example, may be an allelic or species variant of the polypeptide encoding region of the polynucleotide.

Modification of a polynucleotide encoding a temperature-sensitive variant of the present invention may be necessary for synthesizing polypeptides substantially similar to such variant. The term “substantially similar” to the polypeptide refers to non-naturally occurring forms of the polypeptide.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a nucleic acid construct or expression vector may be desirable or necessary depending on the construct or vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alphaamylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CU P1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene. Examples of suitable mRNA stabilizer regions are obtained from a Bacillus thuringiensis ctyIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883. More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells comprising a variant, polynucleotide, nucleic acid construct, and/or expression vector of the present invention. A polynucleotide of the present invention is operably linked to one or more control sequences that direct the production of a variant of the present invention. A nucleic acid construct or expression vector comprising a polynucleotide is introduced into a host cell so that the construct or vector is maintained as a chromosomal integrant or as a self-replicating extra-chromosomal vector as described earlier.

The term “host cell” encompasses any progeny of a parent cell that is not identical to the parent cell due to mutations that occur during replication. The choice of a host cell will to a large extent depend upon the gene encoding the variant and its source.

The host cell may be any cell useful in the context of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells. In a preferred embodiment, the bacterial host cell is a B. subtilis or B. licheniformis cell.

The bacterial host cell may also be any Streptococcus cell including, but not limited to, Streptococcus equisimilis, Streptococcus pyogenes, Streptococcus uberis, and Streptococcus equi subsp. zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The bacterial host cell may also be any Lactobacillus cell including, but not limited to, Lactobacillus acidophilus, Lactobacillus amylovorus, Lactobacillus brevis, Lactobacillus (para)casei, Lactobacillus cellobiosus, Lactobacillus crispatus, Lactobacillus curvatus, Lactobacillus delbrueckii subsp. bulgaricus, L. delbrueckii subsp. lactis, Lactobacillus fermentum, Lactobacillus gallinarum, Lactobacillus gasseri, Lactobacillus helveticus, Lactobacillus johnsonii, Lactobacillus plantarum, Lactobacillus reuteri, Lactobacillus rhamnosus, and Lactobacillus salivarius cell.

The bacterial host cell may also be any Lactococcus cell including, but not limited to, Lactococcus chungangensis, Lactococcus formosensis, Lactococcus fujiensis, Lactococcus garvieae, Lactococcus lactis, Lactococcus piscium, Lactococcus plantarum, Lactococcus raffinolactis, and Lactococcus taiwanensis.

The bacterial host cell may also be any Eschericia cell. In a preferred embodiment, the bacterial host cell is an E. coli cell.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and DavidoffAbelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell. For eukaryotic host cells, the temperature-sensitive variants of the invention must comprise at least one nuclear localization sequence (NLS) fused to the variant in order to ensure its localization in the nucleus of the cell; preferably the Simian virus 40 (SV40) T antigen nuclear localization signal (NLS) is fused on the N- and/or C-terminus of the variant.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as S. cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longi brachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; I to et al., 1983, J. Bacteriol. 153: 163; and Hinnen et al., 1978, Proc. Natl. Acad. Sci. USA 75: 1920.

### Methods for Inducing or Repressing Expression

The present invention also relates to methods for inducing or repressing expression of one or more DNA target sequence of interest. Preferably, the one or more DNA target sequence is one or more genome target sequence, i.e., a DNA sequence that is part of the genome of an organism. The skilled person will understand that a DNA target sequence should be a DNA target sequence, i.e., a DNA target sequence that is complementary to an capable of hybridizing to a gRNA suitable for directing the binding and/or nuclease activity of the variants of the invention.

In a preferred aspect, the present invention relates to a method of repressing one or more DNA target sequence, the method comprising the steps of:

a) providing a host cell according to claim 18, said host cell further comprising one or more gRNA and one or more DNA target sequence;

b) cultivating the host cell at a restrictive temperature of the variant and under conditions conducive for expression of the variant, wherein the one or more DNA target sequence is expressed; and subsequently

c) decreasing the temperature to a permissive temperature of the variant and cultivating the host cell, whereby a complex is formed in the host cell between the variant, the one or more gRNA and the one or more DNA target sequence, and whereby expression of the one or more DNA target sequence is repressed.

The method may also comprise the additional step of:

d) increasing the temperature to a restrictive temperature of the variant and cultivating the host cell, whereby the formed complex dissociates and expression of the one or more DNA target sequence is induced.

Preferably, wherein the permissive temperature is at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C., the restrictive temperature is at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C., and the restrictive temperature is higher than the permissive temperature.

In another preferred aspect, the present invention relates to a method of repressing one or more DNA target sequence, the method comprising the steps of:

a) providing a host cell according to claim 18, said host cell further comprising one or more gRNA and one or more DNA target sequence;

b) cultivating the host cell at a restrictive temperature of the variant and under conditions conducive for expression of the variant, wherein the one or more DNA target sequence is expressed; and subsequently

c) decreasing the temperature to a permissive temperature of the variant and cultivating the host cell, whereby a complex is formed in the host cell between the variant, the one or more gRNA and the one or more DNA target sequence, and whereby expression of the one or more DNA target sequence is repressed.

The method may also comprise the additional step of:

d) increasing the temperature to a restrictive temperature of the variant and cultivating the host cell, whereby the formed complex dissociates and expression of the one or more DNA target sequence is induced.

Preferably, the permissive temperature is at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C., the restrictive temperature is at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C., and the restrictive temperature is higher than the permissive temperature.

For methods of inducing or repressing expression, the host cells is preferably a Bacillus host cell; preferably the host cell is a Bacillus cell selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cell; more preferably the host cell is a Bacillus licheniformis cell.

For Bacillus host cells, the permissive temperature is a range selected from 30-32° C., 30-33° C., 29-32° C., 30-34° C., 29-33° C., 30-35° C., 29-34° C., 30-36° C., 28-34° C., 31-35° C., 28-35° C., 26-34° C., and 27-34° C. Preferably, the permissive temperature range is selected from 30-32° C., 29.5-32.5° C., 29-33° C., 28.5-33.5° C., 28-34° C., 27.5-34.5° C., 27-35° C., 26.5-35.5° C., and 26-36° C. More preferably, the permissive temperature range is selected from 29-33° C., 28.5-33.5° C., 28-34° C., 27.5-34.5° C., and 27-35° C. Even more preferably, the permissive temperature range is selected from 28.5-33.5° C., 28-34° C., and 27.5-34.5° C. Most preferably, the permissive temperature range is 28-34° C.

For Bacillus host cells, the restrictive temperature is a range selected from 38-40° C., 38-41° C., 37-40° C., 38-42° C., 37-41° C., 37-42° C., 36-40° C., 36-41° C., 36-42° C., and 35-43° C. Preferably, the restrictive temperature range is selected from 39-40° C., 38.5-41.5° C., 38-42° C., 37.5-42° C., 38-42.5° C., 36.5-41.5° C., 36.5-42° C., 37-42° C., 37-42.5° C., and 36-43° C. More preferably, the restrictive temperature range is selected from 38-42° C., 37.5-42° C., 38-42.5° C., 36.5-41.5° C., 36.5-42° C., 37-42° C., 37-42.5° C., 36-43° C., and 36-44° C. Even more preferably, the restrictive temperature range is selected from 36.5-41.5° C., 36.5-42° C., 37-42° C., and 37-42.5° C. Most preferably, the restrictive temperature range is 37-42° C.,

In a preferred embodiment, the permissive temperature is 28-34° C., and the restrictive temperature is 37-42° C.

### Guide-RNA

The gRNA in CRISPR genome editing constitutes the re-programmable part that makes the system so versatile. In the natural S. pyogenes system, the gRNA is actually a complex of two RNA polynucleotides, a first crRNA containing about 20 nucleotides that determine the specificity of the RNA-guided endonuclease known as Cas9 and the tracr RNA which hybridizes to the crRNA to form an RNA complex that interacts with Cas9 (see Jinek et al., 2012, A programmable dual-RNA-guided DNA endonuclease in adaptive bacterial immunity, Science 337: 816-821). The terms crRNA and tracrRNA are used interchangeably with the terms tracrmate RNA and tracr RNA herein.

Since the discovery of the CRISPR-Cas9 system single polynucleotide gRNAs have been developed and successfully applied just as effectively as the natural two-part gRNA complex.

In a preferred embodiment, the one or more gRNA is a single gRNA or RNA complex comprising a first RNA comprising 20 or more nucleotides that are at least 85% complementary to and capable of hybridizing to the one or more DNA target sequence; preferably the 20 or more nucleotides are at least 90%, 95%, 97%, 98%, 99% or even 100% complementary to and capable of hybridizing to the one or more DNA target sequence.

In another preferred embodiment, a host cell of the invention comprises a single gRNA comprising the first and second RNAs in the form of a single polynucleotide and wherein the tracr mate sequence and the tracr sequence form a stem-loop structure when hybridized with each other.

### DNA Target Sequence

The one or more DNA target sequence should be a DNA target sequence, i.e., a DNA target sequence that is complementary to an capable of hybridizing to a gRNA suitable for directing the binding and/or nuclease activity of a variants of the invention.

Preferably, the one or more DNA target sequence is at least 20 nucleotides in length in order to allow its hybridization to the corresponding at least 20 nucleotide sequence of the one or more gRNA. The one or more DNA target sequence can be located anywhere in the genome but will often be within a coding sequence or open reading frame.

In a preferred embodiment, the one or more DNA target sequence comprises a polynucleotide comprising 20 or more nucleotides that are at least 85% complementary to and capable of hybridizing to the one or more gRNA; preferably the 20 or more nucleotides are at least 90%, 95%, 97%, 98%, 99% or even 100% complementary to and capable of hybridizing to the one or more gRNA.

The one or more DNA target sequence should be flanked by a functional PAM sequence for a variant of the invention. For an overview of PAM sequences, see, for example, Shah et al., 2013, Protospacer recognition motifs, RNA Biol. 10(5): 891-899.

Preferably, the one or more DNA target sequence is comprised in an open reading frame encoding a polypeptide or in a promoter region. Also preferably, the one or more DNA target sequence encode one or more enzyme selected from the group consisting of hydrolase, isomerase, ligase, lyase, oxidoreductase, or a transferase; preferably the one or more enzyme is an alpha-amylase, alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucano-transferase, glucoamylase, invertase, laccase, lipase, mannosidase, mutanase, oxidase, pectinolytic enzyme, peroxidase, phosphodiesterase, phytase, polyphenoloxidase, proteolytic enzyme, ribonuclease, transglutaminase, and xylanase.

Preferably, the one or more DNA target sequence encodes green fluorescent protein, a fragment of a variant thereof.

Preferably, if the host cel of the invention I is a Bacillus host cell, the one or more DNA target sequence to be repressed comprises the mecA and/or the yjbH gene or homologues thereof. Other preferred DNA target sequences of interest to be repressed comprise protease-encoding genes, especially cytosolic, secreted or membrane-bound proteases that, if expressed, may degrade a recombinantly produced polypeptide.

### Methods of Production of Temperature-Sensitive Variants

The present invention also relates to methods of producing a variant of the invention, comprising: (a) cultivating a host cell of the present invention under conditions suitable for expression of the variant; and (b) recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid-state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the such variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

### Catalytically Inactive RNA-Guided Endonuclease

In some aspect of the present invention, the temperature-sensitive variants of the invention are catalytically inactive. These variants are based on a parent RNA-guided endonuclease that is catalytically inactive, i.e., it can bind to its target DNA sequence, but cannot introduce any breaks in the target DNA sequence.

In one aspect, the present invention relates to a nuclease-null variant of an RNA-guided endonuclease, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 2, and wherein the variant comprises an alteration of an amino acid at a position corresponding to position 877 of SEQ ID NO: 2. In an embodiment, the amino acid at a position corresponding to position 877 of SEQ ID NO: 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another embodiment, the variant comprises or consists of the substitution D877A of SEQ ID NO: 2.

The present invention is further described by the following examples that should not be construed as limiting to the scope of the invention.

### Examples

### Materials and Methods

### Materials

Chemicals used as buffers and substrates were commercial products of at least reagent grade.

PCR amplifications were performed using standard textbook procedures, employing a commercial thermocycler and either Ready-To-Go PCR beads, Phusion polymerase, or REDTAQ polymerase from commercial suppliers.

LB agar: See EP 0 506 780.

LBPSG agar plates contains LB agar supplemented with phosphate (0.01 M K3PO4), glucose (0.4%), and starch (0.5%); See EP 0 805 867 B1.

TY (liquid broth medium; See WO 94/14968, p. 16.

Oligonucleotide primers were obtained from DNA technology, Aarhus, Denmark or Sigma-Aldrich Denmark. DNA manipulations (plasmid and genomic DNA preparation, restriction digestion, purification, ligation, DNA sequencing) was performed using standard textbook procedures with commercially available kits and reagents.

Ligation mixtures were in some cases amplified in an isothermal rolling circle amplification reaction, using the TempliPhi kit from GE Healthcare.

DNA was introduced into B. subtilis rendered naturally competent, either using a twostep procedure (Yasbin et al., 1975, J. Bacteriol. 121: 296-304), or a one-step procedure, in which cell material from an agar plate was resuspended in Spizisen 1 medium (WO 2014/052630), 12 ml shaken at 200 rpm for approx. 4 hours at 37° C., DNA added to 400 microliter aliquots, and these further shaken 150 rpm for 1 hour at the desired temperature before plating on selective agar plates.

DNA was introduced into B. licheniformis by conjugation from B. subtilis, essentially as previously described (EP2029732 B1), using a modified B. subtilis donor strain BKQ2527, containing pLS20, wherein the methylase gene M.b1i190411 (US20130177942) is expressed from a triple promoter at the amyE locus, the pBC16-derived orf beta and the B. subtilis comS gene (and a kanamycin resistance gene) are expressed from a triple promoter at the alr locus (making the strain D-alanine requiring), and the B. subtilis comK gene expressed from a mannose inducible promoter in the xylA locus.

Bacillus subtilis JA1343: JA1343 is a sporulation negative derivative of PL1801 (WO 2005042750). Part of the gene spollAC has been deleted to obtain the sporulation negative phenotype.

All the constructions described in the examples were assembled from synthetic DNA fragments ordered from GeneArt— ThermoFisher Scientific. The fragments were assembled by sequence overlap extension (SOE) as described in the examples. For plasmid construction was mainly used Prolonged Overlap Extension PCR (POE-PCR), which generates multimeric plasmids, as previously described (You et al. 2012. Simple cloning via direct transformation of PCR product (DNA multimer) to Escherichia coli and Bacillus subtilis. Appl. Environ. Microbiol. 78(5): 1593-1595).

The temperature-sensitive plasmids used in this patent was incorporated into the genome of B. licheniformis by chromosomal integration and excision according to the method previously described (U.S. Pat. No. 5,843,720). B. licheniformis transformants containing plasmids were grown on LBPG selective medium with erythromycin at 50° C. to force integration of the vector at identical sequences to the chromosome. Desired integrants were chosen based on their ability to grow on LBPG+erythromycin selective medium at 50° C. Integrants were then grown without selection on LBPG plates at 34° C. to allow excision of the integrated plasmid. Cells were then grown in liquid LBPG medium at 37° C. for 6-8 hours. The cultures were then plated on LBPG plates and screened for erythromycin-sensitivity. The sensitive clones were checked for correct integration of the desired construct.

Genomic DNA was prepared from several erythromycin sensitive isolates above by using the commercially available QIAamp DNA Blood Kit from Qiagen.

### Standard Fed-Batch Cultivation Procedure

All growth media were sterilized by methods known in the art. Unless otherwise described, tap water was used. The ingredient concentrations referred to in the below recipes are before any inoculation.

First inoculum medium: SSB4 agar. Soy peptone SE50MK (DMV) 10 g/l; sucrose 10 g/l; di-sodiumhydrogenphosphate, 2H₂O 5 g/l; potassiumdihydrogenphosphate 2 g/l; citric acid 0.2 g/l; vitamins (Thiamin-hydrochlorid 1 1,4 mg/l; riboflavin 0.95 mg/l; nicotinic amide 7.8 mg/l; calcium D-pantothenate 9.5 mg/l; pyridoxal-HCl 1.9 mg/l; D-biotin 0.38 mg/l; folic acid 2.9 mg/l); trace metals (MnSO₄, H₂O 9.8 mg/l; FeSO₄, 7H₂O 39.3 mg/l; CuSO₄, 5H₂O 3.9 mg/l; ZnSO₄, 7H₂O 8.2 mg/l); Agar 25 g/l. Use of deionized water. pH adjusted to pH 7.3 to 7.4 with NaOH.

Transfer buffer. M-9 buffer (deionized water is used): Di-sodiumhydrogenphosphate, 2H₂O 8.8 g/l; potassiumdihydrogenphosphate 3 g/l; sodium chloride 4 g/l; magnesium sulphate, 7H₂O 0.2 g/l.

Inoculum shake flask medium (concentration is before inoculation): PRK-50: 10 g/l soy grits; di-sodiumhydrogenphosphate, 2H₂O 5 g/l; pH adjusted to 8.0 with NaOH/H₃PO₄ before sterilization.

Make-up medium (concentration is before inoculation): Tryptone (casein hydrolysate from Difco) 30 g/l; magnesium sulphate, 7H₂O 4 g/l; di-potassiumhydrogenphosphate 7 g/l; disodiumhydrogenphosphate, 2H₂O 7 g/l; di-ammoniumsulphate 4 g/l; potassiumsulphate 5 g/l; citric acid 0.78 g/l; vitamins (Thiamin-hydrochloride 34.2 mg/l; Riboflavin 2.8 mg/l; Nicotinic amide 23.3 mg/l; calcium D-pantothenate 28.4 mg/l; pyridoxal-HCl 5.7 mg/l; D-biotin 1.1 mg/l; folic acid 2.5 mg/l); trace metals (MnSO₄, H₂O 39.2 mg/l; FeSO₄, 7H₂O 157 mg/l; CuSO₄, 5H₂O 15.6 mg/l; ZnSO₄, 7H₂O 32.8 mg/l); Antifoam (SB2121) 1.25 ml/l; pH adjusted to 6.0 with NaOH/H₃PO₄ before sterilization.

Feed medium: Sucrose 708 g/l;

Inoculum steps: First the strain was grown on SSB-4 agar slants 1 day at 37° C. The agar was then washed with M-9 buffer, and the optical density (OD) at 650 nm of the resulting cell suspension was measured. The inoculum shake flask (PRK-50) was inoculated with an inoculum of OD (650 nm)×ml cell suspension=0.1. The shake flask was incubated at 37° C. at 300 rpm for 20 hr. The fermentation in the main fermentor (fermentation tank) was started by inoculating the main fermentor with the growing culture from the shake flask. The inoculated volume was 11% of the make-up medium (80 ml for 720 ml make-up media).

Standard lab fermentors were used equipped with a temperature control system, pH control with ammonia water and phosphoric acid, dissolved oxygen electrode to measure oxygen saturation through the entire fermentation.

Fermentation parameters: Temperature: 30-42° C.; The pH was kept between 6.8 and 7.2 using ammonia water and phosphoric acid; Control: 6.8 (ammonia water); 7.2 phosphoric acid;

Aeration: 1.5 liter/min/kg broth weight.

Agitation: 1500 rpm.

Feed strategy: 0 hr. 0.05 g/min/kg initial broth after inoculation; 8 hr. 0.156 g/min/kg initial broth after inoculation; End 0.156 g/min/kg initial broth after inoculation.

Experimental setup: The cultivation was run for five days with constant agitation, and the oxygen tension was followed on-line in this period. The different strains were compared side by side.

Measurements of amylase activities was performed by standard technique using AMYL kit from Roche Diagnostics GmbH/Hitachi, REF 11876473.

Measurements of GFP fluorescence was performed on full broth cell cultures. The culture was diluted and measured directly on a SpectraMax M2 from Molecular Devices.

### Strains

PL1801: This strain is the B. subtilis DN 1885 with disrupted aprE and nprE genes encoding the alkaline protease and neutral protease, respectively (Diderichsen, B., Wedsted, U., Hedegaard, L., Jensen, B. R., Sjøholm, C. (1990) Cloning of aldB, which encodes alpha-acetolactate decarboxylase, an exoenzyme from Bacillus brevis. J. Bacteriol., 172, 4315-4321).

JA1343: This strain is the B. subtilis PL1801 with a disrupted spollAC gene (sigF). The genotype is: aprE, nprE, amyE, spollAC.

PP2307: This B. subtilis strain is JA1343 with an expression cassette inserted into the pel gene holding P3 promoter driving comS and a kanamycin marker.

PP5625: This strain is the B. subtilis strain JA1343 with the gfp gene, gDNA(gfp) and spec marker inserted at the pel locus.

MOL3268: This strain is the B. subtilis strain PP5625 with the mad7d gene and cat marker inserted at the amyE locus.

AEB1517: This strain is a B. subtilis donor strain for conjugation of B. licheniformis as described in several patents (U.S. Pat. Nos. 5,695,976A, 5,733,753A, 5,843,720A, 5,882,888A, WO2006042548A1). The strain contains pLS20 and the methylase gene M.b1i190411 (US20130177942) expressed from a triple promoter at the amyE locus, the pBC16-derived orf beta and the B. subtilis comS gene (and a kanamycin resistance gene) are expressed from a triple promoter at the alr locus (making the strain D-alanine requiring).

BKQ2284: This strain is B. subtilis AEB1517 with an expression cassette inserted into the xylA locus holding a mannitol inducible promoter driving comK expression and a chloramphenicol marker.

BKQ2527: This B. subtilis strain is BKQ2284 with a deletion of the chloramphenicol marker.

SJ4671: This B. licheniformis strain has two copies of the amyL gene integrated at the original amyL locus on the chromosome. The two copies are inserted in opposite directions so that transcription of the two copies are antiparallel. The copies are spaced by approximately 2.5 kb originating from non-functional DNA of the B. subtilis chromosome (U.S. Pat. No. 6,100,063).

SJ6026: This B. licheniformis strain has four copies of the amyL gene integrated at the amyL, xyl and gnt loci.

MOL2173: This B. licheniformis strain has four copies of the amyL gene integrated at the amyL, xyl and gnt loci and one additional prsA gene inserted at the mprL locus.

MOL2212: This B. licheniformis strain is a rifampicin resistant isolate of MOL2173.

PP5007: This is B. licheniformis strain MOL2212 where the native catL gene is inactivated. The strain is chloramphenicol sensitive.

BKQ3716: This is B. subtilis strain PP5625 with the temperature sensitive mad7d gene variant TS1 and cat marker inserted at the pel locus.

BKQ3717: This is B. subtilis strain PP5625 with the temperature sensitive mad7d gene variant TS2 and cat marker inserted at the pel locus.

BKQ3718: This is B. subtilis strain PP5625 with the temperature sensitive mad7d gene variant TS3 and cat marker inserted at the pel locus.

BKQ3775: This is B. subtilis strain PP5625 with the temperature sensitive mad7d gene variant TS4 and cat marker inserted at the pel locus.

BKQ3776: This is B. subtilis strain PP5625 with the temperature sensitive mad7d gene variant TS5 and cat marker inserted at the pel locus.

BKQ3777: This is B. subtilis strain PP5625 with the temperature sensitive mad7d gene variant TS6 and cat marker inserted at the pel locus.

BKQ3778: This is B. subtilis strain PP5625 with the temperature sensitive mad7d gene variant TS7 and cat marker inserted at the pel locus.

BKQ3801: This is B. subtilis strain PP5625 with the temperature sensitive mad7d gene variant TS8 and cat marker inserted at the pel locus.

BKQ3803: This is B. subtilis strain PP5625 with the temperature sensitive mad7d gene variant TS9 and cat marker inserted at the pel locus.

BKQ3805: This is B. subtilis strain PP5625 with the temperature sensitive mad7d gene variant TS10 and cat marker inserted at the pel locus.

BKQ3809: This is B. subtilis strain PP5625 with the temperature sensitive mad7d gene variant TS11 and cat marker inserted at the pel locus.

BKQ3934: This is B. licheniformis strain PP5007 where an expression cassette holding the mad7d gene, gDNA(P4199) and cat is inserted at the forD locus.

BKQ3913: This is B. licheniformis strain PP5007 where an expression cassette holding the mad7d variant TS6, gDNA(P4199) and cat is inserted at the forD locus.

BKQ3917: This B. licheniformis strain is PP5007 where an expression cassette holding the mad7d variant TS7, gDNA(P4199) and cat is inserted at the forD locus.

BKQ3948: This B. licheniformis strain is PP5007 where an expression cassette holding the mad7d variant TS5, gDNA(P4199) and cat is inserted at the forD locus.

### Plasmids

pC194: Plasmid isolated from Staphylococcus aureus (Horinouchi and Weisblum, 1982).

pE194: Plasmid isolated from S. aureus (Horinouchi and Weisblum, 1982).

pUB110: Plasmid isolated from (McKenzie et al., 1986)

pBKQ3825: Plasmid with a temperature sensitive origin and erm gene coding for erythromycin resistance. The plasmid contains flanking regions of forD of B. licheniformis, which enables chromosomal integration in the forD locus. Between the flanking regions is inserted the mad7d gene, gDNA(P4199) expressed from the PamyQsc promoter and a cat gene conferring chloramphenicol resistance.

### Example 1. Construction of the B. licheniformis Host MOL2212

The full construction of MOL2212 was done in several consecutive steps by sequential plasmid integrations as described below. The plasmids for integrations were assembled by PCR amplifications of synthetic DNA. The purified PCR products were used in a subsequent PCR reaction to create a single plasmid using splice overlapping PCR (SOE) using the Phusion Hot Start DNA Polymerase system (Thermo Scientific) as follows. The PCR amplification reaction mixture contained 50 ng of each of the six gel purified PCR products and a thermocycler was used to assemble and amplify the plasmids. The resulting SOE product were used directly for transformation of B. subtilis host JA1343 to establish the plasmids which were later used as vehicles for transfer and integration of DNA into specific loci on the B. licheniformis chromosome.

The B. licheniformis strain SJ4671 (U.S. Pat. No. 6,100,063) was used as a host strain for insertion of additional copies of the alpha-amylase gene amyL. The SJ4671 strain already has two copies of the amyL gene integrated at the original amyL locus on the chromosome. The two copies are inserted in opposite directions so that transcription of the two copies are antiparallel. The copies are spaced by approximately 2.5 kb originating from non-functional DNA of the B. subtilis chromosome (FIG. 1 and SEQ ID NO:4).

The SJ4671 strain was conjugated with plasmids for further insertions of two more copies of the amyL gene; one copy inserted at the xyl locus (FIG. 2 and SEQ ID NO: 4), and one copy inserted at the gnt locus (FIG. 3 and SEQ ID NO: 5). This intermediate strain was named SJ6026 and contained four copies of the alpha-amylase gene, amyL, stably integrated into the chromosome of B. licheniformis.

The SJ6026 strain was further engineered by inserting an additional copy of the prsA gene which encodes the chaperone from B. licheniformis. Over-expression of the PrsA chaperone is described in literature to further increase alpha-amylase productivity. The additional prsA gene was inserted at the mprL locus. This insertion lead to over-expression of the PrsA chaperone from B. licheniformis and knock-out of the mprL product—metalloprotease (FIG. 4 and SEQ ID NO: 6). This four-copy amyL and two-copy prsA strain was named MOL2173.

The final strain, named MOL2212, is a derivative of MOL2173 where a spontaneous mutation in the rpoB gene was isolated as a rifampicin resistant strain.

### Example 2. Construction of Mad7d

Inactivation of the catalytic site for endonuclease activity in Mad7 was based on finding homologous sequences of the active site in the close relative FnCpf1 (Zetsche et al., 2015, Cell 163, 759-771). The paper describes that the RuvC-like domain of Cpf1 retains all of the catalytic residues of this family of endonucleases and is thus predicted to be an active endonuclease. One amino acid change in the RuvC-like domain FnCpf1 (D917A) is demonstrated to completely inactivate the nuclease activity. The corresponding conserved region was identified in Mad7 by a multiple alignment of a part of the protein to two other closely related proteins (alignment is obtained with MUSCLE algorithm). FIG. 18 shows a multiple alignment of a part of these protein is shown. The protein sequences are from the organisms; Lachnospiraceae bacterium (LbCpf1), Francisella tularensis (FnCpf1) and E. rectale (Mad7) and shows a region of identity in the RuvC1 region where the Asp (D) can be changed to Ala (A). The amino acid substitution D877A results in inactivation of the catalytic site for nuclease activity in Mad7. The amino acid change was introduced by two overlapping primers in a SOE reaction followed by cloning as described in Example 6.

### Example 3. Construction of the B. licheniformis Strains PP5007 and BKQ3934

The MOL2212 strain was used as a host strain for transformation of a plasmid for inactivation of the native catL gene. A clone was isolated as chloramphenicol sensitive and preserved as PP5007.

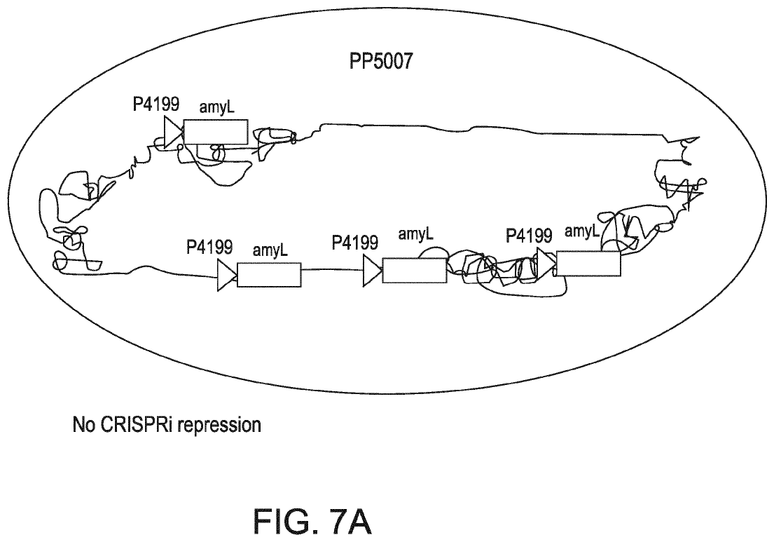

The PP5007 strain was further engineered by transformation of plasmid pBKQ3825 (FIG. 5, SEQ ID NO: 7), which integrates at the forD locus and inserts an expression cassette consisting of the mad7d gene expressed from the forD promoter, the gDNA(P4199) (SEQ ID NO: 12) transcribed from the PamyQsc promoter and the cat gene conferring chloramphenicol resistance (FIG. 6 and SEQ ID NO: 8). The gDNA(P4199) is transcribed into gRNA(P4199), which directs Mad7d to the P4199 promoter and inhibit transcription. All four amyL gene copies in the final strain BKQ3934 are expressed from the P4199 promoter, and the Mad7d-gRNA complex can potentially bind and inhibit alpha-amylase expression through strong interaction to the P4199 target. An illustration of the two B. licheniformis strains PP5007 and BKQ3934 is shown in FIG. 7. An illustration of the CRISPRi complex binding to the promoter region of P4199 is shown in FIG. 14b.

### Example 4. Alpha-Amylase Expression Using the PP5007 and BKQ3934 Strains—Temperature Shift

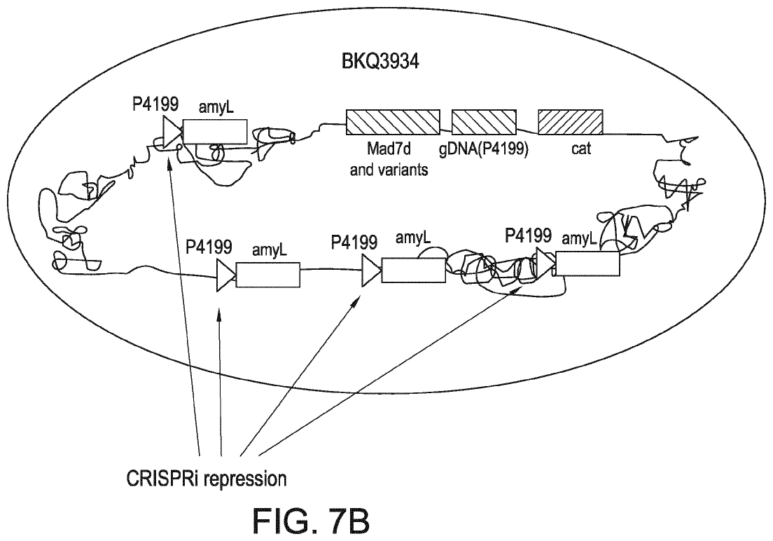

The B. licheniformis strains described in Example 3 was tested with respect to alphaamylase productivity at temperatures of 30° C. and 42° C. in fed-batch cultivations as described above. The PP5007 strain has full expression of the amylase from four copies with no inhibition from the Mad7d complex and can be used as a positive control for the BKQ3934, where the Mad7d complex is cloned and active. FIG. 8 clearly shows that the PP5007 strain has full expression of the amylase enzyme regardless of the cultivation temperature at 30° C. or 42° C. On the contrary, when strain BKQ3934 is cultivated at 30° C. or 42° C. the productivity of amylase is close to zero, which shows that the Mad7d complex formed by the gRNA(P4199), and the Mad7d can inhibit very efficiently the amylase expression from all four copies on the chromosome at temperature interval of 30° C. to 42° C.

When the temperature in the fed-batch fermentation of PP5007 was shifted from 30° C. to 42° C., a decrease in amylase expression was observed for the next 24-36 hours, which is probably related to a heat shock response. When the temperature was shifted from 42° C. to 30° C. a decrease in amylase expression was observed, which is expected as expression of this amylase is higher at 42° C. than at 30° C.

The experiments in this example shows that even at a relatively high temperature of 42° C., this is not sufficient to prohibit Mad7d complex formation and inhibition. Consequently, the Mad7d complex cannot be inactivated within physiologically compatible temperature for growth of mesophilic Bacillus organisms. With this information at hand we wanted to screen for destabilized site-directed variants of Mad7d, which had a suitable temperature induction profile than the current Mad7d protein. Screening for temperature-sensitive Mad7d protein therefore have two goals:
1) Obtaining de-stabilized Mad7d proteins which prohibit CRISPRi complex formation (Mad7d-gRNA) at a lower temperature than the wildtype Mad7d. This will allow a temperature range better suited for—in this case—the physiology of the Bacillus organism during cultivations. In the case of Bacillus, full inhibition at 30-33° C. (functional CRISPRi complex) and full induction at 37° C. (non-functional CRISPRi complex) could be an attractive alternative to the experiments shown for the currently known wildtype (wt) Mad7d described above. For other organisms and processes, alternative temperature ranges could be attractive and likely be designed by site-directed mutagenesis of Mad7d.
2) Obtaining Mad7d protein variants that allow for immediate de-stabilization and de-repression by changing temperature. The current Mad7d molecule forms a complex with the gRNA and target DNA, which has a much higher temperature stability once it is established. Screening for Mad7d variants which can be de-stabilized even when already in complex with the gRNA and DNA target would be very valuable. Such Mad7d variants would e.g. enable a temperature inducible system to be set-up, where cell biomass can build up during cultivation at a temperature where the CRISPRi inhibits expression of a gene and then induce expression by increasing temperature to a level where the CRISPRi complex is de-stabilized.

### Example 5. Chromosomal Integration of Gfp and gDNA(Gfp) in B. subtilis

A DNA fragment was constructed for insertion at the B. subtilis amyE locus, where the gfp gene encoding the green fluorescent protein GFP is expressed from the amyL variant promoter P4199, which is earlier described in WO1993010249. Furthermore, a gDNA(gfp) (SEQ ID NO: 11) is expressed from the PamyQ consensus promoter (PamyQsc) described in U.S. Pat. No. 6,255,076. The gDNA(gfp) expresses the gRNA(gfp) with a spacer sequence directing the Mad7d complex to a coding target sequence in the gfp gene. An illustration of the CRISPRi complex binding to the gfp gene is shown in FIG. 14a.

A spectinomycin resistance marker was also included to select for integration. The DNA for integration was ordered as synthetic DNA (GeneArt— ThermoFisher Scientific) and cloned into integration vectors as described in the Material and Methods section. The final map of the amyE locus is shown in FIG. 9. The nucleotide sequence of the locus can be found in SEQ ID NO: 9.

The condition for the PCR amplifications was as follows: The respective DNA fragments were amplified by PCR using the Phusion Hot Start DNA Polymerase system (Thermo Scientific). The PCR amplification reaction mixture contained 1 ul (approx. 0.1 ug) of template DNA, 2 ul of sense primer (20 pmol/ul), 2 ul of anti-sense primer (20 pmol/ul), 10 ul of 5×PCR buffer with 7.5 mM MgCl₂, 8 ul of dNTP mix (1.25 mM each), 37 ul water, and 0.5 ul (2 U/ul) DNA polymerase mix. A thermocycler was used to amplify the fragment. The PCR products were purified from a 1.2% agarose gel with 1×TBE buffer using the Qiagen QIAquick Gel Extraction Kit (Qiagen, Inc., Valencia, Calif.) according to the manufacturer's instructions.

The PCR products were used in a subsequent PCR reaction to create a single fragment by splice overlapping PCR (SOE) using the Phusion Hot Start DNA Polymerase system (Thermo Scientific) as follows. The PCR amplification reaction mixture contained 50 ng of each of the two gel purified PCR products and the synthetic fragment and a thermocycler was used to assemble the DNA for integration. The resulting SOE product was used directly for transformation to B. subtilis host PP2307 to establish the integration by selecting for spectinomycin.

The final construct has the gfp gene expressed from the P4199 promoter and the gDNA(gfp) expressed from the PamyQsc promoter on the chromosome (amyE locus). The strain was named PP5625 (FIG. 9).

### Example 6. Chromosomal Integration of mad7d in B. subtilis

An expression cassette was inserted at the pel locus in which the mad7d gene encoding the Mad7d protein is expressed from the P4199* promoter, which is a variant of the P4199 promoter (WO1993010249) that has a single base change from G to A. A chloramphenicol resistance marker was also included to select for correct integration. The DNA for integration was ordered as synthetic DNA (GeneArt— ThermoFisher Scientific) and cloned into integration vectors as described in the Material and Methods section. The final map of the pel locus is shown in FIG. 10. The nucleotide sequence of the locus can be found in SEQ ID NO: 10.

The PCR products were made as described in example 1 and used in a subsequent PCR reaction to create a single fragment by splice overlapping PCR (SOE) using the Phusion Hot Start DNA Polymerase system (Thermo Scientific) as follows. The PCR amplification reaction mixture contained 50 ng of each of the two gel purified PCR products and the synthetic fragment and a thermocycler was used to assemble and amplify the DNA for integration. The resulting SOE product was used directly for transformation to B. subtilis PP5625. By selection for chloramphenicol resistance the DNA fragment holding the mad7d gene and the cat gene was inserted at the pel locus (FIG. 10).

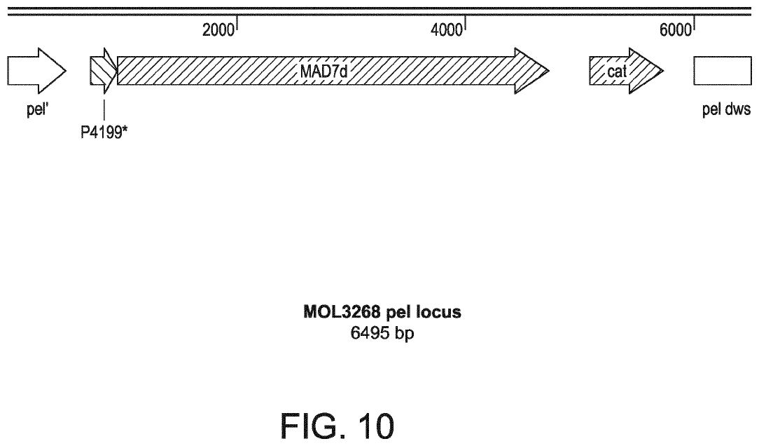

This strain was named MOL3268 and expresses both the Mad7d protein, the GFP protein, and the gRNA(gfp). In FIG. 11 is illustrated how the GFP expression is blocked by CRISPR inhibition inside the gfp gene. The strain MOL3268 is colorless at temperatures below 42° C. because of the CRISPRi complex (Mad7d-gRNA) inhibition of GFP expression.

An illustration of the CRISPRi complex binding to the gfp gene is shown in FIG. 14a.

### Example 7. Identification of Amino Acid Alterations that Will Affect the Thermostability of the Mad7-gRNA-DNA Complex

A protein structure model of Mad7 (SEQ ID NO: 2) from E. rectale (refseq WP 055225123.1) was generated based on the structure of the Cpf1 endonuclease (PDB:SMGA), while the guide RNA (gRNA) and the DNA fragment from the Cas12 endonuclease structure (PDB:SNFV) were adopted into the new structure model, to obtain a complex of endonuclease, gRNA and DNA fragment. This 3D structure complex was analysed in the computer using PyMOL™ Molecular Graphics System (Schrodinger, LLC.) to identify amino acid of key importance for the thermostability of this complex. Changes in these amino acids will lead to destabilizing domain-domain interactions, ion-sites, cysteine bridges, flexible regions, e.g., with high b-factor, salt-bridges, hydrogen bonds and hydrophobic areas in Mad7. Other amino acid changes will destabilize the interactions with the gRNA and the interactions with the DNA.

The changes could either be introduced individually or included as options in one or more libraries, when applying a selective screening system. Mad7 is a large molecule consisting of 1263 amino acids and therefore sufficient destabilization for some biological systems might only be possible by introducing several substitutions simultaneously.

Amino acid alterations within different concepts can be found in Tables 1-3.

TABLE 1
Amino acid residues of importance for Mad7 protein-
protein interactions (protein stability).

| Position | Suggested alterations (substitutions) |
|---|---|
| F34 | S, N, A, L |
| Y61 | S, A, G, L |
| L70 | S, N, A, G |
| K112 | N, S, A, L |
| D130 | S, A, G, L |
| I131 | S, A, G, F |
| L132 | S, N, P, G |
| E134 | S, A, G, L |
| I188 | N, S, A, G |
| G220 | D, N, S, A, G |
| I244 | A, Y, H, G |
| T245 | A, Y, Q, G |
| L287 | A, N, S, G |
| E448 | S, A, G, L |
| A451 | D, N, S, K, G |
| K455 | N, S, A, L |
| E929 | N, S, A, L, G |
| F471 | S, N, A, G |
| V344 | S, N, L, G |
| I518 | S, N, A, Y |
| L520 | S, N, A, Y |
| F522 | Y, S, A, G, L |
| N521 | S, N, L, G |
| W531 | F, S, A, G, L |
| I554 | S, A, G, L |
| F747 | S, A, G, L |
| W682 | S, A, G, L |
| D681 | H, S, A, G, L |
| N732 | S, A, G, L |
| K734 | N, S, L, G |
| L738 | S, A, G, |
| R878 | K, N, S, A, L, H, Q |
| E897 | S, A, G, L |
| Q898 | S, A, G, L |
| F901 | S, A, G, L |
| I930 | S, A, G, L |
| K932 | Q, N, S, A |
| N1004 | S, A, G, L |
| Y1011 | S, A, G, L |
| F1031 | Y, S, A, G, L |
| D1042 | S, A, G, L |
| R51 | K, Q, S, A, G, L |
| L54 | S, A, G, Y |
| I57 | S, A, G, Y |
| M58 | S, A, G, L |
| D60 | S, A, G, L |
| F82 | S, A, G, L, Y |
| K90 | Q, N, S, A |
| K150 | S, A, G, L |
| I154 | S, A, G, F |
| D166 | N, S, A, L |
| F164 | S, A, G, Y, L |
| E194 | N, S, L, G |
| F197 | S, A, G, L |
| Y507 | F, L, S, A, G |
| P297 | L, S, A, G, Q |
| P375 | L, S, A, G, Q |
| P525 | L, S, A, G, Q |
| P574 | L, S, A, G, Q |
| P586 | L, S, A, G, Q |
| P588 | L, S, A, G, Q |
| L713 | S, A, G, N |
| L715 | S, A, G, N |
| L740 | S, A, G, N |
| P756 | L, S, A, G, Q |
| K853 | R, Q, A, P |
| R860 | K, A, Q |
| I865 | A, F, S, N |
| I876 | S, A, Y, N |
| A877 | S, H, G, N, L |
| P1017 | L, S, A, G, Q |
| P1043 | L, S, A, G, Q |
| P1196 | L, S, A, G, Q |
| P1209 | L, S, A, G, Q |
| C174 | S, A, G |
| C185 | S, A, G |
| C288 | S, A, G |
| C1027 | S, A, G |
| C1029 | S, A, G |
| F65 | Y, L, S, A, G |
| K1197 | Q, N, S, A |
| D1200 | E, N, S, L, A |
| K1210 | N, S, A, L |
| A1212 | N, S, G, H, L |
| D1213 | E, N, S, L, G |
| W1101 | F, L, S, H, G |

TABLE 2
Some amino acid residues of importance for Mad7 protein-
gRNA interactions.

| Position | Suggested alterations (substitutions) |
|---|---|
| R833 | K, Q |
| Y834 | F, Q, K, H, N |
| Y836 | F, Q, K, H, N |
| N707 | S, A, G, L |
| D708 | S, A, G, L |
| F709 | Y, Q, K, H, N |
| Y723 | F, Q, K, H, N |

TABLE 3
Some amino acid residues of importance for Mad7
protein-DNA interactions.

| Position | Suggested alterations (substitutions) |
|---|---|
| R1118 | K, H, N |
| F1163 | Y, Q, K, H, N |
| R1167 | K, H, N |
| D1127 | N, S, A |
| T1128 | S, V, A |
| Y294 | F, Q, K, H, N |
| W927 | F, Q, K, H, N |
| K535 | R, H, Q, N |
| K590 | R, H, Q, N |
| K594 | R, H, Q, N |
| K649 | R, H, Q, N |
| K165 | R, H, Q, N |
| R159 | K, H, Q, N |

Examples of combinations of destabilizing substitutions, located within short distance. These can further be combined to destabilize two or more regions of the Mad7-gRNA-DNA complex.

I57S+M58S

F65A+L70S

E194N+F197S

K455N+A451N

K455N+A451N+E448S

L520A+N521A

L520A+N521A+P525G

L520A+F522Y

P525G+W531A

P574Q+P756G

P586G+P588S

D681H+W682A

N707A+K709N+L713A+L715A

N732S+K734N+L738A

L740A+L747S

I876A+A877H+R878S

I876Y+A877L+R878Q

E897S+Q898A+-F901A

E929S+1930S

E929S+I930S+K932N

N1004A+Y1011A

N1004K+Y1011H

C1029A+F1031S

P1196G+K1197N+D1200S

### Example 8. Library of Temperature-Sensitive mad7d Variants in B. subtilis

A library of expression cassettes identical to the one described in Example 6 was inserted at the pel locus as described. Different site-directed variants of the Mad7d protein were constructed based on the proposed modifications on the Mad7 structure further described in Example 7. The site-directed variants were introduced by SOE-PCR and cloned as DNA fragments with flanking DNA identity to the pel locus in B. subtilis as earlier described in WO2006042548. The library of different variants was introduced by transformation into competent PP5625 as described in Example 6. The final map of the pel locus is as already shown in FIG. 10 with the only difference being the different amino acid changes in Mad7d. The library of transformants were screened for green fluorescence at 42° C. to identify temperature sensitive variants within a physiological compatible and relevant temperature interval for Bacillus organisms. Strains with full de-repression at 30° C. were subsequently de-selected. The identified site-directed amino acid substitutions in Mad7d is described in Table 4.

TABLE 4
Amino acid substitutions in temperature sensitive Mad7d variants

| B. subtilis | tsMad7 | |
|---|---|---|
| strain | clones | Amino acid alterations |
| BKQ3716 | TS1 | W531A + P1043L |
| BKQ3717 | TS2 | L699P |
| BKQ3718 | TS3 | N732S + K734N + L738A + D1213N |
| BKQ3775 | TS4 | L70S + D708Y |
| BKQ3776 | TS5 | L132P |
| BKQ3777 | TS6 | C1029A + F1031S |
| BKQ3778 | TS7 | N732S + K734N + L738A |
| BKQ3801 | TS8 | L520A + F522Y + E897S + Q898A + F901A |
| BKQ3803 | TS9 | I57S + M58S + N732S + K734N + L738A |
| BKQ3805 | TS10 | N732S + K734N + L738A + E897S + |
| | | Q898A + F901A |
| BKQ3809 | TS11 | G220D + N732S + K734N + L738A |

### Example 9. Screening of the Mad7d Library by GFP Fluorescence in B. subtilis

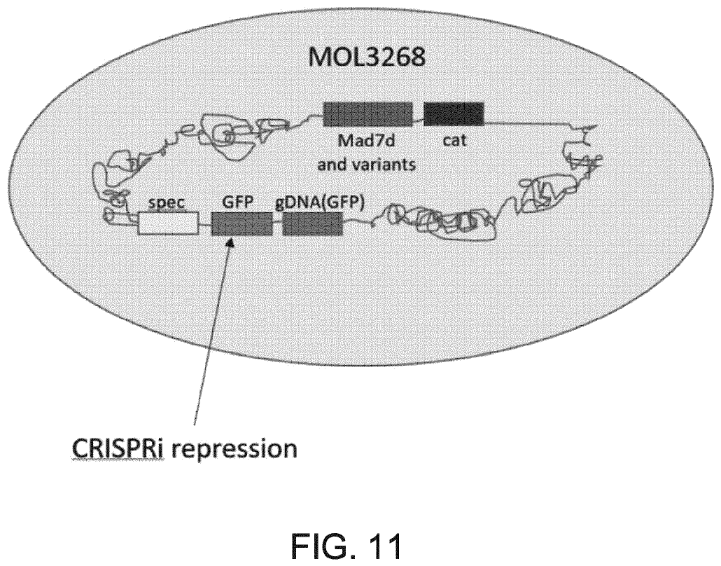

The library of different site-directed Mad7d variants listed in Table 4 were tested for green fluorescence at different temperatures. FIG. 12 shows an example of the plates and their GFP fluorescence. A table listing the qualitative data of the different variants can be seen in Table 5, where the level of fluorescence is scored from zero to four. The zero score corresponds to no fluorescence and the score of four is highest fluorescence.

TABLE 5
Qualitative GFP screening of tsMad7d variants.

| | tsMad7d | | | | |
|---|---|---|---|---|---|
| B. subtilis strain | variant | 30° C. | 34° C. | 37° C. | 42° C. |
| JA1343 (A) | No GFP/ | 0 | 0 | 0 | 0 |
| | No | | | | |
| | Mad7d | | | | |
| PP5625 (B) | No | 4 | 4 | 4 | 4 |
| | Mad7d | | | | |
| MOL3268 (C) | Wt | 0 | 0 | 0 | 0 |
| | Mad7d | | | | |
| BKQ3716 | TS1 | 2 | 2 | 4 | 4 |
| BKQ3717 | TS2 | 2 | 3 | 4 | 4 |
| BKQ3718 | TS3 | 2 | 3 | 3 | 4 |
| BKQ3775 | TS4 | 1 | 2 | 4 | 4 |
| BKQ3776 | TS5 | 0 | 0 | 0 | 1 |
| BKQ3777 | TS6 | 0 | 0 | 1 | 2 |
| BKQ3778 | TS7 | 0 | 1 | 3 | 4 |
| BKQ3801 | TS8 | 0 | 0 | 2 | 4 |
| BKQ3803 | TS9 | 0 | 1 | 3 | 4 |
| BKQ3805 | TS10 | 1 | 2 | 4 | 4 |
| BKQ3809 | TS11 | 0 | 0 | 2 | 4 |

As a negative control the strain JA1343 without GFP and Mad7d show a score of 0 at all temperatures. As a positive control the strain PP5625 show full fluorescence on all temperatures with a score of four. The MOL3268 strain with the wt Mad7d protein show full repression of GFP fluorescence up to 42° C. Strains with the five Mad7d variants BKQ3716(TS1), BKQ3717(TS2), BKQ3718(TS3), BKQ3775(TS4) and BKQ3805(TS10) show slight de-repression already at 30° C. Strains with the two Mad7d variants BKQ3778(TS7), BKQ3803(TS9) start to show de-repression at 34° C., and the two strains with variants BKQ3777(TS6) and BKQ3809(TS11) start to show de-repression at 37° C. Finally, variant BKQ3776(TS5) starts to show slight de-repression at 42° C.

All the Mad7d variants in the table show a pattern of de-repression as a response to temperature, which is very different from the original Mad7d sequence. This will allow a specific design of Mad7d that matches the organism and process for which the inhibition and induction is going to take place in presence of the temperature sensitive Mad7d variant and a functional gRNA towards the target DNA.

### Example 10. Testing the Temperature Sensitive Mad7d Variants in Liquid Cultures

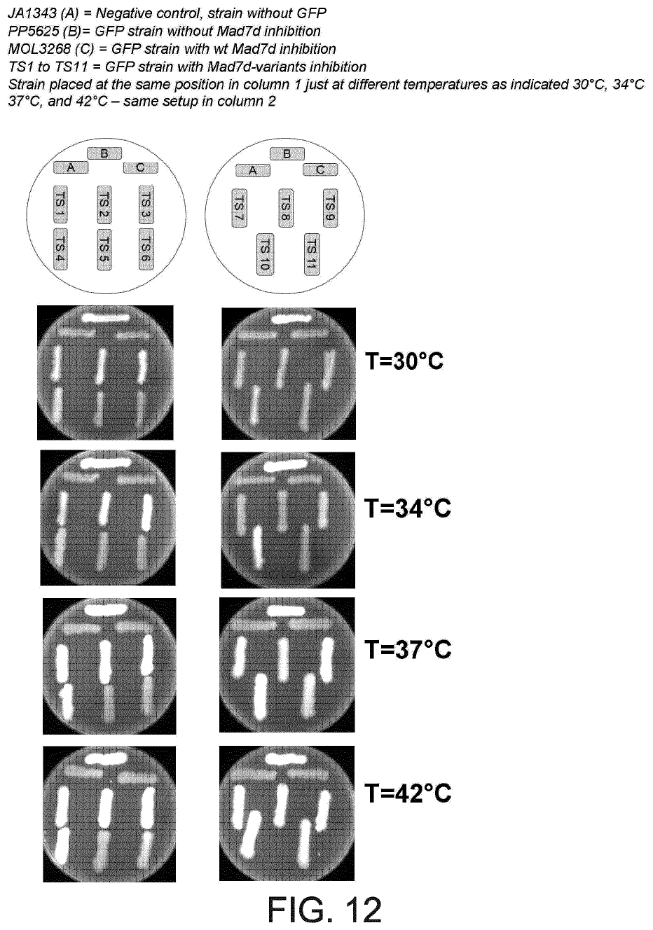

The B. subtilis strains screened for Mad7d variants in Example 9 were also tested in liquid cultures to determine if a response to temperature can be observed as demonstrated on solid media on plates. Two control strains PP5625 and MOL3268 and the individual 11 variant clones were first inoculated in fresh TY media and incubated at 30° C., 34° C., and 37° C. for 18 hours. The positive control strain PP5625 show fluorescence at all temperatures as expected. This strain has no Mad7d gene integrated at the chromosome and cannot form a CRISPRi complex to silence the gfp gene. Consequently, the strain expresses GFP regardless of the growth and temperature. The MOL3268 strain with the wt mad7d gene can form the CRISPRi complex and inhibit expression of the GFP fluorescent protein at all temperatures (FIG. 13). This is in good agreement with the results from Example 9 where the same two strains PP5625 and MOL3268 were tested on solid agar medium. The conclusions from the results on the MOL3268 strain is that the wt Mad7d protein can form a stable CRISPRi complex at temperatures up to at least 37° C. in liquid medium. The eleven Mad7d variants grown in liquid medium also show a reasonable comparable pattern to what was observed on agar plates. The most temperature sensitive variant on plates is also de-repressed in this experiment and is already showing elevated fluorescence at 30° C., such as the variants in BKQ3718 and BKQ3805. Other variants such as in BKQ3716, BKQ3717, BKQ3775, BKQ3803 and BKQ3809 show slight de-repression at 34° C., whereas variants in BKQ3718 and BKQ3805 show moderate de-repression at 34° C. At 37° C. variants in BKQ3776, BKQ3777, BKQ3778 and BKQ3801 show slight de-repression, whereas for the remaining variants the de-repression is elevated at 37° C., compared to 34° C.

The conclusion of this experiment is that the eleven site-directed Mad7d variants all show stability properties that are very different from the original Mad7d protein. All variants show to a varying degree an increased temperature sensitivity both on solid medium and in fluent cultures.

This finding also shows that it is possible to engineer the Mad7d protein in a way that changes the temperature stability of the Mad7d-gRNA inhibitor complex. The changed temperature range can be employed to control the availability of Mad7d-gRNA complex by a switch in temperature which suits the host organism of choice and the physiological conditions of the preferred growth parameters.

### Example 11. Construction of B. licheniformis Strains with Temperature Sensitive Variants TS5, TS6, and TS7

A subset of site-directed variants of Mad7d described in example 9 and 10 for B. subtilis (TS5, TS6, and TS7) were transferred to B. licheniformis strain PP5007 to study the inhibition profile in this organism. The three different variants of mad7d and a gDNA(P4199) targeting the P4199 promoter were each cloned by Prolonged Overlap Extension PCR (POE-PCR) on a plasmid and transformed to B. subtilis BKQ2527. The resulting plasmids were each introduced to B. licheniformis PP5007 by conjugation. Chromosomal insertion of the expression cassette consisting of the mad7d gene expressed from the forD promoter, the gDNA(P4199) transcribed from the PamyQsc promoter and the cat gene conferring chloramphenicol resistance was obtained by homologous recombination, resulting in chromosomal structure as illustrated in FIG. 6. The gDNA(P4199) is transcribed into gRNA(P4199), which directs Mad7d to the P4199 promoter and inhibits transcription. All four amyL gene copies are expressed from the P4199 promoter and the Mad7d-gRNA complex can potentially bind and inhibit alpha-amylase expression through strong interaction to the P4199 target, as illustrated in FIG. 14b. All three strains are identical to BKQ3934 described in example 3 except for the amino acid substitutions in Mad7d described in Table 6.

TABLE 6
B. licheniformis strains containing temperature sensitive
mad7d variants, gDNA(P4199), and four copies of an
amylase expressed from the P4199 promoter.

| B. licheniformis | Mad7d | |
|---|---|---|
| strain | variant | Amino acid substitutions |
| BKQ3934 | Wt Mad7d | No aa substitutions |
| BKQ3913 | TS6 | C1029A F1031S |
| BKQ3917 | TS7 | N732S K734N L738A |
| BKQ3948 | TS5 | L132P |

### Example 12. Alpha-Amylase Expression in B. licheniformis BKQ3913, and BKQ3917 in Fed-Batch Fermentation—Temperature Shift

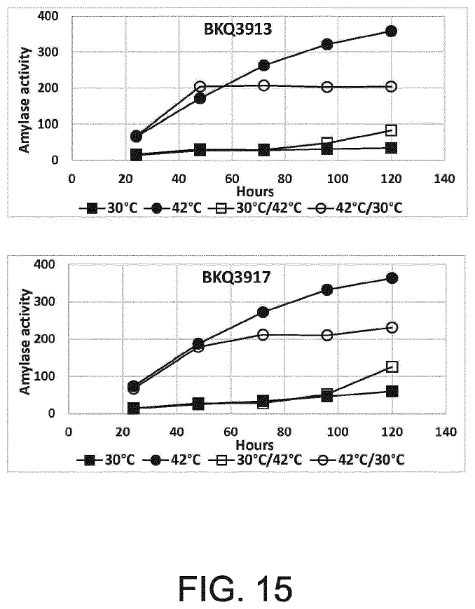

Two of the B. licheniformis strains described in Example 11 were tested with respect to alphaamylase productivity at different temperatures in fed-batch cultivations as described above. The strains were grown for two days either at 30° C. or 42° C. Then the temperatures were shifted to 42° C. and 30° C., respectively, over a time interval of 10 hours to observe the effect on amylase expression from the P4199 promoter. Culture samples were taken each day to measure the activity of amylase. The results are presented in FIG. 15, which show a clear effect on the temperature shifts. Full amylase productivity was observed when the strains were grown at 42° C. for all five days (FIG. 15), showing de-repression of the Mad7d inhibition complex for both variants. When the temperature was lowered to 30° C. after two days, the productivity of amylase was clearly reduced, indicating a repression of transcription from the P4199 promoter. When the strains were grown at 30° C. for the first two days, the presence of the Mad7d variants with the gRNA(P4199) resulted in inhibition of amylase productivity, although BKQ3917 expressing Mad7d variant TS7, was found to be slightly leaky. When the temperature was raised to 42° C., the productivity of amylase started to increase, with BKQ3917 (TS7)>BKQ3913 (TS6). These data show that the inhibition of transcription from the P4199 promoter is de-repressed when the temperature is raised. This is in good agreement with what is observed on plates with the identical Mad7d variants in B. subtilis. The finding that the increase in amylase expression only appears to initiate 24 hours after temperature shift may be related to temperature shock response as was described for PP5007 (no Mad7d) in Example 4. In conclusion, the cultivated B. licheniformis strains confirmed the temperature sensitivity of the used Mad7d variants within an applicable temperature interval to be used for B. licheniformis.

### Example 13. Alpha-Amylase Expression Profile of the Mad7d Variant TS6 in B. Licheniformis BKQ3913

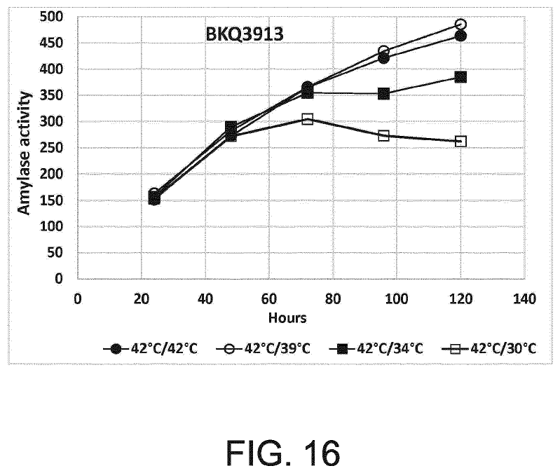

The B. licheniformis strain BKQ3913 was further tested with respect to alpha-amylase productivity and response to a temperature shift in fed-batch cultivation. Full amylase productivity was observed when the strain was grown at 42° C. for the first two days in four separate fermentations (FIG. 16). The temperature was then shifted down to either 30° C., 34° C. and 39° C. in each of the three fermentations, whereas the forth fermentation was kept at 42° C. In FIG. 16 is shown that the downshift to 30° C. efficiently repress the expression of the amylase, while the downshift to 34° C. also represses the expression of amylase though not as efficient. The downshift to 39° C. enables full expression, which shows that this temperature is sufficient to de-stabilize the present Mad7d variant to be unfunctional for silencing of the P4199 promoter.

### Example 14. Alpha-Amylase Expression in B. licheniformis Strains Containing TS5, TS6 and TS7 Cultivated in Fluent Cultures at Different Temperatures

Three B. licheniformis strains described in Example 11 were also tested in liquid cultures to determine if a response to temperature can be observed as demonstrated in B. licheniformis strains expressing amylase gene amyL from P4199 promoters. The B. licheniformis strains containing the temperature sensitive Mad7d variants TS7, TS6, and TS5, respectively, and two control strains PP5007 (no Mad7D) and BKQ3934 (wt Mad7d) were first inoculated in fresh TY media and incubated for 18 hours at 30° C. Next day, the cultures were used to inoculate 20 pl of culture in four 10 ml TY medium each. The cultures were incubated for 20 hours at 30° C., 34° C., 37° C., and 42° C. After incubation, samples were taken to measure amylase activity as described previously.

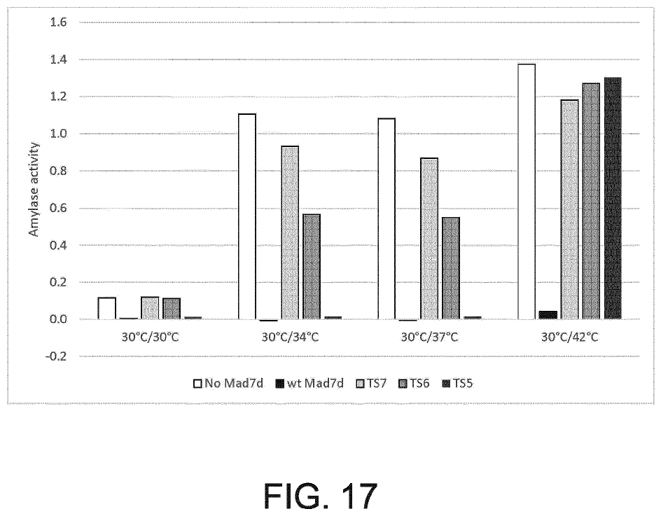

The control strain PP5007 with no Mad7d showed good amylase expression at 34° C., 37° C., and 42° C. As this strain has no Mad7d gene integrated at the chromosome it cannot form an inhibitory CRISPRi complex to silence the P4199 promoters. Consequently, the strain expresses amylase regardless of the growth and temperature. On contrary, the presence of wt Mad7d in BKQ3934 resulted in almost complete silencing of gene expression at all tested temperatures. In this strain a very effective CRISPRi complex can form and inhibit expression from the P4199 promoters at all temperatures (FIG. 17).

The three tested B. licheniformis strains with different Mad7d variants showed varying degree of de-repression of the P4199 promoters. Of the tested variants, the degree of temperature sensitive variants ranged as TS7>TS6>TS5, with TS7 and T6 showing moderate de-repression already at 34° C., whereas TS5 only showed full de-repression at 42° C.

The conclusion of this experiment is that the tested Mad7d variants also in B. licheniformis show stability properties that are very different from the original Mad7d protein. The results further support that different Mad7d variants can be employed to control the availability of Mad7d-gRNA complex by a switch in temperature, which suits the host organism of choice and the physiological conditions of the preferred growth parameters.

### Preferred Embodiments

1) A temperature-sensitive variant of an RNA-guided endonuclease, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 2, and wherein the variant comprises at least one alteration, preferably a substitution, deletion or insertion, of an amino acid important for stability of the RNA-guided endonuclease or for stability of a complex formed between the RNA-guided endonuclease, one or more guide-RNA (gRNA), and/or one or more DNA target sequence.
2) The variant according to the first embodiment, said variant being a nuclease-null variant; preferably said variant comprises an alteration of an amino acid corresponding to position 877 of SEQ ID NO: 2; more preferably said variant comprises a substitution of aspartic acid for alanine, D877A.
3) The variant according to any of embodiments 1-2, wherein the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
4) The variant according to any of embodiments 1-3, wherein the at least one alteration is in a position important for stability of the RNA-guided endonuclease; preferably the at least one alteration is in a positions corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 709, 713, 715, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
5) The variant according to any of embodiments 1-3, wherein the at least one alteration is in a position important for stability of a complex formed between the RNA-guided endonuclease and one or more gRNA; preferably the at least one alteration is in a position corresponding to a position selected from the group consisting of 707, 708, 709, 723, 833, 834, and 836 of SEQ ID NO: 2.
6) The variant according to any of embodiments 1-3 wherein the at least one alteration is in a position important for stability of a complex formed between the RNA-guided endonuclease and one or more DNA target sequence; preferably the at least one alteration is in a positions corresponding to a position selected from the group consisting of 159, 165, 294, 535, 590, 594, 649, 927, 1118, 1127, 1128, 1163, and 1167 of SEQ ID NO: 2.
7) The variant according to any of embodiments 1-3, wherein the at least one alteration is in a position important for stability of the RNA-guided endonuclease and/or for stability of a complex formed between the RNA-guided endonuclease and one or more gRNA; preferably the at least one alteration is in a position corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 707, 708, 709, 713, 715, 723, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
8) The variant according to any of embodiments 1-3, wherein the at least one alteration is in a position important for stability of the RNA-guided endonuclease and/or for stability of a complex formed between the RNA-guided endonuclease and one or more DNA target sequence; preferably the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 709, 713, 715, 732, 734, 738, 740, 747, 756, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
9) The variant according to any of embodiments 1-3, wherein the at least one alteration is in a position important for stability of a complex formed between the RNA-guided endonuclease and one or more gRNA and/or for stability of a complex formed between the RNA-guided endonuclease and one or more DNA target sequence; preferably the at least one alteration is in a position corresponding to a position selected from the group consisting of 159, 165, 294, 535, 590, 594, 649, 707, 708, 709, 723, 833, 834, 836, 927, 1118, 1127, 1128, 1163, and 1167 of SEQ ID NO: 2.
10) The variant according to any of embodiments 1-3, wherein the at least one alteration is in a position corresponding to a position selected from the group consisting of 70, 57, 58, 132, 220, 520, 522, 531, 669, 708, 732, 734, 738, 897, 898, 901, 1029, 1031, 1043, 1094, and 1213 of SEQ ID NO: 2.
11) The variant according to any of embodiments 1-3, wherein the at least one alteration is in a position corresponding to a position selected from the group consisting of 531 and 1034; 699 and 1094; 732, 734, 738, and 1213; 70 and 708; 132; 1029 and 1031; 732, 734, and 738; 520, 522, 897, 898, and 901; 57, 58, 732, 734, and 738; 732, 734, 738, 897, 898, 901; and 220, 732, 734, and 738 of SEQ ID NO: 2.
12) The variant according to any of embodiments 1-3, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L705, L70N, L70A, L70G, F82S, F82A, F82G, F82L, F82Y, K90Q, K90N, K905, K90A, K112N, K1125, K112A, K112L, D1305, D130A, D130G, D130L, 1131S, 1131A, 1131G, 1131F, L132S, L132N, L132P, L132G, E134S, E134A, E134G, E134L, K150S, K150A, K150G, K150L, I154S, I154A, I154G, I154F, F164S, F164A, F164G, F164Y, F164L, D166N, D166S, D166A, D166L, C174S, C174A, C174G, C185S, C185A, C185G, I188N, I188S, I188A, I188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, 0288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
13) The variant according to any of embodiments 1-3, wherein the at least one alteration is a substitution selected from the group consisting of N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, and Y836N.
14) The variant according to any of embodiments 1-3, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
15) The variant according to any of embodiments 1-3, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, 0288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, N707S, N707A, N707G, N707L, D708S, D708A, D708G, D708L, F709Y, F709Q, F709K, F709H, F709N, L713S, L713A, L713G, L713N, L715S, L715A, L715G, L715N, Y723F, Y723Q, Y723K, Y723H, Y723N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, R833K, R833Q, Y834F, Y834Q, Y834K, Y834H, Y834N, Y836F, Y836Q, Y836K, Y836H, Y836N, K853R, K853Q, K853A, K853P, R860K, R860A, R860Q, 1865A, I865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, 1930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
16) The variant according to any of embodiments 1-3, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, R159K, R159H, R159Q, R159N, F164S, F164A, F164G, F164Y, F164L, K165R, K165H, K165Q, K165N, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, 0288A, C288G, Y294F, Y294Q, Y294K, Y294H, Y294N, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, K535R, K535H, K535Q, K535N, 1554S, 1554A, 1554G, 1554L, P574L, P574S, P574A, P574G, P574Q, P586L, P586S, P586A, P586G, P586Q, P588L, P588S, P588A, P588G, P588Q, K590R, K590H, K590Q, K590N, K594R, K594H, K594Q, K594N, K649R, K649H, K649Q, K649N, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K7345, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, W927F, W927Q, W927K, W927H, W927N, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, R1118K, R1118H, R1118N, D1127N, D1127S, D1127A, T1128S, T1128V, T1128A, F1163Y, F1163Q, F1163K, F1163H, F1163N, R1167K, R1167H, R1167N, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
17) The variant according to any of embodiments 1-3, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, Y836N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
18) The variant according to any of embodiments 1-3, wherein the at least one alteration is a substitution selected from the group consisting of 157S, M58S, L70S, L132P, G220D, L520A, F522Y, W531A, L669P, D708Y, N732S, K734N, L738A, E897S, Q898A, F901A, C1029A, F1031S, P1043L, and D1213N.
19) The variant according to any of embodiments 1-3, wherein the at least one alteration is a substitution selected from the group consisting of W531A and P1043L; L699P; N732S, K734N, L738A, and D1213N; L70S and D708Y; L132P; C1029A and F1031S; N732S, K734N, and L738A; L520A, F522Y, E897S, Q898A, and F901A; 157S, M58S, N732S, K734N, and L738A; N732S, K734N, L738A, E897S, Q898A, and F901A; and G220D, N732S, K734N, and L738A.
20) The variant according to any of the preceding embodiments, which has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below; preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
21) The variant according to any of the preceding embodiments, which has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above; preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
22) The variant according to any of the preceding embodiments, wherein the variant has a restrictive temperature that is higher than the permissive temperature.
23) A polynucleotide encoding a temperature-sensitive variant of an RNA-guided endonuclease, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 2, and wherein the variant comprises at least one alteration, preferably a substitution, deletion or insertion, of an amino acid important for stability of the RNA-guided endonuclease or for stability of a complex formed between the RNA-guided endonuclease, one or more guide-RNA (gRNA), and/or one or more DNA target sequence; preferably the polynucleotide has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 1.
24) The polynucleotide according to embodiment 23, wherein the variant is a nuclease-null variant; preferably said variant comprises an alteration of an amino acid corresponding to position 877 of SEQ ID NO: 2; more preferably said variant comprises a substitution of aspartic acid for alanine, D877A.
25) The polynucleotide according to any of embodiments 23-24, wherein the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
26) The polynucleotide according to any of embodiments 23-25, wherein the at least one alteration is in a position important for stability of the RNA-guided endonuclease; preferably the at least one alteration is in a position corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 709, 713, 715, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
27) The polynucleotide according to any of embodiments 23-25, wherein the at least one alteration is in a position important for stability of a complex formed between the RNA-guided endonuclease and one or more gRNA; preferably the at least one alteration is in a position corresponding to a position selected from the group consisting of 707, 708, 709, 723, 833, 834, and 836 of SEQ ID NO: 2.
28) The polynucleotide according to any of embodiments 23-25, wherein the at least one alteration is in a position important for stability of a complex formed between the RNA-guided endonuclease and one or more DNA target sequence; preferably the at least one alteration is in a position corresponding to a position selected from the group consisting of 159, 165, 294, 535, 590, 594, 649, 927, 1118, 1127, 1128, 1163, and 1167 of SEQ ID NO: 2.
29) The polynucleotide according to any of embodiments 23-25, wherein the at least one alteration is in a position important for stability of the RNA-guided endonuclease and/or for stability of a complex formed between the RNA-guided endonuclease and one or more gRNA; preferably the at least one alteration is in a position corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 707, 708, 709, 713, 715, 723, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
30) The polynucleotide according to any of embodiments 23-25, wherein the at least one alteration is in a position important for stability of the RNA-guided endonuclease and/or for stability of a complex formed between the RNA-guided endonuclease and one or more DNA target sequence; preferably the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 709, 713, 715, 732, 734, 738, 740, 747, 756, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
31) The polynucleotide according to any of embodiments 23-25, wherein the at least one alteration is in a position important for stability of a complex formed between the RNA-guided endonuclease and one or more gRNA and/or for stability of a complex formed between the RNA-guided endonuclease and one or more DNA target sequence; preferably the at least one alteration is in a position corresponding to a position selected from the group consisting of 159, 165, 294, 535, 590, 594, 649, 707, 708, 709, 723, 833, 834, 836, 927, 1118, 1127, 1128, 1163, and 1167 of SEQ ID NO: 2.
32) The polynucleotide according to any of embodiments 23-25, wherein the at least one alteration is in a position corresponding to a position selected from the group consisting of 70, 57, 58, 132, 220, 520, 522, 531, 669, 708, 732, 734, 738, 897, 898, 901, 1029, 1031, 1043, 1094, and 1213 of SEQ ID NO: 2.
33) The polynucleotide according any of embodiments 23-25, wherein the at least one alteration is in a position corresponding to a position selected from the group consisting of 531 and 1034; 699 and 1094; 732, 734, 738, and 1213; 70 and 708; 132; 1029 and 1031; 732, 734, and 738; 520, 522, 897, 898, and 901; 57, 58, 732, 734, and 738; 732, 734, 738, 897, 898, 901; and 220, 732, 734, and 738 of SEQ ID NO: 2.
34) The polynucleotide according to any of embodiments 23-25, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L705, L70N, L70A, L70G, F82S, F82A, F82G, F82L, F82Y, K90Q, K90N, K905, K90A, K112N, K1125, K112A, K112L, D1305, D130A, D130G, D130L, 1131S, 1131A, 1131G, 1131F, L132S, L132N, L132P, L132G, E1345, E134A, E134G, E134L, K1505, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E1945, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K4555, K455A, K455L, F471S, F471N, F471A, F471G, Y507F, Y507L, Y5075, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, L7135, L713A, L713G, L713N, L7155, L715A, L715G, L715N, N732S, N732A, N732H, N732L, K734N, K7345, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, I865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
35) The polynucleotide according to any of embodiments 23-25, wherein the at least one alteration is a substitution selected from the group consisting of N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, and Y836N.
36) The polynucleotide according to any of embodiments 23-25, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
37) The polynucleotide according to any of embodiments 23-25, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, I518S, I518N, I518A, I518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, N707S, N707A, N707G, N707L, D708S, D708A, D708G, D708L, F709Y, F709Q, F709K, F709H, F709N, L713S, L713A, L713G, L713N, L715S, L715A, L715G, L715N, Y723F, Y723Q, Y723K, Y723H, Y723N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, R833K, R833Q, Y834F, Y834Q, Y834K, Y834H, Y834N, Y836F, Y836Q, Y836K, Y836H, Y836N, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
38) The polynucleotide according to any of embodiments 23-25, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, R159K, R159H, R159Q, R159N, F164S, F164A, F164G, F164Y, F164L, K165R, K165H, K165Q, K165N, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, I244A, I244Y, I244H, I244G, T245A, T245Y, T245Q, T245G, L287A, L287N, L287S, L287G, C288S, 0288A, C288G, Y294F, Y294Q, Y294K, Y294H, Y294N, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, I518S, I518N, I518A, I518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, K535R, K535H, K535Q, K535N, 1554S, 1554A, 1554G, 1554L, P574L, P574S, P574A, P574G, P574Q, P586L, P586S, P586A, P586G, P586Q, P588L, P588S, P588A, P588G, P588Q, K590R, K590H, K590Q, K590N, K594R, K594H, K594Q, K594N, K649R, K649H, K649Q, K649N, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, W927F, W927Q, W927K, W927H, W927N, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, R1118K, R1118H, R1118N, D1127N, D1127S, D1127A, T1128S, T1128V, T1128A, F1163Y, F1163Q, F1163K, F1163H, F1163N, R1167K, R1167H, R1167N, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
39) The polynucleotide according to any of embodiments 23-25, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, Y836N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
40) The polynucleotide according to any of embodiments 23-25, wherein the at least one alteration is a substitution selected from the group consisting of 157S, M58S, L70S, L132P, G220D, L520A, F522Y, W531A, L669P, D708Y, N732S, K734N, L738A, E897S, Q898A, F901A, C1029A, F1031S, P1043L, and D1213N.
41) The polynucleotide according to any of embodiments 23-25, wherein the at least one alteration include W531A and P1043L; L699P; N732S, K734N, L738A, and D1213N; L70S and D708Y; L132P; C1029A and F1031S; N732S, K734N, and L738A; L520A, F522Y, E897S, Q898A, and F901A; 157S, M58S, N732S, K734N, and L738A; N732S, K734N, L738A, E897S, Q898A, and F901A; and G220D, N732S, K734N, and L738A. 42) The polynucleotide according to any of embodiments 23-41, wherein the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below; preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
43) The polynucleotide according to any embodiments 23-42, wherein the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above; preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
44) The polynucleotide according to any of embodiments 23-43, wherein the variant has a restrictive temperature that is higher than the permissive temperature.
45) A nucleic acid construct comprising a polynucleotide encoding a temperature-sensitive variant of an RNA-guided endonuclease, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 2, and wherein the variant comprises at least one alteration, preferably a substitution, deletion or insertion, of an amino acid important for stability of the RNA-guided endonuclease or for stability of a complex formed between the RNA-guided endonuclease, one or more guide-RNA (gRNA), and/or one or more DNA target sequence; preferably the polynucleotide has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 1.
46) The nucleic acid construct according to embodiment 45, wherein the variant is a nuclease-null variant; preferably said variant comprises an alteration of an amino acid corresponding to position 877 of SEQ ID NO: 2; more preferably said variant comprises a substitution of aspartic acid for alanine, D877A.
47) The nucleic acid construct according to any of embodiments 45-46, wherein the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
48) The nucleic acid construct according to any of embodiments 45-47, wherein the at least one alteration is in a position important for stability of the RNA-guided endonuclease; preferably the at least one alteration is in a position corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 709, 713, 715, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
49) The nucleic acid construct according to any of embodiments 45-47, wherein the at least one alteration is in a position important for stability of a complex formed between the RNA-guided endonuclease and one or more gRNA; preferably the at least one alteration is in a position corresponding to a position selected from the group consisting of 707, 708, 709, 723, 833, 834, and 836 of SEQ ID NO: 2.
50) The nucleic acid construct according to any of embodiments 45-47, wherein the at least one alteration is in a position important for stability of a complex formed between the RNA-guided endonuclease and one or more DNA target sequence; preferably the at least one alteration is in a position corresponding to a position selected from the group consisting of 159, 165, 294, 535, 590, 594, 649, 927, 1118, 1127, 1128, 1163, and 1167 of SEQ ID NO: 2.
51) The nucleic acid construct according to any of embodiments 45-47, wherein the at least one alteration is in a position important for stability of the RNA-guided endonuclease and/or for stability of a complex formed between the RNA-guided endonuclease and one or more gRNA; preferably the at least one alteration is in a position corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 707, 708, 709, 713, 715, 723, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
52) The nucleic acid construct according to any of embodiments 45-47, wherein the at least one alteration is in a position important for stability of the RNA-guided endonuclease and/or for stability of a complex formed between the RNA-guided endonuclease and one or more DNA target sequence; preferably the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 709, 713, 715, 732, 734, 738, 740, 747, 756, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
53) The nucleic acid construct according to any of embodiments 45-47, wherein the at least one alteration is in a position important for stability of a complex formed between the RNA-guided endonuclease and one or more gRNA and/or for stability of a complex formed between the RNA-guided endonuclease and one or more DNA target sequence; preferably the at least one alteration is in a position corresponding to a position selected from the group consisting of 159, 165, 294, 535, 590, 594, 649, 707, 708, 709, 723, 833, 834, 836, 927, 1118, 1127, 1128, 1163, and 1167 of SEQ ID NO: 2.
54) The nucleic acid construct according to any of embodiments 45-47, wherein the at least one alteration is in a position corresponding to a position selected from the group consisting of 70, 57, 58, 132, 220, 520, 522, 531, 669, 708, 732, 734, 738, 897, 898, 901, 1029, 1031, 1043, 1094, and 1213 of SEQ ID NO: 2.
55) The nucleic acid construct according any of embodiments 45-47, wherein the at least one alteration is in a position corresponding to a position selected from the group consisting of 531 and 1034; 699 and 1094; 732, 734, 738, and 1213; 70 and 708; 132; 1029 and 1031; 732, 734, and 738; 520, 522, 897, 898, and 901; 57, 58, 732, 734, and 738; 732, 734, 738, 897, 898, 901; and 220, 732, 734, and 738 of SEQ ID NO: 2.
56) The nucleic acid construct according to any of embodiments 45-47, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, I131S, 1131A, I131G, 1131F, L132S, L132N, L132P, L132G, E134S, E134A, E134G, E134L, K150S, K150A, K150G, K150L, I154S, I154A, I154G, I154F, F164S, F164A, F164G, F164Y, F164L, D166N, D166S, D166A, D166L, C174S, C174A, C174G, C185S, C185A, C185G, I188N, I188S, I188A, I188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, I518S, I518N, I518A, I518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
57) The nucleic acid construct according to any of embodiments 45-47, wherein the at least one alteration is a substitution selected from the group consisting of N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, and Y836N.
58) The nucleic acid construct according to any of embodiments 45-47, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
59) The nucleic acid construct according to any of embodiments 45-47, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, N707S, N707A, N707G, N707L, D708S, D708A, D708G, D708L, F709Y, F709Q, F709K, F709H, F709N, L713S, L713A, L713G, L713N, L715S, L715A, L715G, L715N, Y723F, Y723Q, Y723K, Y723H, Y723N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, R833K, R833Q, Y834F, Y834Q, Y834K, Y834H, Y834N, Y836F, Y836Q, Y836K, Y836H, Y836N, K853R, K853Q, K853A, K853P, R860K, R860A, R860Q, 1865A, I865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
60) The nucleic acid construct according to any of embodiments 45-47, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, R159K, R159H, R159Q, R159N, F164S, F164A, F164G, F164Y, F164L, K165R, K165H, K165Q, K165N, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, 0288A, C288G, Y294F, Y294Q, Y294K, Y294H, Y294N, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, K535R, K535H, K535Q, K535N, 1554S, 1554A, 1554G, 1554L, P574L, P574S, P574A, P574G, P574Q, P586L, P586S, P586A, P586G, P586Q, P588L, P588S, P588A, P588G, P588Q, K590R, K590H, K590Q, K590N, K594R, K594H, K594Q, K594N, K649R, K649H, K649Q, K649N, D681H, D681S, D681A, D681G, D681L, W682S, W682A, VV682G, W682L, L713S, L713A, L713G, L713N, L715S, L715A, L715G, L715N, N732S, N732A, N732H, N732L, K734N, K7345, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, I865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, W927F, W927Q, W927K, W927H, W927N, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, R1118K, R1118H, R1118N, D1127N, D1127S, D1127A, T1128S, T1128V, T1128A, F1163Y, F1163Q, F1163K, F1163H, F1163N, R1167K, R1167H, R1167N, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
61) The nucleic acid construct according to any of embodiments 45-47, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, Y836N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
62) The nucleic acid construct according to any of embodiments 45-47, wherein the at least one alteration is a substitution selected from the group consisting of 157S, M58S, L70S, L132P, G220D, L520A, F522Y, W531A, L669P, D708Y, N732S, K734N, L738A, E897S, Q898A, F901A, C1029A, F1031S, P1043L, and D1213N.
63) The nucleic acid construct according to any of embodiments 45-47, wherein the at least one alteration includes W531A and P1043L; L699P; N732S, K734N, L738A, and D1213N; L70S and D708Y; L132P; C1029A and F1031S; N732S, K734N, and L738A; L520A, F522Y, E897S, Q898A, and F901A; 157S, M58S, N732S, K734N, and L738A; N732S, K734N, L738A, E897S, Q898A, and F901A; and G220D, N732S, K734N, and L738A. 64) The nucleic acid construct according to any of embodiments 45-63, wherein the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below; preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
65) The nucleic acid construct according to any embodiments 45-64, wherein the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above; preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
66) The nucleic acid construct according to any of embodiments 45-65, wherein the variant has a restrictive temperature that is higher than the permissive temperature.
67) An expression vector comprising:

a polynucleotide encoding a temperature-sensitive variant of an RNA-guided endonuclease, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 2, and wherein the variant comprises at least one alteration, preferably a substitution, deletion or insertion, of an amino acid important for stability of the RNA-guided endonuclease or for stability of a complex formed between the RNA-guided endonuclease, one or more guide-RNA (gRNA), and/or one or more DNA target sequence; preferably the polynucleotide has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 1; AND/OR

a nucleic acid construct comprising said polynucleotide.

68) The expression vector according to embodiment 67, wherein the variant is a nuclease-null variant; preferably said variant comprises an alteration of an amino acid corresponding to position 877 of SEQ ID NO: 2; more preferably said variant comprises a substitution of aspartic acid for alanine, D877A.
69) The expression vector according to any of embodiments 67-68, wherein the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
70) The expression vector according to any of embodiments 67-69, wherein the at least one alteration is in a position important for stability of the RNA-guided endonuclease; preferably the at least one alterations is in a positions corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 709, 713, 715, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
71) The expression vector according to any of embodiments 67-70, wherein the at least one alteration is in a position important for stability of a complex formed between the RNA-guided endonuclease and one or more gRNA; preferably the at least one alteration is in a position corresponding to a position selected from the group consisting of 707, 708, 709, 723, 833, 834, and 836 of SEQ ID NO: 2.
72) The expression vector according to any of embodiments 67-71, wherein the at least one alteration is in a position important for stability of a complex formed between the RNA-guided endonuclease and one or more DNA target sequence; preferably the at least one alteration is in a position corresponding to a position selected from the group consisting of 159, 165, 294, 535, 590, 594, 649, 927, 1118, 1127, 1128, 1163, and 1167 of SEQ ID NO: 2.
73) The expression vector according to any of embodiments 67-69, wherein the at least one alteration is in a position important for stability of the RNA-guided endonuclease and/or for stability of a complex formed between the RNA-guided endonuclease and one or more gRNA; preferably the at least one alteration is in a position corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 707, 708, 709, 713, 715, 723, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
74) The expression vector according to any of embodiments 67-69, wherein the at least one alteration is in a position important for stability of the RNA-guided endonuclease and/or for stability of a complex formed between the RNA-guided endonuclease and one or more DNA target sequence; preferably the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 709, 713, 715, 732, 734, 738, 740, 747, 756, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
75) The expression vector according to any of embodiments 67-69, wherein the at least one alteration is in a position important for stability of a complex formed between the RNA-guided endonuclease and one or more gRNA and/or for stability of a complex formed between the RNA-guided endonuclease and one or more DNA target sequence; preferably the at least one alteration is in a position corresponding to a position selected from the group consisting of 159, 165, 294, 535, 590, 594, 649, 707, 708, 709, 723, 833, 834, 836, 927, 1118, 1127, 1128, 1163, and 1167 of SEQ ID NO: 2.
76) The expression vector according to any of embodiments 67-69, wherein the at least one alteration is in a position corresponding to a position selected from the group consisting of 70, 57, 58, 132, 220, 520, 522, 531, 669, 708, 732, 734, 738, 897, 898, 901, 1029, 1031, 1043, 1094, and 1213 of SEQ ID NO: 2.
77) The expression vector according any of embodiments 67-69, wherein the at least one alteration is in a position corresponding to a position selected from the group consisting of 531 and 1034; 699 and 1094; 732, 734, 738, and 1213; 70 and 708; 132; 1029 and 1031; 732, 734, and 738; 520, 522, 897, 898, and 901; 57, 58, 732, 734, and 738; 732, 734, 738, 897, 898, 901; and 220, 732, 734, and 738 of SEQ ID NO: 2.
78) The expression vector according to any of embodiments 67-69, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L705, L70N, L70A, L70G, F82S, F82A, F82G, F82L, F82Y, K90Q, K90N, K905, K90A, K112N, K1125, K112A, K112L, D1305, D130A, D130G, D130L, 1131S, 1131A, 1131G, 1131F, L132S, L132N, L132P, L132G, E1345, E134A, E134G, E134L, K1505, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E1945, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K4555, K455A, K455L, F471S, F471N, F471A, F471G, Y507F, Y507L, Y5075, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
79) The expression vector according to any of embodiments 67-69, wherein the at least one alteration is a substitution selected from the group consisting of N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, and Y836N.
80) The expression vector according to any of embodiments 67-69, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
81) The expression vector according to any of embodiments 67-69, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, I131S, 1131A, I131G, 1131F, L132S, L132N, L132P, L132G, E134S, E134A, E134G, E134L, K150S, K150A, K150G, K150L, I154S, I154A, I154G, I154F, F164S, F164A, F164G, F164Y, F164L, D166N, D166S, D166A, D166L, C174S, C174A, C174G, C185S, C185A, C185G, I188N, I188S, I188A, I188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, I518S, I518N, I518A, I518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, N707S, N707A, N707G, N707L, D708S, D708A, D708G, D708L, F709Y, F709Q, F709K, F709H, F709N, L713S, L713A, L713G, L713N, L715S, L715A, L715G, L715N, Y723F, Y723Q, Y723K, Y723H, Y723N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, R833K, R833Q, Y834F, Y834Q, Y834K, Y834H, Y834N, Y836F, Y836Q, Y836K, Y836H, Y836N, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
82) The expression vector according to any of embodiments 67-69, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, I131S, 1131A, I131G, 1131F, L132S, L132N, L132P, L132G, E134S, E134A, E134G, E134L, K150S, K150A, K150G, K150L, I154S, I154A, I154G, I154F, R159K, R159H, R159Q, R159N, F164S, F164A, F164G, F164Y, F164L, K165R, K165H, K165Q, K165N, D166N, D166S, D166A, D166L, C174S, C174A, C174G, C185S, C185A, C185G, I188N, I188S, I188A, I188G, E194N, E194S, E194L, E194G, F197S, F197A, F197G, F197L, G220D, G220N, G220S, G220A, G220G, I244A, I244Y, I244H, I244G, T245A, T245Y, T245Q, T245G, L287A, L287N, L287S, L287G, C288S, 0288A, C288G, Y294F, Y294Q, Y294K, Y294H, Y294N, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, I518S, I518N, I518A, I518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, K535R, K535H, K535Q, K535N, I554S, I554A, I554G, I554L, P574L, P574S, P574A, P574G, P574Q, P586L, P586S, P586A, P586G, P586Q, P588L, P588S, P588A, P588G, P588Q, K590R, K590H, K590Q, K590N, K594R, K594H, K594Q, K594N, K649R, K649H, K649Q, K649N, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, I865A, I865F, I865S, I865N, I876S, I876A, I876Y, I876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, W927F, W927Q, W927K, W927H, W927N, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, R1118K, R1118H, R1118N, D1127N, D1127S, D1127A, T1128S, T1128V, T1128A, F1163Y, F1163Q, F1163K, F1163H, F1163N, R1167K, R1167H, R1167N, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
83) The expression vector according to any of embodiments 67-69, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, Y836N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
84) The expression vector according to any of embodiments 67-69, wherein the at least one alteration is a substitution selected from the group consisting of 157S, M58S, L70S, L132P, G220D, L520A, F522Y, W531A, L669P, D708Y, N732S, K734N, L738A, E897S, Q898A, F901A, C1029A, F1031S, P1043L, and D1213N.
85) The expression vector according to any of embodiments 67-69, wherein the at least one alteration includes W531A and P1043L; L699P; N732S, K734N, L738A, and D1213N; L70S and D708Y; L132P; C1029A and F1031S; N732S, K734N, and L738A; L520A, F522Y, E897S, Q898A, and F901A; 157S, M58S, N732S, K734N, and L738A; N732S, K734N, L738A, E897S, Q898A, and F901A; and G220D, N732S, K734N, and L738A.
86) The expression vector according to any of embodiments 67-85, wherein the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below; preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
87) The expression vector according to any embodiments 67-86, wherein the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above; preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
88) The expression vector according to any of embodiments 67-87, wherein the variant has a restrictive temperature that is higher than the permissive temperature.
89) A host cell comprising:

a temperature-sensitive variant of an RNA-guided endonuclease, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 2, and wherein the variant comprises at least one alteration, preferably a substitution, deletion or insertion, of an amino acid important for stability of the RNA-guided endonuclease or for stability of a complex formed between the RNA-guided endonuclease, one or more guide-RNA (gRNA), and/or one or more DNA target sequence; AND/OR

a polynucleotide encoding said variant; preferably the polynucleotide has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 1; AND/OR

a nucleic acid construct comprising said polynucleotide; AND/OR

an expression vector comprising said polynucleotide and/or said nucleic acid construct.

90) The host cell according to embodiment 89, wherein the host cell is a eukaryotic or prokaryotic host cell.
91) The host cell according to any of embodiments 89-90, wherein the host cell is a microorganism host cell selected from the group consisting of bacterial, fungal, yeast, and archaeal host cell.
92) The host cell according to any of embodiments 89-91, wherein the host cell is a bacterial host cell selected from the group consisting of Bacillus, Escherichia, Lactobacillus, Lactococcus Streptococcus, and Streptomyces cell; preferably the host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, Bacillus thuringiensis, Escherichia coli, Lactobacillus acidophilus, Lactobacillus amylovorus, Lactobacillus brevis, Lactobacillus (para)casei, Lactobacillus cellobiosus, Lactobacillus crispatus, Lactobacillus curvatus, Lactobacillus delbrueckii subsp. bulgaricus, L. delbrueckii subsp. lactis, Lactobacillus fermentum, Lactobacillus gallinarum, Lactobacillus gasseri, Lacto bacillus helveticus, Lactobacillus johnsonii, Lactobacillus plantarum, Lactobacillus reuteri, Lactobacillus rhamnosus, Lactobacillus salivarius, Lactococcus chungangensis, Lactococcus formosensis, Lactococcus fujiensis, Lactococcus garvieae, Lactococcus lactis, Lactococcus piscium, Lactococcus plantarum, Lactococcus raffinolactis, Lactococcus taiwanensi, Streptococcus equisimilis, Streptococcus pyogenes, Streptococcus uberis, Streptococcus equi subsp. zooepidemicus, Streptomyces achromogenes, Streptomyces avermitilis, Streptomyces coelicolor, Streptomyces griseus, and Streptomyces lividans cell; more preferably the host cell is a Bacillus licheniformis cell or a Bacillus subtilis cell.

93) The host cell according to any of embodiments 89-91, wherein the host cell is a filamentous fungal host cell selected from the group of consisting of Acremonium, Aspergillus, Aureobasidium, Bjerkandera, Ceriporiopsis, Chtysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma Trichoderma longibrachiatum, Trichoderma reesei, and Trichoderma viride cell.

94) The host cell according to any of embodiments 89-92, wherein the host cell is a yeast host cell selected from the group consisting of Candida, Hansenula, Kluyveromyces, Pichia, Saccharomyces, Schizosaccharomyces, and Yarrowia cell; preferably the yeast host cell is selected from the group consisting of Kluyveromyces lactis, Pichia pastoris, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell.
95) The host cell according to any of embodiments 89-94, wherein the variant is a nuclease-null variant; preferably said variant comprises an alteration of an amino acid corresponding to position 877 of SEQ ID NO: 2; more preferably said variant comprises a substitution of aspartic acid for alanine, D877A.
96) The host cell according to any of embodiments 89-95, wherein the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
97) The host cell according to any of embodiments 89-95, wherein the at least one alteration is in a position important for stability of the RNA-guided endonuclease; preferably the at least one alterations is in a positions corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 709, 713, 715, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
98) The host cell according to any of embodiments 89-95, wherein the at least one alteration is in a position important for stability of a complex formed between the RNA-guided endonuclease and one or more gRNA; preferably the at least one alteration is in a position corresponding to a position selected from the group consisting of 707, 708, 709, 723, 833, 834, and 836 of SEQ ID NO: 2.
99) The host cell according to any of embodiments 89-95, wherein the at least one alteration is in a position important for stability of a complex formed between the RNA-guided endonuclease and one or more DNA target sequence; preferably the at least one alteration is in a position corresponding to a position selected from the group consisting of 159, 165, 294, 535, 590, 594, 649, 927, 1118, 1127, 1128, 1163, and 1167 of SEQ ID NO: 2.
100) The host cell according to any of embodiments 89-95, wherein the at least one alteration is in a position important for stability of the RNA-guided endonuclease and/or for stability of a complex formed between the RNA-guided endonuclease and one or more gRNA; preferably the at least one alteration is in a position corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 707, 708, 709, 713, 715, 723, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
101) The host cell according to any of embodiments 89-95, wherein the at least one alteration is in a position important for stability of the RNA-guided endonuclease and/or for stability of a complex formed between the RNA-guided endonuclease and one or more DNA target sequence; preferably the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 709, 713, 715, 732, 734, 738, 740, 747, 756, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
102) The host cell according to any of embodiments 89-95, wherein the at least one alteration is in a position important for stability of a complex formed between the RNA-guided endonuclease and one or more gRNA and/or for stability of a complex formed between the RNA-guided endonuclease and one or more DNA target sequence; preferably the at least one alteration is in a position corresponding to a position selected from the group consisting of 159, 165, 294, 535, 590, 594, 649, 707, 708, 709, 723, 833, 834, 836, 927, 1118, 1127, 1128, 1163, and 1167 of SEQ ID NO: 2.
103) The host cell according to any of embodiments 89-95, wherein the at least one alteration is in a position corresponding to a position selected from the group consisting of 70, 57, 58, 132, 220, 520, 522, 531, 669, 708, 732, 734, 738, 897, 898, 901, 1029, 1031, 1043, 1094, and 1213 of SEQ ID NO: 2.
104) The host cell according any of embodiments 89-95, wherein the at least one alteration is in a position corresponding to a position selected from the group consisting of 531 and 1034; 699 and 1094; 732, 734, 738, and 1213; 70 and 708; 132; 1029 and 1031; 732, 734, and 738; 520, 522, 897, 898, and 901; 57, 58, 732, 734, and 738; 732, 734, 738, 897, 898, 901; and 220, 732, 734, and 738 of SEQ ID NO: 2.
105) The host cell according to any of embodiments 89-95, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L705, L70N, L70A, L70G, F82S, F82A, F82G, F82L, F82Y, K90Q, K90N, K905, K90A, K112N, K1125, K112A, K112L, D1305, D130A, D130G, D130L, 1131S, 1131A, 1131G, 1131F, L132S, L132N, L132P, L132G, E1345, E134A, E134G, E134L, K1505, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E1945, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
106) The host cell according to any of embodiments 89-95, wherein the at least one alteration is a substitution selected from the group consisting of N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, and Y836N.
107) The host cell according to any of embodiments 89-95, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
108) The host cell according to any of embodiments 89-95, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, N707S, N707A, N707G, N707L, D708S, D708A, D708G, D708L, F709Y, F709Q, F709K, F709H, F709N, L713S, L713A, L713G, L713N, L715S, L715A, L715G, L715N, Y723F, Y723Q, Y723K, Y723H, Y723N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, R833K, R833Q, Y834F, Y834Q, Y834K, Y834H, Y834N, Y836F, Y836Q, Y836K, Y836H, Y836N, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
109) The host cell according to any of embodiments 89-95, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, R159K, R159H, R159Q, R159N, F164S, F164A, F164G, F164Y, F164L, K165R, K165H, K165Q, K165N, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, 0288A, C288G, Y294F, Y294Q, Y294K, Y294H, Y294N, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, K535R, K535H, K535Q, K535N, 1554S, 1554A, 1554G, 1554L, P574L, P574S, P574A, P574G, P574Q, P586L, P586S, P586A, P586G, P586Q, P588L, P588S, P588A, P588G, P588Q, K590R, K590H, K590Q, K590N, K594R, K594H, K594Q, K594N, K649R, K649H, K649Q, K649N, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, W927F, W927Q, W927K, W927H, W927N, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, R1118K, R1118H, R1118N, D1127N, D1127S, D1127A, T1128S, T1128V, T1128A, F1163Y, F1163Q, F1163K, F1163H, F1163N, R1167K, R1167H, R1167N, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
110) The host cell according to any of embodiments 89-95, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, Y836N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
111) The host cell according to any of embodiments 89-95, wherein the at least one alteration is a substitution selected from the group consisting of 157S, M58S, L70S, L132P, G220D, L520A, F522Y, W531A, L669P, D708Y, N732S, K734N, L738A, E897S, Q898A, F901A, C1029A, F1031S, P1043L, and D1213N.
112) The host cell according to any of embodiments 89-95, wherein the at least one alteration includes W531A and P1043L; L699P; N732S, K734N, L738A, and D1213N; L70S and D708Y; L132P; C1029A and F1031S; N732S, K734N, and L738A; L520A, F522Y, E897S, Q898A, and F901A; 157S, M58S, N732S, K734N, and L738A; N732S, K734N, L738A, E897S, Q898A, and F901A; and G220D, N732S, K734N, and L738A.
113) The host cell according to any of embodiments 89-95, wherein the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below; preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
114) The host cell according to any embodiments 89-113, wherein the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above; preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
115) The host cell according to any of embodiments 89-114, wherein the variant has a restrictive temperature that is higher than the permissive temperature.
116) A method of inducing expression of one or more DNA target sequence of interest, the method comprising the steps of:
a) providing a host cell comprising:

a polynucleotide encoding a temperature-sensitive variant of a catalytically inactive RNA-guided endonuclease, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 2, and wherein the variant comprises at least one alteration, preferably a substitution, deletion or insertion, of an amino acid important for stability of the RNA-guided endonuclease or for stability of a complex formed between the RNA-guided endonuclease, one or more guide-RNA (gRNA), and/or one or more DNA target sequence; preferably the polynucleotide has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 1; AND/OR

a nucleic acid construct comprising said polynucleotide; AND/OR

an expression vector comprising said polynucleotide and/or said nucleic acid construct;

said host cell further comprising one or more gRNA and one or more DNA target sequence of interest;

b) cultivating the host cell at a permissive temperature of the variant and under conditions conducive for expression of the variant, whereby a complex is formed in the host cell between the variant with the one or more gRNA and the one or more DNA target sequence, and whereby expression of the one or more DNA target sequence is repressed; and subsequently

c) increasing the temperature to a restrictive temperature of the variant and cultivating the host cell, whereby the formed complex dissociates and expression of the one or more DNA target sequence is induced.

117) The method according to embodiment 116, wherein the host cell is a eukaryotic or prokaryotic host cell.
118) The method according to any of embodiments 116-117, wherein the host cell is a microorganism host cell selected from the group consisting of bacterial, fungal, yeast, and archaeal host cell.
119) The method according to any of embodiments 116-118, wherein the host cell is a bacterial host cell selected from the group consisting of Bacillus, Escherichia, Lactobacillus, Lactococcus Streptococcus, and Streptomyces cell; preferably the host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, Bacillus thuringiensis, Escherichia coli, Lactobacillus acidophilus, Lactobacillus amylovorus, Lactobacillus brevis, Lactobacillus (para)casei, Lactobacillus cellobiosus, Lactobacillus crispatus, Lactobacillus curvatus, Lactobacillus delbrueckii subsp. bulgaricus, L. delbrueckii subsp. lactis, Lactobacillus fermentum, Lactobacillus gallinarum, Lactobacillus gasseri, Lacto bacillus helveticus, Lactobacillus johnsonii, Lactobacillus plantarum, Lactobacillus reuteri, Lactobacillus rhamnosus, Lactobacillus salivarius, Lactococcus chungangensis, Lactococcus formosensis, Lactococcus fujiensis, Lactococcus garvieae, Lactococcus lactis, Lactococcus piscium, Lactococcus plantarum, Lactococcus raffinolactis, Lactococcus taiwanensi, Streptococcus equisimilis, Streptococcus pyogenes, Streptococcus uberis, Streptococcus equi subsp. zooepidemicus, Streptomyces achromogenes, Streptomyces avermitilis, Streptomyces coelicolor, Streptomyces griseus, and Streptomyces lividans cell; more preferably the host cell is a Bacillus licheniformis cell or a Bacillus subtilis cell.

120) The method according to any of embodiments 116-118, wherein the host cell is a filamentous fungal host cell selected from the group of consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell.

121) The method according to any of embodiments 116-118, wherein the host cell is a yeast host cell selected from the group consisting of Candida, Hansenula, Kluyveromyces, Pichia, Saccharomyces, Schizosaccharomyces, and Yarrowia cell; preferably the yeast host cell is selected from the group consisting of Kluyveromyces lactis, Pichia pastoris, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell.

122) The method according to any of embodiments 116-121, wherein the variant comprises an alteration of an amino acid corresponding to position 877 of SEQ ID NO: 2; preferably said variant comprises a substitution of aspartic acid for alanine, D877A.
123) The method according to any of embodiments 116-122, wherein the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
124) The method according to any of embodiments 116-122, wherein the at least one alteration is in a position important for stability of the RNA-guided endonuclease; preferably the at least one alterations is in a positions corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 709, 713, 715, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
125) The method according to any of embodiments 116-122, wherein the at least one alteration is in a position important for stability of a complex formed between the RNA-guided endonuclease and one or more gRNA; preferably the at least one alteration is in a position corresponding to a position selected from the group consisting of 707, 708, 709, 723, 833, 834, and 836 of SEQ ID NO: 2.
126) The method according to any of embodiments 116-122, wherein the at least one alteration is in a position important for stability of a complex formed between the RNA-guided endonuclease and one or more DNA target sequence; preferably the at least one alteration is in a position corresponding to a position selected from the group consisting of 159, 165, 294, 535, 590, 594, 649, 927, 1118, 1127, 1128, 1163, and 1167 of SEQ ID NO: 2.
127) The method according to any of embodiments 116-122, wherein the at least one alteration is in a position important for stability of the RNA-guided endonuclease and/or for stability of a complex formed between the RNA-guided endonuclease and one or more gRNA; preferably the at least one alteration is in a position corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 707, 708, 709, 713, 715, 723, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
128) The method according to any of embodiments 116-122, wherein the at least one alteration is in a position important for stability of the RNA-guided endonuclease and/or for stability of a complex formed between the RNA-guided endonuclease and one or more DNA target sequence; preferably the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 709, 713, 715, 732, 734, 738, 740, 747, 756, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
129) The method according to any of embodiments 116-122, wherein the at least one alteration is in a position important for stability of a complex formed between the RNA-guided endonuclease and one or more gRNA and/or for stability of a complex formed between the RNA-guided endonuclease and one or more DNA target sequence; preferably the at least one alteration is in a position corresponding to a position selected from the group consisting of 159, 165, 294, 535, 590, 594, 649, 707, 708, 709, 723, 833, 834, 836, 927, 1118, 1127, 1128, 1163, and 1167 of SEQ ID NO: 2.
130) The method according to any of embodiments 116-122, wherein the at least one alteration is in a position corresponding to a position selected from the group consisting of 70, 57, 58, 132, 220, 520, 522, 531, 669, 708, 732, 734, 738, 897, 898, 901, 1029, 1031, 1043, 1094, and 1213 of SEQ ID NO: 2.
131) The method according any of embodiments 116-122, wherein the at least one alteration is in a position corresponding to a position selected from the group consisting of 531 and 1034; 699 and 1094; 732, 734, 738, and 1213; 70 and 708; 132; 1029 and 1031; 732, 734, and 738; 520, 522, 897, 898, and 901; 57, 58, 732, 734, and 738; 732, 734, 738, 897, 898, 901; and 220, 732, 734, and 738 of SEQ ID NO: 2.
132) The method according to any of embodiments 116-122, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L705, L70N, L70A, L70G, F82S, F82A, F82G, F82L, F82Y, K90Q, K90N, K905, K90A, K112N, K1125, K112A, K112L, D1305, D130A, D130G, D130L, 1131S, 1131A, 1131G, 1131F, L132S, L132N, L132P, L132G, E1345, E134A, E134G, E134L, K1505, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E1945, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K4555, K455A, K455L, F471S, F471N, F471A, F471G, Y507F, Y507L, Y5075, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, L7135, L713A, L713G, L713N, L7155, L715A, L715G, L715N, N732S, N732A, N732H, N732L, K734N, K7345, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, I865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y10115, Y1011A, Y1011G, Y1011L, P1017L, P10175, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
133) The method according to any of embodiments 116-122, wherein the at least one alteration is a substitution selected from the group consisting of N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, and Y836N.
134) The method according to any of embodiments 116-122 wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
135) The method according to any of embodiments 116-122, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, N707S, N707A, N707G, N707L, D708S, D708A, D708G, D708L, F709Y, F709Q, F709K, F709H, F709N, L713S, L713A, L713G, L713N, L715S, L715A, L715G, L715N, Y723F, Y723Q, Y723K, Y723H, Y723N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, R833K, R833Q, Y834F, Y834Q, Y834K, Y834H, Y834N, Y836F, Y836Q, Y836K, Y836H, Y836N, K853R, K853Q, K853A, K853P, R860K, R860A, R860Q, 1865A, 1865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
136) The method according to any of embodiments 116-122, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, R159K, R159H, R159Q, R159N, F164S, F164A, F164G, F164Y, F164L, K165R, K165H, K165Q, K165N, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, 0288A, C288G, Y294F, Y294Q, Y294K, Y294H, Y294N, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, K535R, K535H, K535Q, K535N, I554S, I554A, I554G, I554L, P574L, P574S, P574A, P574G, P574Q, P586L, P586S, P586A, P586G, P586Q, P588L, P588S, P588A, P588G, P588Q, K590R, K590H, K590Q, K590N, K594R, K594H, K594Q, K594N, K649R, K649H, K649Q, K649N, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, I865A, I865F, I865S, I865N, I876S, I876A, I876Y, I876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, W927F, W927Q, W927K, W927H, W927N, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, R1118K, R1118H, R1118N, D1127N, D1127S, D1127A, T1128S, T1128V, T1128A, F1163Y, F1163Q, F1163K, F1163H, F1163N, R1167K, R1167H, R1167N, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
137) The method according to any of embodiments 116-122, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, Y836N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
138) The method according to any of embodiments 116-122, wherein the at least one alteration is a substitution selected from the group consisting of 157S, M58S, L70S, L132P, G220D, L520A, F522Y, W531A, L669P, D708Y, N732S, K734N, L738A, E897S, Q898A, F901A, C1029A, F1031S, P1043L, and D1213N.
139) The method according to any of embodiments 116-122, wherein the at least one alteration includes W531A and P1043L; L699P; N732S, K734N, L738A, and D1213N; L70S and D708Y; L132P; C1029A and F1031S; N732S, K734N, and L738A; L520A, F522Y, E897S, Q898A, and F901A; 157S, M58S, N732S, K734N, and L738A; N732S, K734N, L738A, E897S, Q898A, and F901A; and G220D, N732S, K734N, and L738A.
140) The method according to any of embodiments 116-122, wherein the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below; preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.

141) The method according to any embodiments 116-140, wherein the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above; preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.

142) The method according to any of embodiments 116-141, wherein the variant has a restrictive temperature that is higher than the permissive temperature.
143) The method according to any of embodiments 116-141, said method comprising the additional step of:

d) decreasing the temperature to a permissive temperature of the variant, wherein a complex is formed in the host cell by the variant, the one or more gRNA, and the one or more DNA target sequence, and cultivating the host cell, whereby expression of the one or more DNA target sequence is repressed.

144) A method of repressing one or more DNA target sequence of interest, the method comprising the steps of:
a) providing a host cell comprising:

a polynucleotide encoding a temperature-sensitive variant of a catalytically inactive RNA-guided endonuclease, wherein the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 2, and wherein the variant comprises at least one alteration, preferably a substitution, deletion or insertion, of an amino acid important for stability of the RNA-guided endonuclease or for stability of a complex formed between the RNA-guided endonuclease, one or more guide-RNA (gRNA), and/or one or more DNA target sequence; preferably the polynucleotide has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100%, to SEQ ID NO: 1; AND/OR

a nucleic acid construct comprising said polynucleotide; AND/OR

an expression vector comprising said polynucleotide and/or said nucleic acid construct;

said host cell further comprising one or more gRNA and one or more DNA target sequence of interest;

b) cultivating the host cell at a restrictive temperature of the variant and under conditions conducive for expression of the variant, wherein the one or more DNA target sequence is expressed; and subsequently

c) decreasing the temperature to a permissive temperature of the variant and cultivating the host cell, whereby a complex is formed in the host cell between the variant, the one or more gRNA and the one or more DNA target sequence, and whereby expression of the one or more DNA target sequence is repressed.

145) The method according to embodiment 144, wherein the host cell is a eukaryotic or prokaryotic host cell.
146) The method according to any of embodiments 144-145, wherein the host cell is a microorganism host cell selected from the group consisting of bacterial, fungal, yeast, and archaeal host cell.
147) The method according to any of embodiments 144-146, wherein the host cell is a bacterial host cell selected from the group consisting of Bacillus, Escherichia, Lactobacillus, Lactococcus Streptococcus, and Streptomyces cell; preferably the host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, Bacillus thuringiensis, Escherichia coli, Lactobacillus acidophilus, Lactobacillus amylovorus, Lactobacillus brevis, Lactobacillus (para)casei, Lactobacillus cellobiosus, Lactobacillus crispatus, Lactobacillus curvatus, Lactobacillus delbrueckii subsp. bulgaricus, L. delbrueckii subsp. lactis, Lactobacillus fermentum, Lactobacillus gallinarum, Lactobacillus gasseri, Lacto bacillus helveticus, Lactobacillus johnsonii, Lactobacillus plantarum, Lactobacillus reuteri, Lactobacillus rhamnosus, Lactobacillus salivarius, Lactococcus chungangensis, Lactococcus formosensis, Lactococcus fujiensis, Lactococcus garvieae, Lactococcus lactis, Lactococcus piscium, Lactococcus plantarum, Lactococcus raffinolactis, Lactococcus taiwanensi, Streptococcus equisimilis, Streptococcus pyogenes, Streptococcus uberis, Streptococcus equi subsp. zooepidemicus, Streptomyces achromogenes, Streptomyces avermitilis, Streptomyces coelicolor, Streptomyces griseus, and Streptomyces lividans cell; more preferably the host cell is a Bacillus licheniformis cell or a Bacillus subtilis cell.
148) The method according to any of embodiments 144-146, wherein the host cell is a filamentous fungal host cell selected from the group of consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell.
149) The method according to any of embodiments 144-146, wherein the host cell is a yeast host cell selected from the group consisting of Candida, Hansenula, Kluyveromyces, Pichia, Saccharomyces, Schizosaccharomyces, and Yarrowia cell; preferably the yeast host cell is selected from the group consisting of Kluyveromyces lactis, Pichia pastoris, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell.
150) The method according to any of embodiments 144-149, wherein the variant comprises an alteration of an amino acid corresponding to position 877 of SEQ ID NO: 2; preferably said variant comprises a substitution of aspartic acid for alanine, D877A.
151) The method according to any of embodiments 144-150, wherein the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
152) The method according to any of embodiments 144-151, wherein the at least one alteration is in a position important for stability of the RNA-guided endonuclease; preferably the at least one alterations is in a positions corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 709, 713, 715, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
153) The method according to any of embodiments 144-151, wherein the at least one alteration is in a position important for stability of a complex formed between the RNA-guided endonuclease and one or more gRNA; preferably the at least one alteration is in a position corresponding to a position selected from the group consisting of 707, 708, 709, 723, 833, 834, and 836 of SEQ ID NO: 2.
154) The method according to any of embodiments 144-151, wherein the at least one alteration is in a position important for stability of a complex formed between the RNA-guided endonuclease and one or more DNA target sequence; preferably the at least one alteration is in a position corresponding to a position selected from the group consisting of 159, 165, 294, 535, 590, 594, 649, 927, 1118, 1127, 1128, 1163, and 1167 of SEQ ID NO: 2.
155) The method according to any of embodiments 144-151, wherein the at least one alteration is in a position important for stability of the RNA-guided endonuclease and/or for stability of a complex formed between the RNA-guided endonuclease and one or more gRNA; preferably the at least one alteration is in a position corresponding to a position selected from the group consisting of 34, 51, 54, 57, 58, 60, 61, 65, 70, 82, 90, 112, 130, 131, 132, 134, 150, 154, 164, 166, 174, 185, 188, 194, 197, 220, 244, 245, 287, 288, 297, 344, 375, 448, 451, 455, 471, 507, 518, 520, 521, 522, 525, 531, 554, 571, 574, 586, 588, 681, 682, 707, 708, 709, 713, 715, 723, 732, 734, 738, 740, 747, 756, 853, 860, 865, 876, 878, 897, 898, 901, 929, 930, 932, 1004, 1017, 1011, 1027, 1029, 1031, 1042, 1043, 1101, 1196, 1197, 1200, 1209, 1210, 1212, and 1213 of SEQ ID NO: 2.
156) The method according to any of embodiments 144-151, wherein the at least one alteration is in a position important for stability of the RNA-guided endonuclease and/or for stability of a complex formed between the RNA-guided endonuclease and one or more DNA target sequence; preferably the at least one alteration is in a position corresponding to a position selected from the group consisting of 34, 51, 54, 57, 58, 60, 61, 65, 70, 82, 90, 112, 130, 131, 132, 134, 150, 154, 159, 164, 165, 166, 174, 185, 188, 194, 197, 220, 244, 245, 287, 288, 294, 297, 344, 375, 448, 451, 455, 471, 507, 518, 520, 521, 522, 525, 531, 535, 554, 571, 574, 586, 588, 590, 594, 649, 681, 682, 709, 713, 715, 732, 734, 738, 740, 747, 756, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1167, 1196, 1197, 1200, 1209, 1210, 1212, and 1213 of SEQ ID NO: 2.
157) The method according to any of embodiments 144-151, wherein the at least one alteration is in a position important for stability of a complex formed between the RNA-guided endonuclease and one or more gRNA and/or for stability of a complex formed between the RNA-guided endonuclease and one or more DNA target sequence; preferably the at least one alteration is in a position corresponding to a position selected from the group consisting of 159, 165, 294, 535, 590, 594, 649, 707, 708, 709, 723, 833, 834, 836, 927, 1118, 1127, 1128, 1163, and 1167 of SEQ ID NO: 2.
158) The method according to any of embodiments 144-151, wherein the at least one alteration is in a position corresponding to a position selected from the group consisting of 70, 57, 58, 132, 220, 520, 522, 531, 669, 708, 732, 734, 738, 897, 898, 901, 1029, 1031, 1043, 1094, and 1213 of SEQ ID NO: 2.
159) The method according any of embodiments 144-151, wherein the at least one alteration is in a position corresponding to a position selected from the group consisting of 531 and 1034; 699 and 1094; 732, 734, 738, and 1213; 70 and 708; 132; 1029 and 1031; 732, 734, and 738; 520, 522, 897, 898, and 901; 57, 58, 732, 734, and 738; 732, 734, 738, 897, 898, 901; and 220, 732, 734, and 738 of SEQ ID NO: 2.
160) The method according to any of embodiments 144-151, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, I131S, 1131A, I131G, 1131F, L132S, L132N, L132P, L132G, E134S, E134A, E134G, E134L, K150S, K150A, K150G, K150L, I154S, I154A, I154G, I154F, F164S, F164A, F164G, F164Y, F164L, D166N, D166S, D166A, D166L, C174S, C174A, C174G, C185S, C185A, C185G, I188N, I188S, I188A, I188G, E194N, E194S, E194L, E194G, F197S, F197A, F197G, F197L, G220D, G220N, G220S, G220A, G220G, I244A, I244Y, I244H, I244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, I518S, I518N, I518A, I518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, I554S, I554A, I554G, I554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, I865A, I865F, I865S, I865N, I876S, I876A, I876Y, I876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
161) The method according to any of embodiments 144-151, wherein the at least one alteration is a substitution selected from the group consisting of N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, and Y836N.
162) The method according to any of embodiments 144-151, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
163) The method according to any of embodiments 144-151, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, F164S, F164A, F164G, F164Y, F164L, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, C288A, C288G, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, 1554S, 1554A, 1554G, 1554L, P574L, P574S, P574A, P574G, P574Q, P586L, P586S, P586A, P586G, P586Q, P588L, P588S, P588A, P588G, P588Q, D681H, D681S, D681A, D681G, D681L, W682S, W682A, W682G, W682L, N707S, N707A, N707G, N707L, D708S, D708A, D708G, D708L, F709Y, F709Q, F709K, F709H, F709N, L713S, L713A, L713G, L713N, L715S, L715A, L715G, L715N, Y723F, Y723Q, Y723K, Y723H, Y723N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, R833K, R833Q, Y834F, Y834Q, Y834K, Y834H, Y834N, Y836F, Y836Q, Y836K, Y836H, Y836N, K853R, K853Q, K853A, K853P, R860K, R860A, R860Q, 1865A, I865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
164) The method according to any of embodiments 144-151, wherein the at least one alteration is a substitution selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L70S, L70N, L70A, L70G, F82S, F82A, F82G, F82L, F82Y, K90Q, K90N, K90S, K90A, K112N, K112S, K112A, K112L, D130S, D130A, D130G, D130L, 1131S, 1131A, 1131G, 1131F, L132S, L132N, L132P, L132G, E134S, E134A, E134G, E134L, K150S, K150A, K150G, K150L, 1154S, 1154A, 1154G, 1154F, R159K, R159H, R159Q, R159N, F164S, F164A, F164G, F164Y, F164L, K165R, K165H, K165Q, K165N, D166N, D166S, D166A, D166L, C174S, C174A, C174G, C185S, C185A, C185G, 1188N, 1188S, 1188A, 1188G, E194N, E194S, E194L, E194G, F197S, F197A, F197G, F197L, G220D, G220N, G220S, G220A, G220G, 1244A, 1244Y, 1244H, 1244G, T245A, T245Y, T245Q, T245G, L287A, L287N, L287S, L287G, C288S, 0288A, C288G, Y294F, Y294Q, Y294K, Y294H, Y294N, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, 1518S, 1518N, 1518A, 1518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, K535R, K535H, K535Q, K535N, 1554S, 1554A, 1554G, 1554L, P574L, P574S, P574A, P574G, P574Q, P586L, P586S, P586A, P586G, P586Q, P588L, P588S, P588A, P588G, P588Q, K590R, K590H, K590Q, K590N, K594R, K594H, K594Q, K594N, K649R, K649H, K649Q, K649N, D681H, D681S, D681A, D681G, D681L, W682S, W682A, W682G, W682L, L713S, L713A, L713G, L713N, L715S, L715A, L715G, L715N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, K853R, K853Q, K853A, K853P, R860K, R860A, R860Q, 1865A, I865F, 1865S, 1865N, 1876S, 1876A, 1876Y, 1876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, W927F, W927Q, W927K, W927H, W927N, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H, W1101G, R1118K, R1118H, R1118N, D1127N, D1127S, D1127A, T1128S, T1128V, T1128A, F1163Y, F1163Q, F1163K, F1163H, F1163N, R1167K, R1167H, R1167N, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.
165) The method according to any of embodiments 144-151, wherein the at least one alteration is a substitution selected from the group consisting of R159K, R159H, R159Q, R159N, K165R, K165H, K165Q, K165N, Y294F, Y294Q, Y294K, Y294H, Y294N, K535R, K535H, K535Q, K535N, K590R, K590H, K590Q, K590N, K594R, K594H, K594Q, K594N, K649R, K649H, K649Q, K649N, N707S, N707A, N707G, N707L, D708S, D708A, D708G, D708L, F709Y, F709Q, F709K, F709H, F709N, Y723F, Y723Q, Y723K, Y723H, Y723N, R833K, R833Q, Y834F, Y834Q, Y834K, Y834H, Y834N, Y836F, Y836Q, Y836K, Y836H, Y836N, W927F, W927Q, W927K, W927H, W927N, R1118K, R1118H, R1118N, D1127N, D1127S, D1127A, T1128S, T1128V, T1128A, F1163Y, F1163Q, F1163K, F1163H, F1163N, R1167K, R1167H, and R1167N.
166) The method according to any of embodiments 144-151, wherein the at least one alteration is a substitution selected from the group consisting of 157S, M58S, L70S, L132P, G220D, L520A, F522Y, W531A, L669P, D708Y, N732S, K734N, L738A, E897S, Q898A, F901A, C1029A, F1031S, P1043L, and D1213N.
167) The method according to any of embodiments 144-151, wherein the at least one alteration includes W531A and P1043L; L699P; N732S, K734N, L738A, and D1213N; L70S and D708Y; L132P; C1029A and F1031S; N732S, K734N, and L738A; L520A, F522Y, E897S, Q898A, and F901A; 157S, M58S, N732S, K734N, and L738A; N732S, K734N, L738A, E897S, Q898A, and F901A; and G220D, N732S, K734N, and L738A.
168) The method according to any of embodiments 144-151, wherein the variant has a permissive temperature at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the permissive temperature is a temperature range selected from the group consisting of 25° C. or below, 26° C. or below, 27° C. or below, 28° C. or below, 29° C. or below, 30° C. or below, 31° C. or below, 32° C. or below, 33° C. or below, 34° C. or below, 35° C. or below, 36° C. or below, 37° C. or below, 38° C. or below, 39° C. or below, 40° C. or below, 41° C. or below, 42° C. or below, 43° C. or below, 44° C. or below, and 45° C. or below; preferably, the permissive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the permissive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the permissive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
169) The method according to any embodiments 144-168, wherein the variant has a restrictive temperature at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.; preferably, the restrictive temperature is a temperature range selected from the group consisting of 25° C. or above, 26° C. or above, 27° C. or above, 28° C. or above, 29° C. or above, 30° C. or above, 31° C. or above, 32° C. or above, 33° C. or above, 34° C. or above, 35° C. or above, 36° C. or above, 37° C. or above, 38° C. or above, 39° C. or above, 40° C. or above, 41° C. or above, 42° C. or above, 43° C. or above, 44° C. or above, and 45° C. or above; preferably, the restrictive temperature is a temperature range selected from the group consisting of 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.; preferably, the restrictive temperature is a temperature range selected from the group consisting of 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.; preferably, the restrictive temperature is a temperature range selected from the group consisting of 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.
170) The method according to any of embodiments 144-169, wherein the variant has a restrictive temperature that is higher than the permissive temperature.
171) The method according to any of embodiments 144-149, said method comprising the additional step of:

d) increasing the temperature to a restrictive temperature of the variant and cultivating the host cell, whereby the formed complex dissociates and expression of the one or more DNA target sequence is induced.

172) The method according to any of claims 116-171, wherein the one or more DNA target sequence comprises at least 20 nucleotides and further comprises or is flanked by a functional PAM sequence for a variant according to any of claims 1-7; preferably the one or more DNA target sequence is comprised in an open reading frame encoding a polypeptide or in a promoter region
173) The method according to any of embodiments 116-172, wherein the one or more DNA target sequence of interest encode one or more enzyme selected from the group consisting of hydrolase, isomerase, ligase, lyase, oxidoreductase, or a transferase; preferably the one or more enzyme is an alpha-amylase, alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucano-transferase, glucoamylase, invertase, laccase, lipase, mannosidase, mutanase, oxidase, pectinolytic enzyme, peroxidase, phosphodiesterase, phytase, polyphenoloxidase, proteolytic enzyme, ribonuclease, transglutaminase, and xylanase.
174) Use of a variant according to any of embodiments 1-22, a polynucleotide according to any of embodiments 23-44, a nucleic acid construct according to any of embodiments 45-66, an expression vector according to any of embodiments 67-88, a host cell according to any of embodiments 89-115, and/or a method according to any of embodiments 116-173 in medicine or cosmetics.
175) Use of a variant according to any of embodiments 1-22, a polynucleotide according to any of embodiments 23-44, a nucleic acid construct according to any of embodiments 45-66, an expression vector according to any of embodiments 67-88, a host cell according to any of embodiments 89-115, and/or a method according to any of embodiments 116-173 in medical or biotechnological research or production.
176) Use of a variant according to any of embodiments 1-22, a polynucleotide according to any of embodiments 23-44, a nucleic acid construct according to any of embodiments 45-66, an expression vector according to any of embodiments 67-88, a host cell according to any of embodiments 89-115, and/or a method according to any of embodiments 116-173 in genome editing, regulation of gene expression, or CRISPR inhibition.
177) Use of a variant according to any of embodiments 1-22, a polynucleotide according to any of embodiments 23-44, a nucleic acid construct according to any of embodiments 45-66, an expression vector according to any of embodiments 67-88, a host cell according to any of embodiments 89-115, and/or a method according to any of embodiments 116-173 in enzyme research, development, and/or production.

1-33. (canceled)

34: A temperature-sensitive variant of an RNA-guided endonuclease, wherein the variant has a sequence identity of at least 80% but less than 100%, to SEQ ID NO: 2, and wherein the variant comprises at least one alteration, preferably a substitution, deletion or insertion, of an amino acid important for stability of the RNA-guided endonuclease or for stability of a complex formed between the RNA-guided endonuclease, one or more guide-RNA (gRNA), and/or one or more DNA target sequence, wherein the at least one alteration is in a position corresponding to a position selected from the group consisting of 34, 51, 54, 57, 58, 60, 61, 65, 70, 82, 90, 112, 130, 131, 132, 134, 150, 154, 159, 164, 166, 174, 185, 188, 194, 197, 220, 244, 245, 287, 288, 294, 297, 344, 375, 448, 451, 455, 471, 507, 518, 520, 521, 522, 525, 531, 554, 571, 574, 586, 590, 649, 669, 681, 682, 707, 708, 709, 713, 715, 723, 732, 734, 738, 740, 747, 756, 833, 834, 836, 853, 860, 865, 876, 878, 897, 898, 901, 927, 929, 930, 932, 1004, 1017, 1011, 1027, 1029, 1031, 1042, 1043, 1101, 1118, 1127, 1128, 1163, 1196, 1197, 1200, 1209, 1210, 1212, and 1213 of SEQ ID NO: 2.

35: The variant according to claim 34, said variant being a nuclease-null variant; preferably said variant comprises an alteration of an amino acid corresponding to position 877 of SEQ ID NO: 2; more preferably said variant comprises a substitution of aspartic acid for alanine, D877A.

36: The variant according to claim 34, wherein the at least one alteration is in a position corresponding to a position selected from the group consisting of 70, 57, 58, 132, 220, 520, 522, 531, 669, 708, 732, 734, 738, 897, 898, 901, 1029, 1031, 1043 and 1213 of SEQ ID NO: 2.

37: The variant according to claim 34, wherein the at least one alteration is selected from the group consisting of F34S, F34N, F34A, F34L, R51K R51Q, R51S, R51A, R51G, R51L, L54S, L54A, L54G, L54Y, I57S, I57A, I57G, I57L, M58S, M58A, M58G, M58L, D60S, D60A, D60G, D60L, Y61S, Y61A, Y61G, Y61L, F65Y, F65L, F65S, F65A, F65G, L705, L70N, L70A, L70G, F82S, F82A, F82G, F82L, F82Y, K90Q, K90N, K90S, K90A, K112N, K112S, K112A, K112L, D130S, D130A, D130G, D130L, I131S, I131A, I131G, I131F, L132S, L132N, L132P, L132G, E134S, E134A, E134G, E134L, K150S, K150A, K150G, K150L, I154S, I154A, I154G, I154F, R159K, R159H, R159Q, R159N, F164S, F164A, F164G, F164Y, F164L, D166N, D166S, D166A, D166L, C174S, C174A, C174G, C185S, C185A, C185G, I188N, I188S, I188A, I188G, E194N, E194S, E194L, E194G, F197S, F197A, F197G, F197L, G220D, G220N, G220S, G220A, G220G, I244A, I244Y, I244H, I244G, T245A, T245Y, T245Q, T245G, L287A, L287N, L287S, L287G, C288S, C288A, C288G, Y294F, Y294Q, Y294K, Y294H, Y294N, P297L, P297S, P297A, P297G, P297Q, V344S, V344N, V344L, V344G, P375L, P375S, P375A, P375G, P375Q, E448A, E448A, E448G, E448L, A451D, A451N, A451S, A451K, A451G, K455N, K455S, K455A, K455L, F471S, F471N, F471A, F471G, Y507F, Y507L, Y507S, Y507A, Y507G, I518S, I518N, I518A, I518Y, L520S, L520N, L520A, L520Y, N521S, N521N, N521L, N521G F522Y, F522S, F522A, F522G, F522L, P525L, P525S, P525A, P525G, P525Q, W531F, W531S, W531A, W531G, W531L, I554S, I554A, I554G, I554L, P574L, P574S, P574A, P574G, P574Q, P586L, P586S, P586A, P586G, P586Q, K590R, K590H, K590Q, K590N, K649R, K649H, K649Q, K649N, D681H, D681S, D681A, D681G, D681L, W682S, W682A, W682G, W682L, N707S, N707A, N707G, N707L, D708S, D708A, D708G, D708L, F709Y, F709Q, F709K, F709H, F709N, L713S, L713A, L713G, L713N, L715S, L715A, L715G, L715N, Y723F, Y723Q, Y723K, Y723H, Y723N, N732S, N732A, N732H, N732L, K734N, K734S, K734L, K734G, L738S, L738A, L738G, L740S, L740A, L740G, L740N, F747S, F747A, F747G, F747L, P756L, P756S, P756A, P756G, P756Q, R833K, R833Q, Y834F, Y834Q, Y834K, Y834H, Y834N, Y836F, Y836Q, Y836K, Y836H, Y836N, K853R, K853Q, K853A, K853P, R860K, R860A, R860Q, I865A, I865F, I865S, I865N, I876S, I876A, I876Y, I876N, A877S, A877H, A877G, A877N, A877L, R878K, R878N, R878S, R878A, R878L, R878H, R878Q, E897S, E897A, E897G, E897L, Q898S, Q898A, Q898G, Q898L, F901S, F901A, F901G, F901L, W927F, W927Q, W927K, W927H, W927N, E929N, E929S, E929A, E929L, E929G, I930S, I930A, I930G, I930L, K932Q, K932N, K932S, K932A, N1004S, N1004A, N1004G, N1004L, Y1011S, Y1011A, Y1011G, Y1011L, P1017L, P1017S, P1017A, P1017G, P1017Q, C1027S, C1027A, C1027G, C1029S, C1029A, C1029G, F1031Y, F1031S, F1031A, F1031G, F1031L, D1042S, D1042A, D1042G, D1042L, P1043L, P1043S, P1043A, P1043G, P1043Q, W1101F, W1101L, W1101S, W1101H,W1101G, R1118K, R1118H, R1118N, D1127N, D1127S, D1127A, T1128S, T1128V, T1128A, F1163Y, F1163Q, F1163K, F1163H, F1163N, P1196L, P1196S, P1196A, P1196G, P1196Q, K1197Q, K1197N, K1197S, K1197A, D1200E, D1200N, D1200S, D1200A, D1200L, P1209L, P1209A, P1209A, P1209G, P1209Q, K1210N, K1210S, K1210A. K1210L, A1212N, A1212S, A1212G, A1212H, A1212L, D1213E, D1213N, D1213S, D1213L, and D1213G.

38: The variant according to claim 37, wherein the at least one alteration is selected from the group consisting of 157S, M58S, L70S, L132P, G220D, L520A, F522Y, W531A, L669P, D708Y, N732S, K734N, L738A, E897S, Q898A, F901A, C1029A, F1031S, P1043L, and D1213N.

39: The variant according to claim 37, wherein the at least one alteration is selected from the group consisting of W531A and P1043L; L699P; N732S, K734N, L738A, and D1213N; L70S and D708Y; L132P; C1029A and F1031S; N732S, K734N, and L738A; L520A, F522Y, E897S, Q898A, and F901A; 157S, M58S, N732S, K734N, and L738A; N732S, K734N, L738A, E897S, Q898A, and F901A; and G220D, N732S, K734N, and L738A.

40: A polynucleotide encoding a variant according to claim 34.

41: The polynucleotide according to claim 40, said polynucleotide having a sequence identity of at least 80% but less than 100%, to SEQ ID NO: 1.

42: A nucleic acid construct comprising a polynucleotide according to claim 40.

43: An expression vector comprising a polynucleotide according to claim 40.

44: A host cell comprising a polynucleotide according to claim 40.

45: The host cell according to claim 44, said host cell being a microorganism host cell selected from the group consisting of bacterial, fungal, yeast, and archaeal host cell.

46: The host cell according to claim 44, said host cell being a bacterial host cell selected from the group consisting of Bacillus, Escherichia, Lactobacillus, Lactococcus Streptococcus, and Streptomyces cell; preferably the host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, Bacillus thuringiensis, Escherichia coli, Lactobacillus acidophilus, Lactobacillus amylovorus, Lactobacillus brevis, Lactobacillus (para)casei, Lactobacillus cellobiosus, Lactobacillus crispatus, Lactobacillus curvatus, Lactobacillus delbrueckii subsp. bulgaricus, L. delbrueckii subsp. lactis, Lactobacillus fermentum, Lactobacillus gallinarum, Lactobacillus gasseri, Lactobacillus helveticus, Lactobacillus johnsonii, Lactobacillus plantarum, Lactobacillus reuteri, Lactobacillus rhamnosus, Lactobacillus salivarius, Lactococcus chungangensis, Lactococcus formosensis, Lactococcus fujiensis, Lactococcus garvieae, Lactococcus lactis, Lactococcus piscium, Lactococcus plantarum, Lactococcus raffinolactis, Lactococcus taiwanensi, Streptococcus equisimilis, Streptococcus pyogenes, Streptococcus uberis, Streptococcus equi subsp. zooepidemicus, Streptomyces achromogenes, Streptomyces avermitilis, Streptomyces coelicolor, Streptomyces griseus, and Streptomyces lividans cell; more preferably the host cell is a Bacillus licheniformis cell.

47: The host cell according to claim 44, said host cell being a fungal host cell selected from the group of consisting of Acremonium, Aspergillus, Aureobasidium, Bjerkandera, Ceriporiopsis, Chtysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chtysosporium inops, Chtysosporium keratinophilum, Chtysosporium lucknowense, Chtysosporium merdarium, Chtysosporium pannicola, Chtysosporium queenslandicum, Chtysosporium tropicum, Chtysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma Trichoderma longibrachiatum, Trichoderma reesei, and Trichoderma viride cell.

48: The host cell according to claim 44, said host cell being a yeast host cell selected from the group consisting of Candida, Hansenula, Kluyveromyces, Pichia, Saccharomyces, Schizosaccharomyces, and Yarrowia cell; preferably the host cell is selected from the group consisting of Kluyveromyces lactis, Pichia pastoris, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell.

49: A method of inducing expression of one or more DNA target sequence, the method comprising the steps of:
a) providing a host cell according to claim 44, said host cell further comprising one or more gRNA and one or more DNA target sequence;
b) cultivating the host cell at a permissive temperature of the variant and under conditions conducive for expression of the variant, whereby a complex is formed in the host cell between the variant with the one or more gRNA and the one or more DNA target sequence, and whereby expression of the one or more DNA target sequence is repressed; and subsequently
c) increasing the temperature to a restrictive temperature of the variant and cultivating the host cell, whereby the formed complex dissociates and expression of the one or more DNA target sequence is induced.

50: The method according to claim 49, said method comprising the additional step of:
d) decreasing the temperature to a permissive temperature of the variant, wherein a complex is formed in the host cell by the variant, the one or more gRNA, and the one or more DNA target sequence, and cultivating the host cell, whereby expression of the one or more DNA target sequence is repressed.

51: The method according to claim 49, wherein the permissive temperature is at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C., wherein the restrictive temperature is at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C., and wherein the restrictive temperature is higher than the permissive temperature.

52: The method according to claim 49, wherein the one or more DNA target sequence comprises at least 20 nucleotides and further comprises or is flanked by a functional PAM sequence for a variant according to claim 34.

53: The method according to claim 49, wherein the one or more DNA target sequence encode one or more enzyme selected from the group consisting of hydrolase, isomerase, ligase, lyase, oxidoreductase, or a transferase.

54: The method according to claim 49, wherein the host cell is a fungal, bacterial, or archaeal cell.

55: The method according to claim 49, wherein the host cell is a Bacillus host cell.

56: The method according to claim 49, wherein the one or more gRNA comprises a first RNA comprising 20 or more nucleotides that are at least 85% complementary to and capable of hybridizing to the one or more DNA target sequence.

57: A method of repressing one or more DNA target sequence, the method comprising the steps of:
a) providing a host cell according to claim 44, said host cell further comprising one or more gRNA and one or more DNA target sequence;
b) cultivating the host cell at a restrictive temperature of the variant and under conditions conducive for expression of the variant, wherein the one or more DNA target sequence is expressed; and subsequently
c) decreasing the temperature to a permissive temperature of the variant and cultivating the host cell, whereby a complex is formed in the host cell between the variant, the one or more gRNA and the one or more DNA target sequence, and whereby expression of the one or more DNA target sequence is repressed.

58: The method according to claim 57, said method comprising the additional step of:
d) increasing the temperature to a restrictive temperature of the variant and cultivating the host cell, whereby the formed complex dissociates and expression of the one or more DNA target sequence is induced.

59: The method according to claim 57, wherein the permissive temperature is at or below a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C., wherein the restrictive temperature is at or above a temperature selected from the group consisting of 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C., and wherein the restrictive temperature is higher than the permissive temperature.


SEQUENCE LISTING (19 sequences)

SEQ ID NO: 1 - DNA, 3792 bp, Artificial Sequence
  Mad7 DNA sequence, codon-optimized for B. licheniformis
atgaataatggcacaaataacttccagaacttcattggcattagcagcctgcaaaaaacactgagaaatgcactgattccgacagaaacaacacagcagtttattgtcaaaaacggcatcatcaaagaggatgaactgagaggcgaaaatcgccaaattctgaaagatatcatggacgactattaccgtggctttatttcagaaacactgtccagcattgatgatatcgattggacaagcctgttcgagaaaatggaaatccaactgaaaaacggcgataacaaagacacgctgattaaagaacaaacggaatatcgcaaagcgatccacaaaaagtttgcaaatgatgaccgctttaaaaacatgttcagcgcgaaactgattagcgatattctgccggaatttgtcatccacaataataactatagcgcgagcgagaaagaagaaaaaacacaggtcattaaactgtttagccgctttgccacaagcttcaaagactatttcaaaaatcgcgcaaactgctttagcgcagatgatatttcatcatcaagctgccatcggattgtcaatgataatgcggaaatcttttttagcaacgcactggtctatcgcagaattgttaaatcattgagcaacgacgacatcaacaaaatctcaggcgatatgaaagacagcctgaaagaaatgtcactggaagaaatctacagctacgaaaaatacggcgaatttatcacacaagaaggcatcagcttttacaacgatatttgcggcaaagtcaacagctttatgaatctgtattgccagaaaaacaaagaaaacaaaaacctgtataaactgcagaaactgcacaagcagattctgtgcattgcagatacatcatatgaagtcccgtacaaatttgagagcgacgaagaagtttatcaaagcgttaatggctttctggataacatcagcagcaaacatattgttgaacgcctgagaaaaattggcgataactataatggctacaacctggacaaaatctacatcgtcagcaaattttacgaaagcgtcagccaaaaaacatatcgcgattgggaaacaattaatacagcgctggaaattcattataacaacattctgcctggcaacggcaaaagcaaagcagataaagttaaaaaggcggtcaaaaatgacctgcagaaaagcattacagaaatcaatgaactggtcagcaactacaaactgtgctcagatgataatatcaaggcggaaacgtacatccatgaaattagccatatcctgaacaactttgaagcgcaagaactgaaatataacccggaaatccatctggttgaaagcgaactgaaagcaagcgagctgaaaaatgttctggatgtcattatgaatgcgtttcattggtgcagcgtctttatgacagaagaactggtcgataaagataacaacttttatgcggaactggaagagatttacgacgaaatttatccggtcatcagcctgtataatctggttcgcaattatgtcacacagaaaccgtatagcacgaagaaaatcaaactgaactttggcattccgacactggcagatggctggtcaaaatcaaaagaatatagcaacaacgcgatcatcctgatgcgcgataatctttattatctgggcattttcaacgcgaaaaacaagccggacaaaaaaatcatcgaaggcaatacgtcagagaacaaaggcgactataaaaagatgatctataatctgcttccgggaccgaataaaatgatcccgaaagtttttctgtcaagcaaaacaggcgtcgaaacatataaaccgtcagcgtatattctggaaggctacaaacagaacaaacacatcaaaagcagcaaggactttgacatcacattttgccatgatctgatcgactactttaagaactgcattgcaattcatccggaatggaaaaacttcggctttgatttttcagacacgagcacgtatgaagatatcagcggcttttatagagaagttgaactgcagggctataaaatcgactggacatatatcagcgaaaaggatattgatctgctgcaagaaaaaggccaactgtacctgtttcagatctacaacaaagacttcagcaaaaaaagcacgggcaatgataacctgcatacgatgtacctgaaaaacctttttagcgaagagaacctgaaagacattgtcctgaaactgaatggcgaagccgaaattttctttcgcaaatccagcattaaaaacccgatcatccataaaaaaggcagcattctggttaaccgcacatatgaagcggaagaaaaagatcagtttggcaacattcagatcgtccgcaaaaacattccggaaaacatttatcaagaactgtacaaatactttaacgataaaagcgataaagaactgtccgacgaagcagcgaaacttaaaaatgttgttggccatcatgaagcggcaacaaacattgttaaagactatcgctatacgtacgataaatactttctgcatatgccgatcacgatcaacttcaaagcaaataaaacgggctttatcaacgatcgcattctgcagtatattgccaaagaaaaggatctgcatgtcatcggcattgatagaggcgaacgcaatctgatttatgtcagcgttattgatacatgcggcaacattgtcgaacagaaaagctttaacattgtcaacggctatgactaccagatcaagctgaaacagcaagaaggcgcaagacaaattgctcgcaaagaatggaaagaaatcggcaagatcaaagaaattaaagagggctatctgagcctggtcattcatgaaatttctaaaatggtcatcaaatataacgcgattatcgccatggaagatctgtcatatggctttaagaaaggccgttttaaagtcgaaagacaggtctaccagaaattcgaaacaatgctgattaacaaactgaattatctggtgtttaaagacatcagcatcacggaaaatggcggactgctgaaaggctatcaactgacatatattccggataagcttaaaaacgtcggccatcaatgcggctgcatcttttatgttccggcagcgtatacatcaaaaattgatccgacaacaggctttgtcaacatcttcaaattcaaagatctgacggtcgatgcgaaacgcgaattcattaagaaatttgacagcatccgctacgacagcgagaaaaatcttttctgctttacgttcgactacaacaactttatcacgcagaatacggttatgtcaaaaagcagctggtcagtctatacatatggcgttagaattaaacgcagatttgtgaacggcagatttagcaatgaaagcgatacaatcgacatcacgaaagacatggaaaaaacgcttgaaatgacggatattaactggcgtgatggacatgatcttcgccaggatattatcgattatgaaatcgtccagcacatctttgaaatctttagactgacagtccaaatgcgcaattcactgtcagaacttgaagatagagattatgatcgcctgatttctccggtcctgaatgaaaataacatcttttacgatagcgcaaaagcaggcgacgcactgccgaaagatgcggatgcaaatggcgcatattgcattgcactgaaaggcctgtatgaaatcaaacaaatcaccgagaattggaaagaggacggcaaattttcacgggataaactgaaaatcagcaacaaggactggtttgacttcatccaaaataagcgctacctgtaa

SEQ ID NO: 2 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 3 - DNA, 9936 bp, Artificial Sequence
  MOL2212-amyL locus
tctagaccttctttgtgcttggaagcagagcccaatattatcccgaaacgataaaacggatgctgaaggaaggaaacgaagtcggcaaccattcctgggaccatccgttattgacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgttcgcagatgctgctgaagagattattaaaaagctgaaagcaaaaggctatcaattggtaactgtatctcagcgcatgcagcaggtagttctatcaaaccgtgaattgacagtatatagagaagatatttctcgcctggctgatcaagaacagcagacgctgattgatgcttttatgactaaggatcgggaaaaaggatttgatttgcaaaaagatcctttaatgcgtcttgccctttttgatagaggagacagccaatatacatgtgtctggacacatcaccatatcatcatggatgggtggtgtctcggcattattcttaaagagtttttcagcatgtatgattcgctcaaaaataactcacctgtacagcttggcagcacggtgccgtacagccgttatattgaatggctcggagaacaagatcaagaagagactgctgcctactggagcgaatatttgaaggagtacggcaatactgcttctattccccgaataaagcgccgcacggcagacgggaattataaagccgaccaggtcagcttttcattagcgccggatatggttgagaaactgacagaggctgcccaaaactggggagtgacattaaacacgctgtttatgagtatttggggcgtgctgctccatcgatataatgctgcagatgatgccgtttttggctccgtcatttcgggacgcccgtcagcgattgacgggattgaatcaatggtcggtttgtttatcaatactgttccagtgcgaatccgatctgcggaaggcataacattttcttcgctggtcaaagccgtgcaggaggatattttatcatctgaacagcatgggtattatccgctttatgagattcaaaaccatagtccattgaagcaagggttgatcgatcatatttttgtttttgaaaattaccctgtacagcttcatcaggcattaagcgtggagagtgaaaatgacgaaggcgctctgaagctgagtgacatttcaatgtcggaacaaaccaattacgactttaatatcgttatcgttccgggagaatcattttatatcaaattcagctacaacgcagacgtttacgagcgggaggagatgctgaggattcaggggcatctaaagcaagcactggattgcattttgacaaatcccgatgttgccgtcagtgacatcaatatcgtgccgccggaggagcaacaggttatccaattgtttaacgaaactgagcgtccttatgtgaacaaaacgataccccaattgtttgaggaacaagctcacaaaacacctgaagctgctgcattgaaaatgggaaacgagtgctggacctatcgccagttacaagtaagagccaatcagattgcgcacgccctgatagaaaaaggcgttggatctggggatatcgttgctgtaatgatgggccggtcaatggaaatgcctgcagctctgcttgggatttggaaagcaggcggcgcttatatgccgcttgatccacattttccagcagaacgtctttcttttcttctgaaggacagtcaagcggctcaattgctgatagaagaagaccttatttccttgatccctccgtcctatgaaggaaatacgataacgatagaacatacagaaagttaccaaacagaagcgccaaatatgccgccaggtgatctagcctacttgatctatacgtcgggaacgacggggcgccctaaaggggttttagttgatcatcacggtattgccaatacattgcaatggagacgggaagagtatagcatgaccgaacaggatatatccctccatttgttttcgtacgtgtttgacggctgtgtaacgagcttatttaccccgcttttatctggtgcgtgcgtactgctgacaacagatgacgaagcgaaggatgtgctggcccttaagcgaaaaatagcccgttataaggtcagccacatgatcattgttccttccctgtacagggtgttattggaagtgatgactgctgacgatgcaaaaagtcttcgtattgtgacatttgcgggtgaagcggtcacgcctgatctgcttgagttgaatcaaataatttgtccttctgctgaactggcaaatgaatacgggcccacagaaaacagtgtggcaacaacaatattgcggcatctgaataaaaaagagaggatcacgatcggacacccgatcagaaacacaaaagtatttgttttgcacggaaatcaaatgcagccgatcggcgcggcgggtgaactgtgtatttccggcgcgggtcttgcgagaggatactacaaacagcaagagctgacacagaaagcattctccgatcatccattccttgaaggggagcgtttataccgaacaggtgatgcaggccgctttttgcctgatggaacgattgaatatattggacgttttgatgatcaagtgaaaattaggggttatcgtattgaactgagagagattgaaacagttcttcgacaagcaccgggggtaaaagaagcagcggtactggcccgtgatgtttctgctgaggaaaaggagctcgttgcttatatcgttccggaaaagggaaacagcctcccagatttgtatcagcatcttgccgggacattgccgtcctatatgatcccagcaagtattatcaacatcagccagatgccattaacatccagcggcagatcacccgcgataccgtcattttcgacacatttctttctttgctacatcagataacgttgccatttcatccccgccttacctagggattctaaactgtcagcaatattcctgagggcttatgacactttgttaaaattaattataaaatgtaatcaacgaaatttataagacgggcaaaataaaaaaacggatttccttcaggaaatccgtcctctctgctcttctatctttgaacataaattgaaaccgacccgccgtttacgtgaaactctccccagccttccgaattgatgacaaccggctccgaacggtttccggtaatgtcatgccatgtctcaccggcgttttgccggccgacatacattcgctttgccccaccgggtccgtctgttattaatgccgccaaacctgaatttgcaaccgagctgtcgccttcccttgtccagccgacaatgtcatggtggtcgaaataatcatgctgtgctccgtacgcatactgttttctcgcttttaagatcggttcaattttgtgtttcaaggcaggaatttcgcgctgggagtctcctttcgtcccgtacatatccccgtagaaaacctgagggtatccagattcccttgtgagaataaaagcgtaagcaagcggcttaaaccatgtttggacagtcgactcaagcgattgccccggctgtgtatcatggttatcgacaaatgtaaccgatttcaacggatgcttggaaacgaccgtacccttcagcaatttcctcatatcatagccgcctccctgtgtcgatgcagcatggaactgataatgaagcggcacgtcaaacactgaatgattaaaatttgttttgttcaaatagttttccagcgcgcccaagtcattctgccaatattcagctaccgtaaacatttccttccccgttttttccctgacatgattaacccaatcccgcaaaaaagaaaatttaatgtgtttgacagcatcaagacggtttccgtccaattgcagttcattggcataccaagtgccccatctcttaatttctgctgcgacatcaggatggtcataatcgatgtcggcatacatcaaataatcatagttgccgttttcattggaaacttcccaatcccaagcctttccttgaaacttatagatgcggttcagctttcgggactcgtcccaatcggttccgtcaaaatggtaccaatgccatttaaaatcgctgtatgtgctgccggcccccggaaaatgaaaatgtgtccaggctttaattaggtgttctcctgagattacgcggttgcggtcagcgggatcgacttcaaccgcggttacatcttcggtcgcatcagcgccgcctttgtggttgatgaccacatccccgtaaacgttaatgtcgcgggaatgaagacttttgatcgcagattgcagctctccttttgtgccgtactttgtccgaaccgtccctttttgatgaaactcccctaaatcataaaggtcgtaagcaccgtagcccacatccgcttggctcgttcccttatatgccgggggaatccagacggcagtaataccgtgttcagccaaatatgccgagtcgttttgcaaacgcttccaatgttggccgtcattgggcatgtaccattcaaaatactgcatcagcgtcccattaagatttgccgccgctgctgcagaatgaggcagcaagaagatgagcgcaaataacagcgtcagcaatcgggcgtaaagccgtttttgttgtttcatgattctcctcccctttcaatgtgatacatatgatattgtataaatattccgaatttttaacaagtaccattttccctatattttcttccaagatctgtcgaccgcggctagagcggccgcacgcgtgctagccatggcttttacaatagaaggaaaagtcaccccgcagttttttcaaaacagcatcaacgctcttgtagaaagacatgatattttcagaacgatttttatcagccaaaatgtttcctccccccagcaggttgttctgagagaacgaaatgtcatcgtactggaagaagacattactcatttaaacgaggcggagcaatcacagtttatcgagcaatggaaagaaaaggaccgggaccgggggtttcatttgcaaaaggatgtgctcatgcgcatcgctctgattcaaacaggagagagccagtactcttgcatttggacctttcatcatatcatgatggacggttggtgcctcagcattgtgctaaaggagtttctgcatatttacgcatcttatgtaaatgcatccccaataacattggagccggtccagccatacggaaagtatatcaaatggctgatggagcaagacaaagagcaggcggtttcttattgggatcattatctttccggccacgaacagcaaaccgtgctcccgaagcagaaaaaaacaaagggaaaaagcaggcaggaacacgttacattttcattctctaaggaagaaagcagcaggctttcagagcttgctgctagagaagaagtgacactcagtacgatttttcataccatttggggcattctgctgcaaaaatataataataacgatgacgccgtattcggttctgtcatttcaggaagacccgcagaaatcgaaggcattgaacatatggtaggtcttttcattaatacgatgccggttcgggtgcagggggcaaaaacgccttttcttcaacttataaaagatatgcaaaaagaccgtcttgccgctgaagcgtacagctaccaccctttatatgagattcaatcccgctcagctgttaagcaagggctgattgatcatattctcgtatttgaaaactatccggtgcagcaagagattcaaatgctgaacaaacaggaacatgcttctgatctttttcagattcataatttcactgtcgcagatgaaaccaactacagcttttatcttatggtggcgccaggggaagaaattcatatcaaaatgaactatgatgcggagcagcacgatcggtcttttgtgctctctgtcaaagagcatcttctgaatgctgtatcacaaatcttaaataatccgaatcttccacctgaagaaatcgatatcacgacagatactgaaaagcggcagctgattggagaaatcacagatcaaacaccagtttacgaaaccatccatgccatgtttgaaaagcaagcggaaaagacaccagatgctcatgctgtaattgatcaagcctgctcattaacatacagagaactgaataaagcggctaacagattggcacggcatttacgaatgaaaggcgttgtgagacaggaacccgttgcgattatgatggaacgctcagcggcgtttatcacaggtgttcttggtatcttgaaagcaggcggcgccattgttccggttgatccgcattaccctgctgacagaatccgttatattctgcatgattgcggctgttcgcatgttgtttcacaagcgcatcttccctcatcgttagaagacaattacattatcactcatccagaagatatcgaaagcaaagtagacggcagtaacataaagtcagtcaacaatgctgacgatctgctgtatatgatttatacatcaggcacaacaggtaaaccaaagggtgttcaatttgagcatcgaaacatggctaacttgctgaagttcgaatatactcactccggcattgactttgaagcagatgttctgcaatttgcgacgccttccttcgacgtctgctatcaagaaatattttctgcgcttctgaaaggcggcacactccacatcgtgccagaagccataaaaagagatgtgcctcagctgtttgcatttataaacaagcatcagacgaatattgtgtttctcccaactgcttttatcaaaatgattttcagcgagcgagaacttgcgaactcgtttcctgatggcgtcaaacacctgatcgcagccggggaacaattaatgatttcagaccttttccaagatgtgttgcgtaaacgcggcatacacctgcacaatcattacggaccttcagaaactcatgtggtatctacgtataccattcatcctggagaccctattcctgagcttccgccaatcggtaaaccgataggctgtactgatctatatattttgaatcatcaaaaacagcttcagccatgcggcgtaccaggcgaactctatatttccggcgcaagtgttgccagaggatatgtcaatcacgataaactgacaagcgacaaattttcatccgatccattcaagcctgatgtgatcatgtaccggacaggtgatttggccagacggcttgaagacggcaacatcgaatatatcggacgggcagacaatcaagtgaaaatcagaggttatcgaattgagccgcaagaaatcgaagtcacgctaatgaatcatccggacattagtgaggctgccattctcatctggcaagatcaaaacggtgaacatgaattatgcgcttactattgcagtgtacaaaagctgaacacaatagatttacgcagttatatggctagtgagctcccagaatacatgattccggcgaaatggatatgggttgacagcatacctcttaccccaaacggaaaagtagatcgcgcagcacttccggagccagatgcttcaatcagcggaaacccatatactgcccctcgaaatctgttagaggccatggctagcacgcgtgcggccgccgcggtcgacagatcttggaagaaaatatagggaaaatggtacttgttaaaaattcggaatatttatacaatatcatatgtatcacattgaaaggggaggagaatcatgaaacaacaaaaacggctttacgcccgattgctgacgctgttatttgcgctcatcttcttgctgcctcattctgcagcagcggcggcaaatcttaatgggacgctgatgcagtattttgaatggtacatgcccaatgacggccaacattggaagcgtttgcaaaacgactcggcatatttggctgaacacggtattactgccgtctggattcccccggcatataagggaacgagccaagcggatgtgggctacggtgcttacgacctttatgatttaggggagtttcatcaaaaagggacggttcggacaaagtacggcacaaaaggagagctgcaatctgcgatcaaaagtcttcattcccgcgacattaacgtttacggggatgtggtcatcaaccacaaaggcggcgctgatgcgaccgaagatgtaaccgcggttgaagtcgatcccgctgaccgcaaccgcgtaatctcaggagaacacctaattaaagcctggacacattttcattttccgggggccggcagcacatacagcgattttaaatggcattggtaccattttgacggaaccgattgggacgagtcccgaaagctgaaccgcatctataagtttcaaggaaaggcttgggattgggaagtttccaatgaaaacggcaactatgattatttgatgtatgccgacatcgattatgaccatcctgatgtcgcagcagaaattaagagatggggcacttggtatgccaatgaactgcaattggacggaaaccgtcttgatgctgtcaaacacattaaattttcttttttgcgggattgggttaatcatgtcagggaaaaaacggggaaggaaatgtttacggtagctgaatattggcagaatgacttgggcgcgctggaaaactatttgaacaaaacaaattttaatcattcagtgtttgacgtgccgcttcattatcagttccatgctgcatcgacacagggaggcggctatgatatgaggaaattgctgaagggtacggtcgtttccaagcatccgttgaaatcggttacatttgtcgataaccatgatacacagccggggcaatcgcttgagtcgactgtccaaacatggtttaagccgcttgcttacgcttttattctcacaagggaatctggataccctcaggttttctacggggatatgtacgggacgaaaggagactcccagcgcgaaattcctgccttgaaacacaaaattgaaccgatcttaaaagcgagaaaacagtatgcgtacggagcacagcatgattatttcgaccaccatgacattgtcggctggacaagggaaggcgacagctcggttgcaaattcaggtttggcggcattaataacagacggacccggtggggcaaagcgaatgtatgtcggccggcaaaacgccggtgagacatggcatgacattaccggaaaccgttcggagccggttgtcatcaattcggaaggctggggagagtttcacgtaaacggcgggtcggtttcaatttatgttcaaagatagaagagcagagaggacggatttcctgaaggaaatccgtttttttattttgcccgtcttataaatttctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattattgc

SEQ ID NO: 4 - DNA, 3276 bp, Artificial Sequence
  MOL2212-xyl locus
  misc_feature (3139)..(3139): n is a, c, g, or t
gaattcaaaagccgcttccgccctggctttcgctttatccaaaggatgtgtcagccggttccacgcccggaacatcgtcccgtcgccgaaagggtctttcccgtccgcatcaaaggtatgccaatacgcaacagcgaagcgaaggtgttccttcatcgttttgccgccgacaaattcatcagggttgtaatatttaaatgcgtaaggattttccgaatcggccccttcatactcaatcattccgatatttctaaaaaacattccgatctcccccttcacttttccttgcaaacgttaaaaaacaatgtttgtttaaccattaaactaacttccttgtatgtattttacaggatcaattaatcgctttcaatggaaatagccgcggatcgatgctagcagatcttggaagaaaatatagggaaaatggtacttgttaaaaattcggaatatttatacaatatcatatgtatcacattgaaaggggaggagaatcatgaaacaacaaaaacggctttacgcccgattgctgacgctgttatttgcgctcatcttcttgctgcctcattctgcagcagcggcggcaaatcttaatgggacgctgatgcagtattttgaatggtacatgcccaatgacggccaacattggaagcgtttgcaaaacgactcggcatatttggctgaacacggtattactgccgtctggattcccccggcatataagggaacgagccaagcggatgtgggctacggtgcttacgacctttatgatttaggggagtttcatcaaaaagggacggttcggacaaagtacggcacaaaaggagagctgcaatctgcgatcaaaagtcttcattcccgcgacattaacgtttacggggatgtggtcatcaaccacaaaggcggcgctgatgcgaccgaagatgtaaccgcggttgaagtcgatcccgctgaccgcaaccgcgtaatctcaggagaacacctaattaaagcctggacacattttcattttccgggggccggcagcacatacagcgattttaaatggcattggtaccattttgacggaaccgattgggacgagtcccgaaagctgaaccgcatctataagtttcaaggaaaggcttgggattgggaagtttccaatgaaaacggcaactatgattatttgatgtatgccgacatcgattatgaccatcctgatgtcgcagcagaaattaagagatggggcacttggtatgccaatgaactgcaattggacggaaaccgtcttgatgctgtcaaacacattaaattttcttttttgcgggattgggttaatcatgtcagggaaaaaacggggaaggaaatgtttacggtagctgaatattggcagaatgacttgggcgcgctggaaaactatttgaacaaaacaaattttaatcattcagtgtttgacgtgccgcttcattatcagttccatgctgcatcgacacagggaggcggctatgatatgaggaaattgctgaagggtacggtcgtttccaagcatccgttgaaatcggttacatttgtcgataaccatgatacacagccggggcaatcgcttgagtcgactgtccaaacatggtttaagccgcttgcttacgcttttattctcacaagggaatctggataccctcaggttttctacggggatatgtacgggacgaaaggagactcccagcgcgaaattcctgccttgaaacacaaaattgaaccgatcttaaaagcgagaaaacagtatgcgtacggagcacagcatgattatttcgaccaccatgacattgtcggctggacaagggaaggcgacagctcggttgcaaattcaggtttggcggcattaataacagacggacccggtggggcaaagcgaatgtatgtcggccggcaaaacgccggtgagacatggcatgacattaccggaaaccgttcggagccggttgtcatcaattcggaaggctggggagagtttcacgtaaacggcgggtcggtttcaatttatgttcaaagatagaagagcagagaggacggatttcctgaaggaaatccgtttttttattttgcccgtcttataaatttcgttgattacattttataattaattttaacaaagtgtcataagccctcaggaatattgctgacagtttagaatccctaggtaaggcggggatgaaatggcaacgttatctgatgtagcaaagaaagaaatgtgtcgaaaatgacggtatcgcgggtgatctgcggccgcacgcgtgtcgacagtactgaaatagaggaaaaaataagttttcaaaatttgttatactataataaattcacagcttcaaagcaatggagtgattgacattgaatacagcggatcaagcccttgtaaaaaaaatgaacaaagccctgatttttgagcagattattgaaaacggacccgtctcaagggccaaactttcggaaataacaggattaaataaagcgaccgtttcctcccaagtgtcttccctgcttcaaaaggacatcatttatgaaaccggccccggcgagtcaagcggcggcagaaggcccgttatgctgaaattcaatcgaaaagccggctatgcggtcggtgtagatgtcggaaccaattatataattgttgcgttaaccgaccttgaagggcatttgattgaacaattcgaacgaacgcttgatgaagaagacattcaggcgacagaagaggcattaatcgaattgacggggcttgccgttgacaaaataccgccttccccattcggattgaccggaatcggcgtgtgcgtgcccggcttggtggacaacgaacggcatgtagtctttacgccaaataagccgatccatttgattccgataaaagaaaaactggaagaaaggttcggcgttcccattttaattgaaaacgaagccaacgccggcgccgttggcgagaaggaatacggggaaggcggccagcttgagcatgccgtgtttgtcagcattaataccgggatcggtttagggattttaatgaatggaaaattgtttagaggtgtgcaaggtttttccggagaagccgggcacatgtccattcactttgacggtccgttatgccggtgcggaaatagaggctgctggnagctctacgcttcagaaaaagccgtcttctctcactatgcagaagcttgcatgcctgcaggtcgattcacaaaaaataggcacacgaaaaacaagttaagggatgcagtttatgcatcccttaacttacttattaaa

SEQ ID NO: 5 - DNA, 3521 bp, Artificial Sequence
  MOL2212-gnt locus
gcagcggttcgtttaatcgggaatgcctcgacttcgatggtcatctccctgttggtcgcgatctatacgatggggatcgccagaaagatcccgatcaaacaagtgatggattcctgttcaaccgccattacacaaatcggaatgatgctcttgatcatcgggggcggcggcgccttcaaacaagtcttgatcaatggcggagtaggcgactatgtagctgaattattcaaaggaacagccatgtcgccgatcttgctcgcctgggtcatcgccgcaattctgcgcatctccttaggatccgcgacagttgctgcattaagcacaaccggactcgttcttccgatgctcggacaaagcgatgtcaatcttgcgctggttgtgcttgcaacaggggccggaagtgtaatcgcttcccatgtcaatgacgcaggcttctggatgttcaaagagtatttcggactaagcatgaaagagacatttgccacctggacactgcttgaaaccattatcgcggtggccggattaggatttactttattgttaagcctatttgtataaaaaagatcacccgcgataccgtcattttcgacacatttctttctttgctacatcagataacgttgccatttcatccccgccttacctagggattctaaactgtcagcaatattcctgagggcttatgacactttgttaaaattaattataaaatgtaatcaacgaaatttataagacgggcaaaataaaaaaacggatttccttcaggaaatccgtcctctctgctcttctatctttgaacataaattgaaaccgacccgccgtttacgtgaaactctccccagccttccgaattgatgacaaccggctccgaacggtttccggtaatgtcatgccatgtctcaccggcgttttgccggccgacatacattcgctttgccccaccgggtccgtctgttattaatgccgccaaacctgaatttgcaaccgagctgtcgccttcccttgtccagccgacaatgtcatggtggtcgaaataatcatgctgtgctccgtacgcatactgttttctcgcttttaagatcggttcaattttgtgtttcaaggcaggaatttcgcgctgggagtctcctttcgtcccgtacatatccccgtagaaaacctgagggtatccagattcccttgtgagaataaaagcgtaagcaagcggcttaaaccatgtttggacagtcgactcaagcgattgccccggctgtgtatcatggttatcgacaaatgtaaccgatttcaacggatgcttggaaacgaccgtacccttcagcaatttcctcatatcatagccgcctccctgtgtcgatgcagcatggaactgataatgaagcggcacgtcaaacactgaatgattaaaatttgttttgttcaaatagttttccagcgcgcccaagtcattctgccaatattcagctaccgtaaacatttccttccccgttttttccctgacatgattaacccaatcccgcaaaaaagaaaatttaatgtgtttgacagcatcaagacggtttccgtccaattgcagttcattggcataccaagtgccccatctcttaatttctgctgcgacatcaggatggtcataatcgatgtcggcatacatcaaataatcatagttgccgttttcattggaaacttcccaatcccaagcctttccttgaaacttatagatgcggttcagctttcgggactcgtcccaatcggttccgtcaaaatggtaccaatgccatttaaaatcgctgtatgtgctgccggcccccggaaaatgaaaatgtgtccaggctttaattaggtgttctcctgagattacgcggttgcggtcagcgggatcgacttcaaccgcggttacatcttcggtcgcatcagcgccgcctttgtggttgatgaccacatccccgtaaacgttaatgtcgcgggaatgaagacttttgatcgcagattgcagctctccttttgtgccgtactttgtccgaaccgtccctttttgatgaaactcccctaaatcataaaggtcgtaagcaccgtagcccacatccgcttggctcgttcccttatatgccgggggaatccagacggcagtaataccgtgttcagccaaatatgccgagtcgttttgcaaacgcttccaatgttggccgtcattgggcatgtaccattcaaaatactgcatcagcgtcccattaagatttgccgccgctgctgcagaatgaggcagcaagaagatgagcgcaaataacagcgtcagcaatcgggcgtaaagccgtttttgttgtttcatgattctcctcccctttcaatgtgatacatatgatattgtataaatattccgaatttttaacaagtaccattttccctatattttcttccaagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctctaataattgttaatcatgttggagctcagtgagagcgaagcgaacacttgattttttaattttctatcttttataggtcattagagtatacttatttgtcctataaactatttagcagcataatagatttattgaataggtcatttaagttgagcatattagaggaggaaaatcttggagaaatatttgaagaacccgagaaaaatctttcttatggtcacagcaggcaaaccggttgattctgttatcgactcgctcgttcctctgcttgaagaaggggatgtcatcatggacggcggcaactcccactacgaagatacggaacgaagatatgacagtttgaaagcgaaaggtatcggctacttgggaatcggcatttccggcggcgaagtcggcgctttaaaaggcccatccattatgcccggcggagatcgggacgtctacgaaaaagcagctccgattctcacaaaaatcgccgcccaagtagaaggagatccatgctgtgtctacatcggtccaaaaggcgcggggcattttgttaaaatggtgcacaacggcatcgaatacgcagacatgcagcttatcgctgaagcgtatacatttttaagagaaaagcttcttttgccgatagatgaaatcgctgacattttcgacacgtggaatcaaggagagctgaaaagctatttaatcgaaatcacggcagagatcctgaggaaaaaggatgagcgaacgggcgctccgctcatcgacgtcatcctcgacaaaaccggccaaaaaggcacgggcaaatggacgagcctgcaggccatcgacaacggcattccatcatcaattatcacggaatccctgtttgcccgttacctgtcatcattaaaagacgaacggacagctgccgaaaacgtattggccgggcctgaaacagaagaac

SEQ ID NO: 6 - DNA, 2323 bp, Artificial Sequence
  MOL2212-prsA locus
  misc_feature (1005)..(1005): n is a, c, g, or t
  misc_feature (1390)..(1390): n is a, c, g, or t
aattcattccgccttttgaagcggagtagtgaacatttttcggtctcggaatttgctgatgaacgctggatatattgagtatgcttcctttcatgcggcggtccgccatatattttaaggcttcgcgtgcgcagagaaacgcaccggttaaattaacgtcgatcactttctgccactcatcaagctcaagttcgtgcggaaaggcgcccgcgccgttaaatcctgaattattgaccataacatcaagtgaaccaaagtgttcaactgctgaattaatcagctttttcacgtcctcttcggaagacacgtcagcttctatcgcaagggcgtttccgcccgctgcttgaatattgtcgacggcttcccgtgcgcctccgggatcgctgtgataattgacaaccacattcatcttttcttttccaaaccgttctgcaaccgccttgcctataccttttgaagagccggtcacaattgctgtttttccttttaaatcactatacaacctaaacacccctcaatttcttttctccatgtacattacccggtatcagctagcagatccatgagtagaagcgccatatcggcgcttttcttttggaagaaaatatagggaaaatggtacttgttaaaaattcggaatatttatacaatatcatatgttacacattgaaaggggaggagaatcatgaaacaacaaaaacggctttacgcccgattgtgaattcataattcccctctgttgaaaatctttttacagcatgtcagaatatgatatgatacaattcaaaggaaagtttaaactgttatgattaggagtgtttgcatttatgaagaagattgcaattgcggcgattacagcgacaagcgtgctggctctcagcgcatgcagcgggggagattctgaggttgttgcggaaacaaaagctggaaatattacaaaagaagacctttatcaaacattaaaagacaatgccggagcggacgcactgaacatgcttgttcagnaaaaagtactcgatgataaatacgatgtctccgacaaagaaatcgacaaaaagctgaacgagtacaaaaaatcaatgggtgaccagctcaaccagctcattgaccaaaaaggcgaagacttcgtcaaagaacagatcaaatacgaacttctgatgcaaaaagccgcaaaggataacataaaagtaaccgatgatgacgtaaaagaatattatgacggcctgaaaggcaaaatccacttaagccacattcttgtgaaagaaaagaaaacggctgaagaagttgagaaaaagctgaaaaaaggcgaaaaattcgaagaccttgcaaaagagtattcaactgacggtacagccgaaaaaggcggcgacctcggctgggtcggcaaagncgataacatggacaaggatttcgtcaaagcggcatttgctttgaaaaccggcgaaatcagcggacctgtgaaatcccaattcggctatcacatcattaaaaaagacgaagaacgcggcaaatatgaagacatgaaaaaagagcttaaaaaagaagtccaagaacaaaagcaaaatgatcaaactgaactgcaatccgtcattgacaaacttgtcaaagatgctgatttaaaagtaaaagacaaagagttgaaaaaacaagtcgaccagcgtcaagctcagacaagcagcagcagctgacgccaaaaaagctgtcctcccctcgttggggtcggacagctttttttatgcgatggaatggctgtcagctgcaggtacccgggtctagagtcgacgcggccgtatacttaatttcctttaagcctgtactttttgccatctattgatatcgtgaaatttgaaggaccgctgatcggcaaataatagacaagctgaaactccgcttcctcaccaggtttaatggttttccagcttgatagagtcactctgatgcggtggaaatctcctgtcagtccgccaatgttcggtcccgtatgccctttagaaatcacttcaacatgatcgccgctccagcttctaaaccgaggggaagtggatgtcggcgcatcaaattcgataaaagaacctcccgtaatcgtgacatcgctgttgttgatcagcttcatcaccgggtggatcgggtaattttgatctcctaacggaaagttcgtcaactctacttctgcatctagtgattcatcaggcaaaggtgatgtgctgagtcgattatcgtatggtgttgatccgagaatttatgatactcgttctcgttaaggtattgccgataccgtattctcctttactgccgtttcgttcgggataccagtcataatcag

SEQ ID NO: 7 - DNA, 10063 bp, Artificial Sequence
  Plasmid pBKQ3825 (plasmid with insertion of mad7d in between forD flanks)
gaattcgtacgagttcctccacattcggagtatttctgaatgatagagccacacggtccacgttctcactggctaaccggatcaaatgatcttcaggagtcagcataatacatccagttcaggtagataagatttgaatttggtgacttgcttttgttcttcttctttcattttctgactaatccaaactggaaaaagcaggtcttttaacagattaggaggtttctgacatgcaccattcggtcactaaccgaatgcagtaaaggacactgtggtgcttgccagccattagggtattgaggaggtgatcaaaatgctaggtgacagtatttcgtcgaagtggacaagtcgtgaccaaatgacctcggatcgagggttggtcatggaggaaaaaattgatgtctggtgacaaagaggagtcatgatcatggcaccgccaacgagggaaaaaactcttcccgcatcgacacggtatgtgggcggtgacaaactaacttatagagtaaatttattagtcgaatgaaagacgcgctaaaaatgaggagggaagcgaatgaataatggcacaaataacttccagaacttcattggcattagcagcctgcaaaaaacactgagaaatgcactgattccgacagaaacaacacagcagtttattgtcaaaaacggcatcatcaaagaggatgaactgagaggcgaaaatcgccaaattctgaaagatatcatggacgactattaccgtggctttatttcagaaacactgtccagcattgatgatatcgattggacaagcctgttcgagaaaatggaaatccaactgaaaaacggcgataacaaagacacgctgattaaagaacaaacggaatatcgcaaagcgatccacaaaaagtttgcaaatgatgaccgctttaaaaacatgttcagcgcgaaactgattagcgatattctgccggaatttgtcatccacaataataactatagcgcgagcgagaaagaagaaaaaacacaggtcattaaactgtttagccgctttgccacaagcttcaaagactatttcaaaaatcgcgcaaactgctttagcgcagatgatatttcatcatcaagctgccatcggattgtcaatgataatgcggaaatcttttttagcaacgcactggtctatcgcagaattgttaaatcattgagcaacgacgacatcaacaaaatctcaggcgatatgaaagacagcctgaaagaaatgtcactggaagaaatctacagctacgaaaaatacggcgaatttatcacacaagaaggcatcagcttttacaacgatatttgcggcaaagtcaacagctttatgaatctgtattgccagaaaaacaaagaaaacaaaaacctgtataaactgcagaaactgcacaagcagattctgtgcattgcagatacatcatatgaagtcccgtacaaatttgagagcgacgaagaagtttatcaaagcgttaatggctttctggataacatcagcagcaaacatattgttgaacgcctgagaaaaattggcgataactataatggctacaacctggacaaaatctacatcgtcagcaaattttacgaaagcgtcagccaaaaaacatatcgcgattgggaaacaattaatacagcgctggaaattcattataacaacattctgcctggcaacggcaaaagcaaagcagataaagttaaaaaggcggtcaaaaatgacctgcagaaaagcattacagaaatcaatgaactggtcagcaactacaaactgtgctcagatgataatatcaaggcggaaacgtacatccatgaaattagccatatcctgaacaactttgaagcgcaagaactgaaatataacccggaaatccatctggttgaaagcgaactgaaagcaagcgagctgaaaaatgttctggatgtcattatgaatgcgtttcattggtgcagcgtctttatgacagaagaactggtcgataaagataacaacttttatgcggaactggaagagatttacgacgaaatttatccggtcatcagcctgtataatctggttcgcaattatgtcacacagaaaccgtatagcacgaagaaaatcaaactgaactttggcattccgacactggcagatggctggtcaaaatcaaaagaatatagcaacaacgcgatcatcctgatgcgcgataatctttattatctgggcattttcaacgcgaaaaacaagccggacaaaaaaatcatcgaaggcaatacgtcagagaacaaaggcgactataaaaagatgatctataatctgcttccgggaccgaataaaatgatcccgaaagtttttctgtcaagcaaaacaggcgtcgaaacatataaaccgtcagcgtatattctggaaggctacaaacagaacaaacacatcaaaagcagcaaggactttgacatcacattttgccatgatctgatcgactactttaagaactgcattgcaattcatccggaatggaaaaacttcggctttgatttttcagacacgagcacgtatgaagatatcagcggcttttatagagaagttgaactgcagggctataaaatcgactggacatatatcagcgaaaaggatattgatctgctgcaagaaaaaggccaactgtacctgtttcagatctacaacaaagacttcagcaaaaaaagcacgggcaatgataacctgcatacgatgtacctgaaaaacctttttagcgaagagaacctgaaagacattgtcctgaaactgaatggcgaagccgaaattttctttcgcaaatccagcattaaaaacccgatcatccataaaaaaggcagcattctggttaaccgcacatatgaagcggaagaaaaagatcagtttggcaacattcagatcgtccgcaaaaacattccggaaaacatttatcaagaactgtacaaatactttaacgataaaagcgataaagaactgtccgacgaagcagcgaaacttaaaaatgttgttggccatcatgaagcggcaacaaacattgttaaagactatcgctatacgtacgataaatactttctgcatatgccgatcacgatcaacttcaaagcaaataaaacgggctttatcaacgatcgcattctgcagtatattgccaaagaaaaggatctgcatgtcatcggcattgctagaggcgaacgcaatctgatttatgtcagcgttattgatacatgcggcaacattgtcgaacagaaaagctttaacattgtcaacggctatgactaccagatcaagctgaaacagcaagaaggcgcaagacaaattgctcgcaaagaatggaaagaaatcggcaagatcaaagaaattaaagagggctatctgagcctggtcattcatgaaatttctaaaatggtcatcaaatataacgcgattatcgccatggaagatctgtcatatggctttaagaaaggccgttttaaagtcgaaagacaggtctaccagaaattcgaaacaatgctgattaacaaactgaattatctggtgtttaaagacatcagcatcacggaaaatggcggactgctgaaaggctatcaactgacatatattccggataagcttaaaaacgtcggccatcaatgcggctgcatcttttatgttccggcagcgtatacatcaaaaattgatccgacaacaggctttgtcaacatcttcaaattcaaagatctgacggtcgatgcgaaacgcgaattcattaagaaatttgacagcatccgctacgacagcgagaaaaatcttttctgctttacgttcgactacaacaactttatcacgcagaatacggttatgtcaaaaagcagctggtcagtctatacatatggcgttagaattaaacgcagatttgtgaacggcagatttagcaatgaaagcgatacaatcgacatcacgaaagacatggaaaaaacgcttgaaatgacggatattaactggcgtgatggacatgatcttcgccaggatattatcgattatgaaatcgtccagcacatctttgaaatctttagactgacagtccaaatgcgcaattcactgtcagaacttgaagatagagattatgatcgcctgatttctccggtcctgaatgaaaataacatcttttacgatagcgcaaaagcaggcgacgcactgccgaaagatgcggatgcaaatggcgcatattgcattgcactgaaaggcctgtatgaaatcaaacaaatcaccgagaattggaaagaggacggcaaattttcacgggataaactgaaaatcagcaacaaggactggtttgacttcatccaaaataagcgctacctgtaatgctgtccagactgtccgctgtgtaaaaaaaaggaataaaggggggttgacattattttactgatatgtataatataatttgtataagaaaatggtcaaaagacctttttaatttctactcttgtagatacaagtaccattttccctatagcggccgcagatctgggaccaataataatgactagagaagaaagaatgaagattgttcatgaaattaaggaacgaatattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactggctagcatggcaagacccagaaaagttctgggagatcccgctttgcataagcgtattatagtggatgacgcgggctttgttgtttacacttcttgcacctgctgacggcaatcatccctatctatgaaatcgagatttcagcaggccgttattttcgagagagttaaatctatattcattgtttttattttggtaaggacataccggattttaggtttggattaccggtcgagttagcttgtcttttcgcccactaccgtgtcgatgcgggagcaatttaccagaagcacttaccgattgatagttttttattccggtgattgcaaagtttcataaacaagcttgcatgcctgcaggtcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatggatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggtttcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatggttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgactctagaggatccccgggtaccgagctctgccttttagtccagctgatttcactttttgcattctacaaactgcataactcatatgtaaatcgctcctttttaggtggcacaaatgtgaggcattttcgctctttccggcaaccacttccaagtaaagtataacacactatactttatattcataaagtgtgtgctctgcgaggctgtcggcagtgccgaccaaaaccataaaacctttaagacctttcttttttttacgagaaaaaagaaacaaaaaaacctgccctctgccacctcagcaaaggggggttttgctctcgtgctcgtttaaaaatcagcaagggacaggtagtattttttgagaagatcactcaaaaaatctccacctttaaacccttgccaatttttattttgtccgttttgtctagcttaccgaaagccagactcagcaagaataaaatttttattgtctttcggttttctagtgtaacggacaaaaccactcaaaataaaaaagatacaagagaggtctctcgtatcttttattcagcaatcgcgcccgattgctgaacagattaataatgagctcg

SEQ ID NO: 8 - DNA, 5722 bp, Artificial Sequence
  forDmad7d-cat, Fig. 5 (BKQ3934)
cgtacgagttcctccacattcggagtatttctgaatgatagagccacacggtccacgttctcactggctaaccggatcaaatgatcttcaggagtcagcataatacatccagttcaggtagataagatttgaatttggtgacttgcttttgttcttcttctttcattttctgactaatccaaactggaaaaagcaggtcttttaacagattaggaggtttctgacatgcaccattcggtcactaaccgaatgcagtaaaggacactgtggtgcttgccagccattagggtattgaggaggtgatcaaaatgctaggtgacagtatttcgtcgaagtggacaagtcgtgaccaaatgacctcggatcgagggttggtcatggaggaaaaaattgatgtctggtgacaaagaggagtcatgatcatggcaccgccaacgagggaaaaaactcttcccgcatcgacacggtatgtgggcggtgacaaactaacttatagagtaaatttattagtcgaatgaaagacgcgctaaaaatgaggagggaagcgaatgaataatggcacaaataacttccagaacttcattggcattagcagcctgcaaaaaacactgagaaatgcactgattccgacagaaacaacacagcagtttattgtcaaaaacggcatcatcaaagaggatgaactgagaggcgaaaatcgccaaattctgaaagatatcatggacgactattaccgtggctttatttcagaaacactgtccagcattgatgatatcgattggacaagcctgttcgagaaaatggaaatccaactgaaaaacggcgataacaaagacacgctgattaaagaacaaacggaatatcgcaaagcgatccacaaaaagtttgcaaatgatgaccgctttaaaaacatgttcagcgcgaaactgattagcgatattctgccggaatttgtcatccacaataataactatagcgcgagcgagaaagaagaaaaaacacaggtcattaaactgtttagccgctttgccacaagcttcaaagactatttcaaaaatcgcgcaaactgctttagcgcagatgatatttcatcatcaagctgccatcggattgtcaatgataatgcggaaatcttttttagcaacgcactggtctatcgcagaattgttaaatcattgagcaacgacgacatcaacaaaatctcaggcgatatgaaagacagcctgaaagaaatgtcactggaagaaatctacagctacgaaaaatacggcgaatttatcacacaagaaggcatcagcttttacaacgatatttgcggcaaagtcaacagctttatgaatctgtattgccagaaaaacaaagaaaacaaaaacctgtataaactgcagaaactgcacaagcagattctgtgcattgcagatacatcatatgaagtcccgtacaaatttgagagcgacgaagaagtttatcaaagcgttaatggctttctggataacatcagcagcaaacatattgttgaacgcctgagaaaaattggcgataactataatggctacaacctggacaaaatctacatcgtcagcaaattttacgaaagcgtcagccaaaaaacatatcgcgattgggaaacaattaatacagcgctggaaattcattataacaacattctgcctggcaacggcaaaagcaaagcagataaagttaaaaaggcggtcaaaaatgacctgcagaaaagcattacagaaatcaatgaactggtcagcaactacaaactgtgctcagatgataatatcaaggcggaaacgtacatccatgaaattagccatatcctgaacaactttgaagcgcaagaactgaaatataacccggaaatccatctggttgaaagcgaactgaaagcaagcgagctgaaaaatgttctggatgtcattatgaatgcgtttcattggtgcagcgtctttatgacagaagaactggtcgataaagataacaacttttatgcggaactggaagagatttacgacgaaatttatccggtcatcagcctgtataatctggttcgcaattatgtcacacagaaaccgtatagcacgaagaaaatcaaactgaactttggcattccgacactggcagatggctggtcaaaatcaaaagaatatagcaacaacgcgatcatcctgatgcgcgataatctttattatctgggcattttcaacgcgaaaaacaagccggacaaaaaaatcatcgaaggcaatacgtcagagaacaaaggcgactataaaaagatgatctataatctgcttccgggaccgaataaaatgatcccgaaagtttttctgtcaagcaaaacaggcgtcgaaacatataaaccgtcagcgtatattctggaaggctacaaacagaacaaacacatcaaaagcagcaaggactttgacatcacattttgccatgatctgatcgactactttaagaactgcattgcaattcatccggaatggaaaaacttcggctttgatttttcagacacgagcacgtatgaagatatcagcggcttttatagagaagttgaactgcagggctataaaatcgactggacatatatcagcgaaaaggatattgatctgctgcaagaaaaaggccaactgtacctgtttcagatctacaacaaagacttcagcaaaaaaagcacgggcaatgataacctgcatacgatgtacctgaaaaacctttttagcgaagagaacctgaaagacattgtcctgaaactgaatggcgaagccgaaattttctttcgcaaatccagcattaaaaacccgatcatccataaaaaaggcagcattctggttaaccgcacatatgaagcggaagaaaaagatcagtttggcaacattcagatcgtccgcaaaaacattccggaaaacatttatcaagaactgtacaaatactttaacgataaaagcgataaagaactgtccgacgaagcagcgaaacttaaaaatgttgttggccatcatgaagcggcaacaaacattgttaaagactatcgctatacgtacgataaatactttctgcatatgccgatcacgatcaacttcaaagcaaataaaacgggctttatcaacgatcgcattctgcagtatattgccaaagaaaaggatctgcatgtcatcggcattgctagaggcgaacgcaatctgatttatgtcagcgttattgatacatgcggcaacattgtcgaacagaaaagctttaacattgtcaacggctatgactaccagatcaagctgaaacagcaagaaggcgcaagacaaattgctcgcaaagaatggaaagaaatcggcaagatcaaagaaattaaagagggctatctgagcctggtcattcatgaaatttctaaaatggtcatcaaatataacgcgattatcgccatggaagatctgtcatatggctttaagaaaggccgttttaaagtcgaaagacaggtctaccagaaattcgaaacaatgctgattaacaaactgaattatctggtgtttaaagacatcagcatcacggaaaatggcggactgctgaaaggctatcaactgacatatattccggataagcttaaaaacgtcggccatcaatgcggctgcatcttttatgttccggcagcgtatacatcaaaaattgatccgacaacaggctttgtcaacatcttcaaattcaaagatctgacggtcgatgcgaaacgcgaattcattaagaaatttgacagcatccgctacgacagcgagaaaaatcttttctgctttacgttcgactacaacaactttatcacgcagaatacggttatgtcaaaaagcagctggtcagtctatacatatggcgttagaattaaacgcagatttgtgaacggcagatttagcaatgaaagcgatacaatcgacatcacgaaagacatggaaaaaacgcttgaaatgacggatattaactggcgtgatggacatgatcttcgccaggatattatcgattatgaaatcgtccagcacatctttgaaatctttagactgacagtccaaatgcgcaattcactgtcagaacttgaagatagagattatgatcgcctgatttctccggtcctgaatgaaaataacatcttttacgatagcgcaaaagcaggcgacgcactgccgaaagatgcggatgcaaatggcgcatattgcattgcactgaaaggcctgtatgaaatcaaacaaatcaccgagaattggaaagaggacggcaaattttcacgggataaactgaaaatcagcaacaaggactggtttgacttcatccaaaataagcgctacctgtaatgctgtccagactgtccgctgtgtaaaaaaaaggaataaaggggggttgacattattttactgatatgtataatataatttgtataagaaaatggtcaaaagacctttttaatttctactcttgtagatacaagtaccattttccctatagcggccgcagatctgggaccaataataatgactagagaagaaagaatgaagattgttcatgaaattaaggaacgaatattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactggctagcatggcaagacccagaaaagttctgggagatcccgctttgcataagcgtattatagtggatgacgcgggctttgttgtttacacttcttgcacctgctgacggcaatcatccctatctatgaaatcgagatttcagcaggccgttattttcgagagagttaaatctatattcattgtttttattttggtaaggacataccggattttaggtttggattaccggtcgagttagcttgtcttttcgcccactaccgtgtcgatgcgggagcaatttaccagaagcacttaccgattgatagttttttattccggtgattgcaaagtttcataaacaagct

SEQ ID NO: 9 - DNA, 9200 bp, Artificial Sequence
  amyEGFP-gDNA(gfp)-spc, Fig 8 (PP.5625)
aagcagtacatgctgggcatgtactgcttttttctattacactcctaatcttgcaagagtaaacgtccatatacaaatgatcaataacagaatcaggctgtacggcactgtccttttaaacagtttagattccttgcctgtttggccgaccgctgcggcagcgatggcgatagattgaggagaaacaagtttcgccatgacaccgcccgccgtatttgcggctaacagcaaatctgaacccgcaccgatctgcgctcccgtgacaacctgcaaatggccgaacaaagcattattgctgacaacagaaccggtgatgaacacgccaatccagccgagaacaggactgacaaacgggaacaggtctcctgttctcgccaatgctaacccaattgaagagctgagccctgcgaagttggccagattggcaaaccccatcacgaagcagatcgtcataatcggcacccacagctcttttcccgtctccttcaaggaagcaaaggcatcccgagaggagaactttttgctgaccagccctgtcacgatgaccgccgctaaaatcgctgtaccagtcgctgacagcagatcaactttaaagactgcatctaacggcatggcagatggcgcaatcggcggcattttcataatttgctgatgcagaaaaggcattttgaacaggagcgttgattgatataacagcccgccttcttggaaaagcgctttgaaggcaggaaggctccagatggtgatcgccgcagttaaaatgtagaaaggagaccacgctctcgcaatgtcagcggcacggtatgcctttttctcaccagcatcgccggccccttcctcgcggtaaatctctttcggctgccatttgcggaggaagaaggcaagcccgcccatgctgaataaggccgccaaaatgtttgctaattccggcccgagcaccgccattgtcagtgtctgaaccgctgtatacccaccgctcacaaccagaagagcgggccatgtctgtttgattcctttcattcggtctaataagaatacaagcaggaatggtattaaaaatgagatcatcggcagtgtccacattaatgtccgagacagctcaagagcagacaaatcaccaatctgcgcccctgtgatgacaggaatcccaatcgccccaaaggctccggaggcagcgtttgcaatcaagcagagcgccgccgcttttaacggtttaaaaccaagttcgacgagcagcgccgcactaatcgcaatcggaacaccaaaaccggccgcgccttctaaaaacgcgttaaaacaaaaaccaattaataatagctgtaatcgttggtcaggcgaaatgccggcaatgctggaccgaataatggtaaatttcccggttttcacggcgattttatacagccacaccgccatcaggacgatgtagccaatgggccacagcccgctcccgattcctaacaaaacagaagaaatcgctttttcaaccggcatatgaaatgcccaaacggagacgaagaaactgacggctagggtaagaaatgccgcaagaatgcctttcattttcaaaacagttaaagccaaaagaaaaaagagaatcggagtgagcgccacaagtgcgctcacatactcgtttccaaacggatcatacaactgctcccacattgacaatcagccctttactctaaagttgcggttagttgactttttgttctgcaaaatgaggttttaaaatgtttttaaggacgccggcgctgtgaaggaactgttctttttctttctcatttaagttcagctcagtgatacctgcgatccctccgcgattcacgacagccggcacaccgatgtacacgtcatctgcaccgtattgcccgtccaaatatgtgctgacagttaatatgctgttttcattatgaagaatggcttttgtaatgcgagcaagactcatcgcaaccccataataagtcgcgccttttttctcaatgatatggtaagctgcgtttttcacatcatctacaatttggtccagctcctcttgtttgtacgcatcgtttttctcaacgagttcactgaccggcacaccgccgacattcgcgtggctccaaacaggaagctctgtgtcgccgtgctctccgataatatgcgcgtgtacgttttgaggcgctgcgccaaagtattcgctcagcatgaaacggaatctcgcagaatcaagtgttgtgccgcttccaatcacccgctcttttggcaggccgctgaatttccatgttgcgtaagtcaggatatcaaccggatttgtcgcgactaagaaaatgccgtcaaatccgctcgccatgacttcactaacgatgcctttgaaaatcttcaagttcttttctactaattcaaggcgtgtctcaccaggtttttggtttgctccggcgcaaatgcagacaatatcagcatccttgcagtcttcatatgttccgtaagatgttttgaccggttgtagcccaaacgcctttccgtggggtaaatccatcacatcgcccattgctttttctttatttacatcaatgaccacaagctcatctgtgatcccttggttaattaacgcaaatgcataactgcttccaacaaaacccgctccgattaaagctactttatttacatgtttgttcatcattaatcatccttgcagggtatgtttctctttgatgtctttttgtttgtgaagtatttcacatttatattgtgcaacacttcacaaacttttgcaagagaaaagttttgtctgatttatgaacaaaaaagaaaccatcattgatggtttctttcggtaagtcccgtctagccttgccctcaatggggaagagaaccgcttaagcccgagtcattatataaaccatttagcacgtaatcaaagccaggctgattctgaccgggcacttgggcgctgccattattaaaaatcacttttgcgttggttgtatccgtgtccgcaggcagcgtcagcgtgtaaattccgtctgcatttttagtcattggttttccaggccaagatccggtcaattcaattactcggctcccatcatgtttatagatataagcatttacctggctccaatgattcggattttgatagccgatggttttggccgacgctggatctcttttaacaaaactgtatttctcggtcctcgttacaccatcactgttcgttccttttaacatgatggtgtatgttttgccaaattggatctccttttccgattgtgaattgatctccatccttaaacgcctgtcgtctggtccattattgatttgataaacggcttttgttgtattcgcatctgcacgcaaggtaatcgtcagttgatcattgaaagaatgtgttacacctgttttgtaattctcaaggaaaacatgaggcgcttttgcaatatcatcaggataaagcacagctacagacctggcattgatcgtgcctgtcagtttaccatcgttcacttgaaatgaacccgctccagctttattgtcatacctgccatcaggcaattttgttgccgtattgatagagacagaggatgaacctgcatttgccagcacaacgccatgtgagccgcgctgattcataaatatctggttgtttccattcgggttcgagagttcctcaggctgtccagccatcacattgtgaaatctattgaccgcagtgatagcctgatcttcaaataaagcactcccgcgatcgcctatttggcttttccccgggaacctcacaccatttccgcctccctcaggtctggaaaagaaaagaggcgtactgcctgaacgagaagctatcaccgcccagcctaaacggatatcatcatcgctcatccatgtctgtagacaaattgtgaaaggatgtacttaaacgctaacggtcagctttattgaacagtaatttaagtatatgtccaatctagggtaagtaaattgagtatcaatataaactttatatgaacataatcaacgaggtgaaatcatgagcaatttgattaacggaaaaataccaaatcaagcgattcaaacattaaaaatcgtaaaagatttatttggaagttcaatagttggagtatatctatttggttcagcagtaaatggtggtttacgcatttacagcgatgtagatgttctagtcgtcgtgaatcatagtttacctcaattaactcgaaaaaaactaacagaaagactaatgactatatcaggaaagattggaaatacggattctgttagaccacttgaagttacggttataaataggagtgaagttgtcccttggcaatatcctccaaaaagagaatttatatacggtgagtggctcaggtgtggatttgagaatggacaaattcaggaaccaagctatgatcctgatttggctattgttttagcacaagcaagaaagaatagtatttctctatttggtcctgattcttcaagtatacttgtctccgtacctttgacagatattcgaagagcaattaaggattctttgccagaactaattgaggggataaaaggtgatgagcgtaatgtaattttaaccctagctcgaatgtggcaaacagtgactactggtgaaattacctcgaaagatgtcgctgcggaatgggctatacctcttttacctaaagagcatgtaactttactggatatagccagaaaaggctatcggggagagtgtgatgataagtgggaaggactatattcaaaggtgaaagcactcgttaagtatatgaaaaattctatagaaacttctctcaattaggctaattttattgcaataacaggtgcttacttttaaaactactgatttattgataaatattgaacaatttttgggaagaataaagcgtcctcttgtgaaattagagaacgctttattactttaatttagtgaaacaatttgtaactattgaaaatagaaagaaattgttccttcgatagtttattaatattagtggagctcagtgagagcgaagcgaacacttgattttttaattttctatcttttataggtcattagagtatacttatttgtcctataaactatttagcagcataatagatttattgaataggtcatttaagttgagcatattaggggaggaaaatcttggagaaatatttgaagaacccgagatctagatcaggtaccgcaacgttcgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtacttgttaaaaattcggaatatttatacaatatcatatgtatcacattgaaaggaggagggaagctttatgagtaaaggagaagaacttttcactggagttgtcccaattcttgttgaattagatggcgatgttaatgggcaaaaattctctgttagtggagagggtgaaggtgatgcaacatacggaaaacttacccttaaatttatttgcactactgggaagctacctgttccatggccaacgcttgtcactactctcacttatggtgttcaatgcttttctagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaattataactcacataatgtatacatcatggcagacaaaccaaagaatggcatcaaagttaacttcaaaattagacacaacattaaagatggaagcgttcaattagcagaccattatcaacaaaatactccaattggcgatggccctgtccttttaccagacaaccattacctgtccacgcaatctgccctttccaaagatcccaacgaaaagagagatcacatgatccttcttgagtttgtaacagctgctgggattacacatggcatggatgaactatacaaataatgctgtccagactgtccgctgtgtaaaaaaaaggaataaaggggggttgacattattttactgatatgtataatataatttgtataagaaaatggtcaaaagacctttttaatttctactcttgtagatactggagttgtcccaattcttattgacactaaagggatccagaagcggcaacacgctaatcaataaaaaaacgctgtgcggttaaagggcacagcgttttttgtgtatgaatcgaaaaagagaacagatcgcaggtctcaaaaatcgagcgtaaagggctgatccgcggccgcgcgtcaacaatgacctttatgccatattcttcagcggctgcacacatttctttaaattcttgttcagtacctaagtaacggttgccaatttgatacgatgtcggctgatacagccagtaccagttcgacatgcttttatctccttgattcccttcctttacttggttaatcggagatgtctgaatggctgtatatcctgcatcatgaatatccttcatattgtgttttaacgtattgaacgaccaattccatgcatgaagaatggttccgcttttgatcgacggtgctgtaagctcattcgatttgttcgccgtttcagcactcgcagccgccggtcctgccagaaccaaatgaaacagcaataaaaatccagcgaataacggcagtaaagaggttttgaatcgttttgcaaacattcttgacactccttatttgattttttgaagacttacttcggagtcaaaaatccctcttacttcattcttccgcttcctcctttcaaaccgatgtgaagactggagaattttgttaacgcttacatttaaaattatcacaatcactctatcaaaacaacttggcagagtgaatacaaatcaatgttcctataaaaaaagctgcccgaaactgtgaacgcttccctcgctttccaaacaaaaaagatgatttcttttttatttcttcttttaccgctcttctgtaagccagcttcacaatcacataccgaagcagaaacagaacgccgccgcagacgataaaaagaagaatcattttcagccaagcgaagaatgtatggggaatccccctgtcgtacatcgtaagcaccactaccgcaaaggcgcaagcaattgattctattcgctgctgcttcacccgtttttgcagttcttctttggtgtctattctcggcatgagcattccggatgccgcagccctgatgcagtaatataagataatgatgaacaaaaacacaaggcttgcagaccattctttaaacggaaattcaaacataatgccgcggatgatcacatcaggcagagcaactgctcccgctatcatcccattctccgaccagattttttgttctttttcttttactctcttatttttcccattcacgcctgaaagtctattacgcagattattcatgaccttcctcccgcacttttacgaatggcttatggcttacatgttacaataacagcctgcgcttctgcaatgaataactcaaaagcccgccgtacaagccggcgggcagcaatcgttaaaacggcccccaattgatcatgactccgatcacgataaagacaagcccgatcaagaaccaaatcagagcaagcggcaccataaaccgcagccacttgacatacggaataccgctggcggcaagcaccgccatgagaacgccggatgtcgggttcacacagttgacgaccccttctccaagcataaccgcttcaaccgcaacctgtctcgtgattcccatcaaatcagcgagcggcgccaggattggaataaatacaacggcttcgccagaacctgaagagatgagaaaatgaagcagcgcactggcgatatacatgccgattgccccagcaatcgggctgaatccatccaaaagtgaagccaaagcattgacgacagtatcgagaagctttccattttcaaggataacggaaatgcttcgtgccatcccgacaatcagcgccccgtatacaagactttggcagcccgtaatgaaggttttggcaatatcgttcgccgcaagcccgcctattaaaccggcaaggacagaaataaaaataaatgtcgcagccatttgggaatctgaccagccaagcttcaaagcgccgtataaaaatccgacaagtgagagtcccgccacagccaaaatcagcttatggcgaacggtaaacggcactgactgatcttcttctttaccggcttcgcccattcctgccgcagggaaccaatctgttcctaacacactggcatcttttgatttttttaattttctcgtatacaaatagatataaataatagaagacagcaaaaagcatatgtatatcacaactcgcaggccgattcctgagaaaagagggagctccgcgattgattgtgataaaccgagcggtgacggtgataatatggtggagttaaatccagcgtagcagccgatgtatataacagcggctcccgcgactgcgtcccattttaaggatcgtgccacaatgaggccgatggggatacaaccgataactgaattcacgacaatcccggttgttccgagaatggagaacaatgcgccgacaatacaaatacataataattgctttgtgcggaatttgctgatgacattgtaaatcaggccattgatggcacccgttttttctagaatagcaatggtgccgcctgtgaataaaatcagaaagatgatgggtgctgatccaaccattccatcctgtagagaagtaaaaaagctgatcaatctgaccggagactgttcaattgaatgatagcttcccggaacagcggtcgtgacatccccctttgtcacgcggtcaaattcacctgccggcacaatatatgaagcgattgcgcaaatgaaagcaatcataaataagaggacataggcgtcaggcattttaaatgtcatacgttttttcttaggctgtgtttcggctgctggcatactcatatttcgatccatcccctctgtttgaaaatattaattattctgaatagtagcaatatgtgctatattaa

SEQ ID NO: 10 - DNA, 8704 bp, Artificial Sequence
  pelmad7d, Fig. 9 (MOL3268)
ttactgctttctcatgaaagcatcatcagacacaaataagtggtatgcagcgttaccgtgtcttcgagacaaaaacgcatgggcgttggctttagaggtttcgaacatatcagcagtgacataaggaaggagagtgctgagataaccggacaatttcttttctatttcatctgttagtgcaaattcaatgtcgccgatattcatgataatcgagaaaacaaagtcgatatcgatatgaaaatgttcctcggcaaaaaccgcaagctcgtgaattcctggtgaacatccggcacgcttatggaaaatctgtttgactaaatcactcacaatccaagcattgtattgctgttctggtgaaaagtattgcattagacatacctcctgctcgtacggataaaggcagcgtttcatggtcgtgtgctccgtgcagcggcttctccttaattttgatttttctgaaaataggtcccgttcctatcactttaccatggacggaaaacaaatagctactaccattcctcctgtttttctcttcaatgttctggaatctgtttcaggtacagacgatcgggtatgaaagaaatatagaaaacatgaaggaggaatatcgacatgaaaccagttgtaaaagagtatacaaatgacgaacagctcatgaaagatgtagaggaattgcagaaaatgggtgttgcgaaagaggatgtatacgtcttagctcacgacgatgacagaacggaacgcctggctgacaacacgaacgccaacacgatcggagccaaagaaacaggtttcaagcacgcggtgggaaatatcttcaataaaaaaggagacgagctccgcaataaaattcacgaaatcggtttttctgaagatgaagccgctcaatttgaaaaacgcttagatgaaggaaaagtgcttctctttgtgacagataacgaaaaagtgaaagcttgggcataaagcaaggaaaaaaccaaaaggccaatgtcggccttttggtttttttgcggtctttgcggtgggattttgcagaatgccgcaataggatagcggaacattttcggttctgaatgtccctcaatttgctattatatttttgtgataaattggaataaaatctcacaaaatagaaaatgggggtacatagtggatgaaaaaagtgatgttagctacggctttgtttttaggattgactccagctggcgcgaacgcagctgatttaggccaccagacgttgggatccaatgatggctggggcgcgtactcgaccggcacgacaggcggatcaaaagcatcctcctcaaatgtgtataccgtcagcaacagaaaccagcttgtctcggcattagggaaggaaacgaacacaacgccaaaaatcatttatatcaagggaacgattgacatgaacgtggatgacaatctgaagccgcttggcctaaatgactataaagatccggagtatgatttggacaaatatttgaaagcctatgatcctagcacatggggcaaaaaagagccgtcgggaacacaagaagaagcgagagcacgctctcagaaaaaccaaaaagcacgggtcatggtggatatccctgcaaacacgacgatcgtcggttcagggactaacgctaaagtcgtgggaggaaacttccaaatcaagagtgataacgtcattattcgcaacattgaattccaggatgcctatgactattttccgcaatggttgtaaaacgacggccagtgaattctgatcaaatggttcagtgagagcgaagcgaacacttgattttttaattttctatcttttataggtcattagagtatacttatttgtcctataaactatttagcagcataatagatttattgaataggtcatttaagttgagcatattagaggaggaaaatcttggagaaatatttgaagaacccgaggatctagatcaggtaccgcaacgttcgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtacttgttaaaaattcgaaatatttatacaatatcatatgtatcacattgaaaggggaggagaatcatgaataatggcacaaataacttccagaacttcattggcattagcagcctgcaaaaaacactgagaaatgcactgattccgacagaaacaacacagcagtttattgtcaaaaacggcatcatcaaagaggatgaactgagaggcgaaaatcgccaaattctgaaagatatcatggacgactattaccgtggctttatttcagaaacactgtccagcattgatgatatcgattggacaagcctgttcgagaaaatggaaatccaactgaaaaacggcgataacaaagacacgctgattaaagaacaaacggaatatcgcaaagcgatccacaaaaagtttgcaaatgatgaccgctttaaaaacatgttcagcgcgaaactgattagcgatattctgccggaatttgtcatccacaataataactatagcgcgagcgagaaagaagaaaaaacacaggtcattaaactgtttagccgctttgccacaagcttcaaagactatttcaaaaatcgcgcaaactgctttagcgcagatgatatttcatcatcaagctgccatcggattgtcaatgataatgcggaaatcttttttagcaacgcactggtctatcgcagaattgttaaatcattgagcaacgacgacatcaacaaaatctcaggcgatatgaaagacagcctgaaagaaatgtcactggaagaaatctacagctacgaaaaatacggcgaatttatcacacaagaaggcatcagcttttacaacgatatttgcggcaaagtcaacagctttatgaatctgtattgccagaaaaacaaagaaaacaaaaacctgtataaactgcagaaactgcacaagcagattctgtgcattgcagatacatcatatgaagtcccgtacaaatttgagagcgacgaagaagtttatcaaagcgttaatggctttctggataacatcagcagcaaacatattgttgaacgcctgagaaaaattggcgataactataatggctacaacctggacaaaatctacatcgtcagcaaattttacgaaagcgtcagccaaaaaacatatcgcgattgggaaacaattaatacagcgctggaaattcattataacaacattctgcctggcaacggcaaaagcaaagcagataaagttaaaaaggcggtcaaaaatgacctgcagaaaagcattacagaaatcaatgaactggtcagcaactacaaactgtgctcagatgataatatcaaggcggaaacgtacatccatgaaattagccatatcctgaacaactttgaagcgcaagaactgaaatataacccggaaatccatctggttgaaagcgaactgaaagcaagcgagctgaaaaatgttctggatgtcattatgaatgcgtttcattggtgcagcgtctttatgacagaagaactggtcgataaagataacaacttttatgcggaactggaagagatttacgacgaaatttatccggtcatcagcctgtataatctggttcgcaattatgtcacacagaaaccgtatagcacgaagaaaatcaaactgaactttggcattccgacactggcagatggctggtcaaaatcaaaagaatatagcaacaacgcgatcatcctgatgcgcgataatctttattatctgggcattttcaacgcgaaaaacaagccggacaaaaaaatcatcgaaggcaatacgtcagagaacaaaggcgactataaaaagatgatctataatctgcttccgggaccgaataaaatgatcccgaaagtttttctgtcaagcaaaacaggcgtcgaaacatataaaccgtcagcgtatattctggaaggctacaaacagaacaaacacatcaaaagcagcaaggactttgacatcacattttgccatgatctgatcgactactttaagaactgcattgcaattcatccggaatggaaaaacttcggctttgatttttcagacacgagcacgtatgaagatatcagcggcttttatagagaagttgaactgcagggctataaaatcgactggacatatatcagcgaaaaggatattgatctgctgcaagaaaaaggccaactgtacctgtttcagatctacaacaaagacttcagcaaaaaaagcacgggcaatgataacctgcatacgatgtacctgaaaaacctttttagcgaagagaacctgaaagacattgtcctgaaactgaatggcgaagccgaaattttctttcgcaaatccagcattaaaaacccgatcatccataaaaaaggcagcattctggttaaccgcacatatgaagcggaagaaaaagatcagtttggcaacattcagatcgtccgcaaaaacattccggaaaacatttatcaagaactgtacaaatactttaacgataaaagcgataaagaactgtccgacgaagcagcgaaacttaaaaatgttgttggccatcatgaagcggcaacaaacattgttaaagactatcgctatacgtacgataaatactttctgcatatgccgatcacgatcaacttcaaagcaaataaaacgggctttatcaacgatcgcattctgcagtatattgccaaagaaaaggatctgcatgtcatcggcattgctagaggcgaacgcaatctgatttatgtcagcgttattgatacatgcggcaacattgtcgaacagaaaagctttaacattgtcaacggctatgactaccagatcaagctgaaacagcaagaaggcgcaagacaaattgctcgcaaagaatggaaagaaatcggcaagatcaaagaaattaaagagggctatctgagcctggtcattcatgaaatttctaaaatggtcatcaaatataacgcgattatcgccatggaagatctgtcatatggctttaagaaaggccgttttaaagtcgaaagacaggtctaccagaaattcgaaacaatgctgattaacaaactgaattatctggtgtttaaagacatcagcatcacggaaaatggcggactgctgaaaggctatcaactgacatatattccggataagcttaaaaacgtcggccatcaatgcggctgcatcttttatgttccggcagcgtatacatcaaaaattgatccgacaacaggctttgtcaacatcttcaaattcaaagatctgacggtcgatgcgaaacgcgaattcattaagaaatttgacagcatccgctacgacagcgagaaaaatcttttctgctttacgttcgactacaacaactttatcacgcagaatacggttatgtcaaaaagcagctggtcagtctatacatatggcgttagaattaaacgcagatttgtgaacggcagatttagcaatgaaagcgatacaatcgacatcacgaaagacatggaaaaaacgcttgaaatgacggatattaactggcgtgatggacatgatcttcgccaggatattatcgattatgaaatcgtccagcacatctttgaaatctttagactgacagtccaaatgcgcaattcactgtcagaacttgaagatagagattatgatcgcctgatttctccggtcctgaatgaaaataacatcttttacgatagcgcaaaagcaggcgacgcactgccgaaagatgcggatgcaaatggcgcatattgcattgcactgaaaggcctgtatgaaatcaaacaaatcaccgagaattggaaagaggacggcaaattttcacgggataaactgaaaatcagcaacaaggactggtttgacttcatccaaaataagcgctacctgtaaacgcgttaatcgcatgttcaatccgctccataatcggtcgacgcggcggttcgcgtccggacagcacatcaccgaaatatttcgacggcccagccggctagcgcgttaatccgcggatatatagcggccgcagatctgggaccaataataatgactagagaaaaagaatgaagattgttcatgaaattaaggaacgaatattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaacgatacattaataggtacgaaaaagcaactttttttgcgcttaaaaccagtcataccaataacttaagggtaactagcctcgccggaaagagcgaaaatgcctcacatttgtgccacctaaaaaggagcgatttacatatgagttatgcagtttgtagaatgcaaaaagtgaaatcagctggactaaaaggggccgcagagtagaatggaaaaggggatcggaaaacaagtatataggaggagacctatttatggcttcagaaaaagacgcaggaaaacagtcagcagtaaagcttgttccattgcttattactgtcgctgtgggactaatcatctggtttattcccgctccgtccggacttgaacctaaagcttggcatttgtttgcgatttttgtcgcaacaattatcggctttatctccaagcccttgccaatgggtgcaattgcaatttttgcattggcggttactgcactaactggaacactatcaattgaggatacattaagcggattcgggaataagaccatttggcttatcgttatcgcattctttatttcccggggatttatcaaaaccggtctcggtgcgagaatttcgtatgtattcgttcagaaattcggaaaaaaaacccttggactttcttattcactgctattcagtgatttaatactttcacctgctattccaagtaatacggcgcgtgcaggaggcattatatttcctattatcagatcattatccgaaacattcggatcaagcccggcaaatggaacagagagaaaaatcggtgcattcttattaaaaaccggttttcaggggaatctgatcacatctgctatgttcctgacagcgatggcggcgaacccgctgattgccaagctggcccatgatgtcgcaggggtggacttaacatggacaagctgggcaattgccgcgattgtaccgggacttgtaagcttaatcatcacgccgcttgtgatttacaaactgtatccgccggaaatcaaagaaacaccggatgcggcgaaaatcgcaacagaaaaactgaaagaaatgggaccgttcaaaaaatcggagctttccatggttatcgtgtttcttttggtgcttgtgctgtggatttttggcggcagcttcaacatcgacgctaccacaaccgcattgatcggtttggccgttctcttattatcacaagttctgacttgggatgatatcaagaaagaacagggcgcttgggatacgctcacttggtttgcggcgcttgtcatgctcgccaacttcttgaatgaattaggcatggtgtcttggttcagtaatgccatgaaatcatccgtatcagggttctcttggattgtggcattcatcattttaattgttgtgtattattactctcactatttctttgcaagtgcgacagcccacatcagtgcgatgtattcagcatttttggctgtcgtcgtggcagcgggcgcaccgccgcttttagcagcgctgagcctcgcgttcatcagcaacctgttcgggtcaacgactcactacggttctggagcggctccggtcttcttcggagcaggctacatcccgcaaggcaaatggtggtccatcggatttatcctgtcgattgttcatatcatcgtatggcttgtgatcggcggattatggtggaaagtactaggaatatggtaga

SEQ ID NO: 11 - DNA, 56 bp, Artificial Sequence
  gDNA(gfp)
gtcaaaagacctttttaatttctactcttgtagatactggagttgtcccaattctt

SEQ ID NO: 12 - DNA, 56 bp, Artificial Sequence
  gDNA(P4199)
gtcaaaagacctttttaatttctactcttgtagatacaagtaccattttccctata

SEQ ID NO: 13 - DNA, 84 bp, Artificial Sequence
  Excerpt of target DNA sequence (GFP) on Fig. 14A
ggaggagggaagctttatgagtaaaggagaagaacttttcactggagttgtcccaattcttgttgaattagatggcgatgttaa

SEQ ID NO: 14 - RNA, 40 nt, Artificial Sequence
  gRNA(GFP) on Fig. 14A
aagaauugggacaacuccaguuagaugucuucaucuuuaa

SEQ ID NO: 15 - DNA, 89 bp, Artificial Sequence
  Excerpt of target DNA sequence (P4199) on Fig. 14B
ctcctttcaatgtgatacatatgatattgtataaatattccgaatttttaacaagtaccattttccctatattttcttccaaaagaaaa

SEQ ID NO: 16 - RNA, 40 nt, Artificial Sequence
  gRNA(P4199) on Fig. 14B
uauagggaaaaugguacuuguuagaugucuucaucuuuaa

SEQ ID NO: 17 - protein, 192 aa, Artificial Sequence
  Excerpt of LbCpf1 amino acid sequence on Fig. 18
IYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIEN

SEQ ID NO: 18 - protein, 189 aa, Artificial Sequence
  Excerpt of FnCpf1 amino acid sequence on Fig. 18
IYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINN

SEQ ID NO: 19 - protein, 229 aa, Artificial Sequence
  Excerpt of Mad7 amino acid sequence shown on Fig. 18
IYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGK